(19) United States
(12) Patent Application Publication
(10) Pub. No.: US 2018/0238914 A1
(43) Pub. Date: Aug. 23, 2018

(54) MEANS AND METHODS FOR DIAGNOSING CARDIAC DISEASE IN A SUBJECT

(71) Applicants: METANOMICS GMBH, Berlin (DE); RUPRECHT-KARLS-UNIVERSITÄT HEIDELBERG, Heidelberg (DE)

(72) Inventors: Philipp Schatz, Berlin (DE); Henning Witt, Berlin (DE); Erik Peter, Postdam (DE); Martin Dostler, Henningsdorf (DE); Susan Carvalho, Ludwigshafen (DE); Philipp Ternes, Berlin (DE); Philipp Mappes, Berlin (DE); Jenny Fischer, Berlin (DE); Hugo A. Katus, Heidelberg (DE); Tanja Weis, Wiesenbach (DE); Norbert Frey, Kronshagen (DE)

(73) Assignees: METANOMICS GMBH, Berlin (DE); RUPRECHT-KARLS-UNIVERSITÄT HEIDELBERG, Heidelberg (DE)

(21) Appl. No.: 15/753,388

(22) PCT Filed: Aug. 19, 2016
(86) PCT No.: PCT/EP2016/069733

§ 371 Date: Feb. 19, 2018

Related U.S. Application Data
(60) Provisional application No. 62/206,944, filed on Aug. 19, 2015.

(51) Int. Cl. G01N 33/92; G01N 33/68

(52) U.S. Cl. CPC G01N 33/92; G01N 33/6893; G01N 2405/02; G01N 2560/00; G01N 2800/325; G01N 2405/08; G01N 2405/04

(57) ABSTRACT

The present invention relates to the field of diagnostic methods. Specifically, the present invention contemplates a method for diagnosing a cardiac disease in a subject based on determining the amounts of at least three lipid metabolite biomarkers and at least one further cardiac biomarker. The invention also relates to tools for carrying out the aforementioned methods, such as diagnostic devices.

The present invention relates to the field of diagnostic methods. Specifically, the present invention contemplates a method for diagnosing a cardiac disease in a subject based on determining the amounts of at least three lipid metabolite biomarkers and at least one further biomarker. The invention also relates to tools for carrying out the aforementioned methods, such as diagnostic devices.

Cardiac disease, such as heart failure or coronary heart disease, heart attack is one of the leading causes of death for men and women in the US and in developed countries.

For example, heart failure is a severe problem in modern medicine. The impaired function of the heart can give rise to life-threatening conditions and results in discomfort for the patients suffering from heart failure. Heart failure can affect the right or the left heart ventricle, respectively, and can vary in strength. A classification system was originally developed by the New York Heart Association (NYHA). According to the classification system, the mild cases of heart failure are categorized as class I cases. These patients only show symptoms under extreme exercise. The intermediate cases show more pronounced symptoms already under less exercise (classes II and III) while class IV, shows already symptoms at rest (New York Heart Association. Diseases of the heart and blood vessels. Nomenclature and criteria for diagnosis, 6th ed. Boston: Little, Brown and co, 1964:114).

The prevalence of heart failure steadily increases in the population of the western developed countries over the last years. One reason for said increase can be seen in an increased average life expectation and improved survival after myocardial infarction due to modern medicine. The mortality rate caused by heart failure, however, could be further reduced by improved diagnostic and therapeutic approaches. The so-called “Framingham” study reported a reduction of the 5 year mortality from 70% to 59% in men and from 57% to 45% in women when comparing a time window of 1950 to 1969 with 1990 to 1999 (Fox C. S. et al., Circulation 110, 522-527, 2004). The “Mayo” study shows a reduction from 65% to 50% for men for a time window of 1996 to 2000 compared to 1979 to 1984 and from 51% to 46% for women (Roger V L, Weston S A, Redfield M M, et al., JAMA-J. Am. Med. Assoc. 292, 344-350, 2004). Notwithstanding this reduction of the mortality rate, the overall mortality due to heart failure is still a major burden to societies. One-year mortality for NYHA class II to III patients under ACE inhibitor therapy is still between 9-12% (SOLVD study; The SOLVD Investigators, NEJM 325, 293-302, 1991; The SOLVD Investigators, New Engl. J. Med. 327, 685-691, 1992) and for NYHA class IV without ACE inhibitor therapy 52% (Consensus study; The Consensus Trial Study Group, New Engl. J. Med. 316, 1429-1435, 1987).

Diagnosis of heart failure today relies predominantly on clinical symptoms, imaging modalities and the biomarkers, brain natriuretic peptide (BNP) and amino-terminal pro-brain natriuretic peptide (NT-proBNP). However, despite a wide acceptance of biomarkers in the medical community, the clinical utility of the gold standards BNP and NT-proBNP is limited by their lack of sensitivity in early stages of heart failure (see Rodeheffer, R. J., J. Am. Coll. Cardiol. 44, 740-749, 2004). In addition, BNP and/or NT-proBNP concentrations are influenced by confounding factors such as gender, age, BMI, or renal function [Balion C. et al. 2013. AHRQ Comparative Effectiveness Reviews. Agency for Healthcare Research and Quality (US), Rockville (Md.)]. This represents a huge medical problem since many patients progress through an asymptomatic phase of left ventricular systolic dysfunction (ALVSD) before the development of overt symptomatic disease. Because pharmacotherapy in asymptomatic patients was shown to positively affect clinical outcome, a reliable diagnosis of treatable early stages and in consequence, guideline-driven treatment is expected to reduce morbidity and mortality and have a high health economic impact. While there is little doubt within the medical community about the need for a cost effective screening for ALSVD (patients at risk to develop symptomatic heart failure), prohibitive costs and/or suboptimal clinical performance of the available diagnostic tests have so far prevented any screening program from being advocated by medical guidelines.

Recently, metabolic biomarkers for diagnosing heart failure and/or for monitoring heart failure progression or regression have been reported (see WO2011/092285 and WO2013/014286, respectively).

It is a goal of modern medicine to reliably identify and treat patients with heart failure, in particular with early stages of heart failure. Accordingly, means and methods for reliably diagnosing heart failure are still highly desired.

Therefore, the present invention relates to a method for diagnosing cardiac disease in a subject comprising the steps of:

a) determining in a sample of a subject the amounts of at least three lipid metabolite biomarkers and of at least one additional cardiac biomarker; and

b) comparing the amounts of the said biomarkers as determined in step a) to a reference (or references, in particular a reference for each of the biomarkers), whereby cardiac disease is to be diagnosed.

Thus, the present invention is based on the determination of the amounts at least three lipid metabolite biomarkers and of the amount(s) of at least one additional cardiac biomarker. Preferred lipid metabolite biomarkers are disclosed elsewhere herein.

The method as referred to in accordance with the present invention includes a method which essentially consists of the aforementioned steps or a method which includes further steps. However, it is to be understood that the method, in a preferred embodiment, is a method carried out ex vivo, i.e. not practised on the human or animal body. The method, preferably, can be assisted by automation.

The term “diagnosing” as used herein refers to assessing whether a subject suffers from a cardiac disease, or not. Accordingly, the presence or the absence of a cardiac disease in the subject can be diagnosed. Preferably, the term refers to ruling in or ruling out a cardiac disease. As will be understood by those skilled in the art, such an assessment, although preferred to be, may usually not be correct for 100% of the investigated subjects. The term, however, requires that a statistically significant portion of subjects can be correctly assessed and, thus, diagnosed. Whether a portion is statistically significant can be determined without further ado by the person skilled in the art using various well known statistic evaluation tools, e.g., determination of confidence intervals, p-value determination, Student's t-test, Mann-Whitney test, etc. Details are found in Dowdy and Wearden, Statistics for Research, John Wiley & Sons, New York 1983. Preferred confidence intervals are at least 50%, at least 60%, at least 70%, at least 80%, at least 90% or at least 95%. The p-values are, preferably, 0.2 or lower, 0.1 or lower, or 0.05 or lower. Preferably, the diagnosis is based on the biomarkers to be determined in the method of the present invention, i.e. on the at least three lipid metabolite biomarkers as specifically referred to in step a) of the method of the present invention and, if determined, on NT-proBNP or BNP and of at least one additional cardiac biomarker.

The term “diagnosing” includes individual diagnosis of a cardiac disease or its symptoms as well as continuous monitoring of a patient. Monitoring, i.e. diagnosing the presence or absence of a cardiac disease or the symptoms accompanying it at various time points, includes monitoring of patients known to suffer from a cardiac disease as well as monitoring of subjects known to be at risk of developing a cardiac disease. Furthermore, monitoring can also be used to determine whether a patient is treated successfully or whether at least symptoms of a cardiac disease can be ameliorated over time by a certain therapy. Moreover, monitoring may be used for active patient management including deciding on hospitalization, intensive care measures and/or additional qualitative monitoring as well as quantitative monitoring measures, i.e. monitoring frequency.

The term “diagnosing” further includes the prediction of a cardiac disease as referred to herein. Thus, the risk of the subject to suffer from cardiac disease is predicted.

In an embodiment, the presence of the disease in the test subject could be diagnosed. In another embodiment, the absence of the disease in the test subject could be diagnosed.

The term “cardiac disease” as used herein preferably refers to any disease involving the heart or blood vessels (vascular disease). In a preferred embodiment the cardiac disease is a vascular disease. Preferably, the vascular disease is peripheral artery disease and, more preferably, coronary artery disease. Also preferably, the vascular disease is atherosclerosis.

In another preferred embodiment, the cardiac disease is a disease which involves the heart. Preferably, the disease which involves the heart is selected from the group consisting of cardiomyopathy, heart failure, and pulmonary heart disease.

In another embodiment, the cardiac disease is a chronic cardiac disease, preferably a vascular disease. Thus, it is in particular envisaged that the cardiac disease is not an acute disease such as a myocardial infarction or stroke.

In a very preferred embodiment, the cardiac disease is coronary artery disease.

In another very preferred embodiment, the cardiac disease is heart failure.

In addition, it is envisaged that the cardiac disease is a combination of the aforementioned cardiac diseases. In particular, the cardiac disease to be diagnosed is heart failure accompanied by coronary artery disease (preferably, heart failure accompanied by coronary artery disease).

The term “heart failure” is well known in the art. As used herein relates to an impaired function of the heart. It is a progressive disorder in which the heart fails to pump oxygenated blood at a rate sufficient to meet the metabolic needs of the tissues. Preferably, the term heart failure as used herein relates to congestive heart failure (CHF).

Heart failure is the final common stage of many cardiovascular diseases and is defined as a clinical syndrome in which patients in the final stage show typical signs and symptoms of effort intolerance and/or fluid retention resulting from an abnormality of cardiac structure or function. As outlined further herein below, the term “heart failure” in the context of the method of the present invention encompasses both symptomatic forms and asymptomatic forms of heart failure. E.g. asymptomatic left ventricular systolic dysfunction or asymptomatic diastolic dysfunction may be diagnosed.

The impaired function of the heart can be a systolic dysfunction resulting in a significantly reduced ejection fraction of blood from the heart and, thus, a reduced blood flow. Specifically, systolic heart failure is characterized by a significantly reduced left ventricular ejection fraction (LVEF), preferably, an ejection fraction of less than 50% (heart failure with reduced ejection fraction, HFrEF). Alternatively, the impairment can be a diastolic dysfunction, i.e. a failure of the ventricle to properly relax. The latter is usually accompanied by a stiffer ventricular wall. The diastolic dysfunction causes inadequate filling of the ventricle and, therefore, results in consequences for the blood flow, in general. Thus, diastolic dysfunction also results in elevated enddiastolic pressures, and the end result is comparable to the case of systolic dysfunction (pulmonary edema in left heart failure, peripheral edema in right heart failure.) Heart failure may, thus, affect the right heart (pulmonary circulation), the left heart (body circulation) or both. Techniques for measuring an impaired heart function and, thus, heart failure, are well known in the art and include echocardiography, electrophysiology, angiography. It will be understood that the impaired function of the heart can occur permanently or only under certain stress or exercise conditions. Dependent on the strength of the symptoms, heart failure can be classified as set forth elsewhere herein. Typical symptoms of heart failure include dyspnea, chest pain, dizziness, confusion, pulmonary and/or peripheral edema. It will be understood that the occurrence of the symptoms as well as their severity may depend on the severity of heart failure and the characteristics and causes of the heart failure, systolic or diastolic or restrictive i.e. right or left heart located heart failure. Further symptoms of heart failure are well known in the art and are described in the standard text books of medicine, such as Stedman or Brunnwald.

Two subsets of heart failure with different pathophysiology are described based on a measurement of left ventricular ejection fraction (LVEF), which is the percentage of the total amount of blood in the left ventricle that is pushed out with each heartbeat: Heart failure with reduced left ventricular ejection fraction (HFrEF) and heart failure with preserved left ventricular ejection fraction (HFpEF). Preferably, the heart failure to be diagnosed in the accordance with the present invention is HFpEF, more preferably the heart failure is HFrEF.

HFrEF, also known as systolic heart failure, is characterized by reduced heart muscle contraction and emptying of the left ventricle. The expression “reduced left ventricular ejection fraction”, preferably, relates to a left ventricular ejection fraction (LVEF) of lower than 50%. Moreover it is envisaged that the LVEF is lower than 40%, in particular lower than 35%. Also the left ventricular ejection fraction may be lower than 50% but larger than 35%.

While there are many causes of HFrEF, the most common is related to ischemic cardiomyopathy resulting from coronary artery disease and prior myocardial infarctions. Ischemic cardiomyopathy (ICMP) occurs when narrowed or blocked coronary arteries restrict blood flow and oxygen supply to the heart tissue, damaging or weakening the heart muscle (loss of functional myocardium). One of the most common non-ischemic causes of HFrEF is dilatative cardiomyopathy (DCMP, also referred to as dilated cardiomyopathy). DCMP is a condition in which the heart's ability to pump blood is decreased because the heart's main pumping chamber, the left ventricle, is enlarged, dilated and weak. The cause for DCMP can range from heart muscle infection (myocarditis), high blood pressure, heart valve disease, and alcohol abuse to familial (hereditary) forms. Preferably, the terms NICMP (non-ischemic cardiomyopathy) and DCMP are used interchangeably herein.

A subject who suffers from HFrEF may, thus, suffer from dilatative cardiomyopathy (DCMP, frequently also referred to as dilated cardiomyopathy) or ischemic cardiomyopathy (ICMP).

Accordingly, the term HFrEF preferably encompasses ICMP and DCMP. Thus, the HFrEF to be diagnosed may be DCMP, or in particular ICMP. Preferred marker combinations for HFrEF, ICMP and DCMP are disclosed elsewhere herein.

A subject who suffers from ICMP, preferably, has a reduced LVEF and more than 50% coronary stenosis.

A subject who suffers from DCMP, preferably, has a reduced LVEF and less than 50% coronary stenosis. In particular, a subject who suffers from DCMP, preferably, has a reduced LVEF, less than 50% coronary stenosis and a left ventricular end diastolic diameter of larger than 55 mm.

Preferred combinations of at least three lipid metabolite biomarkers (that can be used in combination with the at least one additional cardiac biomarker) for diagnosing HFrEF, or the subforms of HFrEF (DCMP or ICMP) are disclosed elsewhere herein.

HFrEF in accordance with the method of the present invention may be symptomic or asymptomatic. Accordingly, the present invention preferably allows for the the diagnosis of symptomic or asymptomatic systolic dysfunction.

As set forth above, the heart failure to be diagnosed may be heart failure with preserved left ventricular ejection fraction (HFpEF) also known as diastolic heart failure. The term “preserved left ventricular ejection fraction” preferably refers to a LVEF of larger than 50%. Also envisaged is a LVEF of larger than 60%. Alternatively, the term “preserved left vetricular ejection fraction” preferably refers to a LVEF of larger than 55%. HFpEF is characterized by a disturbed relaxation and dilatation of the left ventricle. Diastolic left ventricular dysfunction results from LV hypertrophy and cardiac fibrosis resulting in increased myocardial stiffness. The muscle becomes stiff and loses some of its ability to relax, especially during diastole. As a result, the affected chamber has trouble filling with blood during diastole, leading to an elevated left ventricular filling pressure. HFpEF in accordance with the method of the present invention may be symptomic or asymptomatic. Accordingly, the present invention preferably allows for the the diagnosis of symptomic or asymptomatic diastolic dysfunction.

A subject suffering from HFpEF preferably has a cardium septum thickness of larger than 12 mm.

Alternatively, the subject may have a cardium septum thickness of larger than 11 mm.

In a preferred embodiment, the cardiac disease is HFpEF. HFpEF can be symptomatic or asymptomatic.

Preferred combinations of at least three lipid metabolite biomarkers (that can be used in combination with the at least one additional cardiac biomarker) for diagnosing HFpEF are disclosed elsewhere herein.

Moreover, heart failure as used herein relates to symptomatic or asymptomatic heart failure. Accordingly, the method of the present invention allows for diagnosing symptomatic heart failure and, in particular, asymptomatic heart failure. Thus, the present invention, in particular, allows for diagnosing symptomatic or asymptomic CHF, HFrEF, DCMP, ICMP and/or HFpEF.

Symptoms of heart failure are well known in the art and are described above. Asymptomatic heart failure is preferably heart failure according to NYHA class I. A subject with heart failure according to NYHA class I has no limitation of physical activity and ordinary physical activity does not cause undue breathlessness, fatigue, or palpitations. Symptomatic heart failure is preferably heart failure according to NYHA class II, III and/or VI, in particular according to NYHA class II and/or III. A subject with heart failure according to NYHA class II or III has a slight (NYHA class II) or marked (NYHA class III) limitation of physical activity, is comfortable at rest but ordinary (NYHA class II) or less than ordinary (NYHA class III) physical activity results in undue breathlessness, fatigue, or palpitations.

As described herein below in more detail, the subject to be tested preferably shows symptoms of heart failure. In this case, it is in particular diagnosed whether the subject suffers from symptomatic heart failure (or symptomatic HFrEF, DCMP, ICMP or HFpEF). More preferably, the subject does not show symptoms of heart failure. In this case asymptomatic heart failure is diagnosed. In this case, it is in particular diagnosed whether the subject suffers from asymptomatic heart failure (or asymptomatic HFrEF, DCMP, ICMP or HFpEF).

The method of the present invention envisages the determination of the amount of at least three lipid metabolite biomarkers and of at least one additional cardiac biomarker, i.e. of a combination of at least three lipid metabolite biomarkers and of least one additional cardiac biomarker. Preferred combinations are described elsewhere herein.

The term “lipid metabolite biomarker” as used herein refers to a molecular species which serves as an indicator for a disease or effect as referred to in this specification. Said molecular species can be a metabolite itself which is found in a sample of a subject. Moreover, in certain cases the lipid metabolite biomarker may also be a molecular species which is derived from said metabolite. In such a case, the actual metabolite will be chemically modified in the sample or during the determination process and, as a result of said modification, a chemically different molecular species, i.e. the analyte, will be the determined molecular species. It is to be understood that in such a case, the analyte represents the actual metabolite and has the same potential as an indicator for the respective medical condition, i.e. for a cardiac disease. In an embodiment of the present invention the triacylglyceride(s) will be determined as such, as disclosed elsewhere herein. In an embodiment of the present invention the phosphatidylcholine(s) will be determined as such, as disclosed elsewhere herein. In an embodiment of the present invention the ceramide(s) will be determined as such, as disclosed elsewhere herein. In an embodiment of the present invention the sphingomyelin(s) will be determined as such, as disclosed elsewhere herein. The term “metabolite” is well known in art. Preferably, the metabolite in accordance with the present invention is a small molecule compound.

In the method according to the present invention at least the amounts of at least three lipid metabolite biomarkers shall be determined. The term “at least three lipid metabolite biomarkers” as used herein, means three or more than three. Accordingly, the amounts of three, four, five, six, seven, eight, nine, ten or even more lipid metabolite biomarkers may be determined (and compared to a reference). Preferably, the amounts of three to ten lipid metabolite biomarkers are determined (and compared to a reference).

The at least three lipid metabolite biomarkers to be determined are selected from the group consisting of at least one sphingomyelin biomarker, at least one triacylglyceride biomarker, at least one cholesterylester biomarker, at least one phosphatidylcholine biomarker, and at least one ceramide biomarker. Thus, the method of the present invention envisages (in addition to the determination of at least cardiac biomarker) the determination of one or more sphingomyelin biomarker, one or more triacylglyceride biomarker, one or more cholesterylesters biomarker, one or more phosphatidylcholine biomarker, and/or one or more ceramide biomarker. Furthermore, the amount of glutamic acid may be determined. In an embodiment, the amounts of i) at least one one sphingomyelin biomarker, ii) at least one triacylglyceride biomarker, and iii) at least one cholesterylester biomarker or at least one phosphatidylcholine biomarker are determined.

The at least three lipid metabolite biomarkers to be determined preferably belong to at least three of the different compound classes referred to above. For example, one of the at least three lipid metabolite biomarkers is a triacylglycerid biomarker, one is a sphingomyelin biomarker, and one is a cholesterylester biomarker. However it is also envisaged that the at least three lipid metabolite biomarkers belong to a single compound class, or to two compound classes.

Preferred lipid metabolite biomarkers to be determined in accordance with the present invention are disclosed in column 1 of Table 1 of the examples section.

Thus, the at least one triacylglycerid biomarker is preferably selected from the group consisting of OSS2, SOP2, SPP1, SSP2, SSS, PPO1, and PPP, more preferably selected from the group consisting of OSS2, SOP2, SPP1, SSP2, PPO1 and PPP, even more preferably selected from OSS2, SOP2, SPP1, SSP2 and PPO1, in particular OSS2 and SOP2, and most preferably OSS2. Preferably, one, two or three, or more triacylglyceride biomarker are determined.

The at least one sphingomyelin biomarker is preferably selected from the group consisting of SM10, SM18, SM2, SM21, SM23, SM24, SM28, SM29, SM3, SM5, SM8, and SM9 (in particular from SM10, SM18, SM21, SM23, SM24 and SM28). More preferably, the at least one sphingomyelin biomarker is selected from the group consisting of SM23, SM24, and SM18. Even more preferably, the at least one sphingomyelin biomarker is SM18 or SM24, and most preferably SM23. Preferably, one, two, three, four or more sphingomyelin biomarker are determined.

The at least one ceramide biomarker is preferably selected from the group consisting of Cer(d16:1/24:0), Cer(d17:1/24:0), Cer(d18:1/23:0), Cer(d18:1/24:1), and Cer(d18:2/24:0). Preferably, the amounts of one or two, or more ceramide biomarker are determined. More preferably, the at least one ceramide biomarker is selected from the group consisting of Cer(d16:1/24:0), and Cer(d18:1/24:1). Even more preferably, the at least one ceramide biomarker is Cer(d16:1/24:0).

The at least one phosphatidylcholine biomarker is preferably PC4 or PC8. Preferably, the amount of one phosphatidylcholine biomarker is determined, in particular PC4. However, it is also envisaged to determine both biomarker PC4 and PC8.

The at least one cholesterylester biomarker is preferably cholesterylester C18:0 or cholesterylester C18:2. Preferably, the amount of one cholesterylester biomarker is determined, in particular cholesterylester 018:2. However, it also envisaged to determine both cholesterylester C18:0 and cholesterylester C18:2. Preferably, the cholesterylester biomarker is not cholesterylester C18:1.

Accordingly, the at least three lipid metabolite biomarkers as referred to in step a) of the method of the present invention are preferably selected from the group of biomarkers consisting of OSS2, SOP2, SPP1, SSP2, SSS, PPO1, PPP, SM10, SM18, SM2, SM21, SM23, SM24, SM28, SM29, SM3, SM5, SM8, SM9, Cer(d16:1/24:0), Cer(d17:1/24:0), Cer(d18:1/23:0), Cer(d18:1/24:1), Cer(d18:2/24:0), PC4, PC8, cholesterylester C18:0 cholesterylester C18:2, and glutamic acid. The aforementioned biomarkers are metabolite biomarkers.

In addition to the at least three lipid metabolite biomarkers, the amount of at least one additional cardiac biomarker shall be determined. The term “cardiac biomarker” as used herein preferably refers to a marker that is increased or decreased in conjunction with a cardiac disease. Preferably, the at least one additional cardiac biomarker is selected from the biomarkers shown in Table 3 of the Examples section. More preferably, the at least one cardiac biomarker is selected from the group consisting of at least one general lipid cardiac biomarker, at least one lipoprotein subfraction biomarker, at least one apolipoprotein biomarker and at least one inflammation biomarker.

Preferably, the at least one general lipid cardiac biomarker is selected from the group consisting of total cholesterol, HDL-cholesterol (High Density Lipoprotein Cholesterol), triglycerides, LDL-cholesterol (Low Density Lipoprotein Cholesterol), a ratio of total cholesterol and HDL-cholesterol (in particular the ratio of total cholesterol to HDL-cholesterol), and non-HDL cholesterol.

The at least one general lipid cardiac biomarkers are preferably determined in a blood, serum or plasma sample, in particular in a serum or plasma sample.

In an embodiment, the at least one general lipid cardiac biomarker is total cholesterol. Cholesterol is a lipid sterol that is produced in and transported throughout the bloodstream in eukaryotes. Cholesterol is a critical compound used in the structure of cell membranes, hormones, and cell signaling. It is an essential component of animal cell structure in order to maintain permeability and fluidity. Because high blood cholesterol has been associated with hardening of the arteries (atherosclerosis), heart disease, and a raised risk of death from heart attacks, cholesterol testing is considered a routine part of preventive health care. The term “total cholesterol” preferably refers to the total amount of cholesterol in the sample. Total cholesterol preferably includes the cholesterol as well as cholesteryl esters bound to carrier molecules such as lipoproteins, free cholesterol, and cholesteryl esters.

In an embodiment, the amount of triglycerides are determined as at least one general lipid cardiac biomarker, i.e. the sum of the amounts of triglycerides present the sample. Thus, the term “triglycerides” preferably refers to the amount of the entirety of triglycerides present in the sample to be investigated.

In an embodiment, the at least one general lipid cardiac biomarker is LDL-cholesterol. The term “LDL cholesterol” is well known in the art and means low density lipoprotein cholesterol.

In an embodiment, the at least one general lipid cardiac biomarker is HDL-cholesterol. The term “HDL cholesterol” is well known in the art and means high density lipoprotein cholesterol.

HDL and LDL cholesterol levels in the blood are important indicators of many disease states. High blood levels of LDLs are associated with health problems and cardiovascular disease. LDL particles that accumulate within arteries can form plaques over time, which can increase chances of a stroke, heart attack, or vascular disease. HDL particles are able to remove cholesterol from within arteries and transport it back to the liver for re-utilization or excretion.

In an embodiment, the at least one general lipid cardiac biomarker is a ratio of total cholesterol and HDL-cholesterol. Preferably, the ratio of the amount of total cholesterol to the amount of HDL-cholesterol is determined (Chol/HDLC Ratio). In the context of the present invention, the ratio is also considered as biomarker. For determining this biomarker (i.e. the ratio) the amounts of total cholesterol and HDL-cholesterol are determined and the ratio is calculated. An optimal Chol/HDLC Ratio is less than 3.5, preferably less than 3.5 but equal to or higher than 1. A higher ratio means a higher risk of cardiac disease.

In an embodiment, the ratio is compared to a reference ratio. In another embodiment, the ratio contributes to a score (as described elsewhere) which is compared to a reference score.

In an embodiment, the at least one cardiac biomarker is non-HDL cholesterol. The amount of non-HDL cholesterol is preferably the amount of total cholesterol minus the amount of HDL cholesterol in the sample. The the amount of HDL-cholesterol is subtracted from the amount of total cholesterol present in the sample. An optimal level of non-HDL cholesterol is less than 130 milligrams per deciliter (mg/dL), or 3.37 millimoles per liter (mmol/L). Higher amounts mean a higher risk of heart disease.

Preferably, at least one general lipid cardiac biomarker is determined in the context of the present invention. More preferably, a combination of the at least one general lipid cardiac biomarker is determined. In a preferred embodiment at least the amount of total cholesterol and triglycerides are determined. In another preferred embodiment, at least the amounts of total cholesterol, triglycerides, HDL cholesterol and LDL cholesterol are determined. Further, it is envisaged that the amounts of total cholesterol, triglycerides, HDL-cholesterol and LDL cholesterol and Non-HDL cholesterol are determined. Also it is envisaged that the amounts of total cholesterol, triglycerides, HDL-cholesterol and LDL cholesterol and Non-HDL cholesterol as well as the ratio Chol/HDLC are determined. In addition, all general lipid cardiac biomarkers can be determined.

In a preferred embodiment the the amount(s) HDL cholesterol and/or LDL cholesterol is (are) determined. In particular, both the amounts of HDL cholesterol and LDL cholesterol are determined.

Thus, it is in particular envisaged to determine the amount of HDL cholesterol and/or the amount of LDL cholesterol as a cardiac biomarker. The most preferred additional cardiac biomarker is HDL cholesterol. Further, it is envisaged to determine the amounts of both HDL cholesterol and LDL cholesterol.

Preferably, the at least one lipoprotein subfraction biomarker is selected from LDL particles (herein also referred to as total LDL particles), small LDL particles, medium LDL particles and large HDL particles. Preferably, the amount, or the number of the said particules is determined. Thus, the number or, in particular, the amount of all LDL particles present in the sample, of small LDL particles, of medium LDL particles and/or of large HDL particles is determined.

In an embodiment, the amount or number of total LDL particles is determined as lipoprotein subfraction biomarker, herein also referred to as total amount or total number of LDL particles. Preferably, an increased number or amount of total LDL particles as compared to the refence is associated with the presence of the cardiac disease or the risk for the disease, whereas reduced number or amount as compared to the reference is indicative for the absence of the cardiac disease or for the absence of a risk thereof.

In an embodiment, the amount or number of small LDL particles is determined as lipoprotein subfraction biomarker. Preferably, an increased number or amount of small LDL particles as compared to the refence is assicioated with the presence of the cardiac disease or the risk for the disease, whereas reduced number or amount as compared to the reference is indicative for the absence of the cardiac disease or for the absence of a risk thereof.

In an embodiment, the amount or number of medium LDL particles is determined as lipoprotein subfraction biomarker. Preferably, an increased number or amount of medium LDL particles as compared to the refence is assicioated with the presense of the cardiac disease or the risk for the disease, whereas reduced number or amount as compared to the reference is indicative for the absence of the cardiac disease or for the absence of a risk thereof.

In an embodiment, the amount or number of large HDL particles is determined as lipoprotein subfraction biomarker. Preferably, a reduced number or amount of large HDL particles as compared to the refence is assicioated with the presence of the cardiac disease or the risk for the disease, whereas an increased number or amount as compared to the reference is indicative for the absence of the cardiac disease or for the absence of a risk thereof.

Low-density lipoprotein particle (LDL-P) testing, also referred to LDL subfraction testing evaluates LDL particles according to their number, size, density, and/or electrical charge. It may offer useful information for assessing risk in people who have a personal or family history of heart disease at a young age. The lipoprotein subfraction biomarkers are well known in the art and e.g. described by Caulfield (see Clinical Chemistry August 2008 vol. 54 no. 8 1307-1316), Blanche et al. Biochim Biophys Acta 1981; 665:408-19, and Berneis J Lipid Res 2002; 43:1363-79. The preferred sizes of the small LDL particles, medium LDL particles and large HDL particles are listed in Table 1 of Caulfield. The size is preferably the diameter.

Large HDL particles preferably have a size of about 105 to about 145 Angstrom; Medium LDL particles preferably have a size of about 211 to about 219.9 Angstrom;

Small LDL particles preferably have a size of about 201.7 to about 211 Angstrom;

1 Angstrom is 0.1 nm.

Preferably, at least one lipoprotein subfraction biomarker is determined. More preferably, two or three, or in particular all subfraction biomarkers are determined. For example, it is envisaged to determine the following biomarkers: number or amount of small LDL particles, medium LDL particles and large HDL particles.

Preferably, the at least one apolipoprotein biomarker is selected from apolipoprotein B and lipoprotein(a).

In an embodiment, the at least one apolipoprotein biomarker is apolipoprotein B. Apolipoprotein B (ApoB) is a protein that in humans is encoded by the APOB gene. It is the primary apolipoprotein of chylomicrons, VLDL, IDL, and LDL particles which is responsible for the transport of lipids. High levels of ApoB are related to heart disease.

In another embodiment, the at least one apolipoprotein biomarker is lipoprotein(a). Lipoprotein(a) (also called Lp(a) or LPA) is a lipoprotein subclass. The marker is well known in the art (see e.g. McCormic, McCormick SPA. Lipoprotein(a): Biology and Clinical Importance. The Clinical Biochemist Reviews. 2004; 25(1):69-80). Lipoprotein(a) is a unique lipoprotein that has emerged as an independent risk factor for developing vascular disease.

Preferably, the amount of one apolipoprotein biomarker is determined in the context of the present invention, and thus either ApoB or Lp(a). More preferably, the amounts of both biomarkers are determined.

Preferably, the at least one inflammation biomarker is selected from C-reactive protein (CRP), in particular high sensitivity C-reactive protein (hsCRP) and Lipoprotein-associated phospholipase A2 (Lp-PLA2).

In an embodiment, the at least one inflammation biomarker is C-reactive protein (CRP), in particular high sensitivity C-reactive protein (hsCRP). The biomarker is well known in the art. CRP is a protein that increases in the blood with inflammation. Studies have suggested that a persistent low level of inflammation plays a major role in atherosclerosis, the narrowing of blood vessels due to build-up of cholesterol and other lipids, which is often associated with CVD. Preferably, a highly sensitive test is applied is capable of measuring low concentrations of the biomarker in the sample. The hsCRP test accurately measures low levels of C-reactive protein to identify low but persistent levels of inflammation and thus helps predict a person's risk of developing cardiovascular disease.

In another embodiment, the inflammation biomarker is Lipoprotein-associated phospholipase A2 (Lp-PLA2). The biomarker is well known in the art. It is a phospholipase A2 enzyme that in humans is encoded by the PLA2G7 gene. Lp-PLA2 is a 45-kDa protein of 441 amino acids. The marker promotes localized inflammation, which is involved in the formation of ruptureprone plaque. Plaque rupture is a major cause of both stroke and heart attack. Elevated Lp-PLA2 levels appear to be predictive of future coronary events, independent of other cardiac risk factors. Lp-PLA2 has minimal biological variation and is not elevated in systemic inflammatory conditions, providing consistent, reliable results.

Preferably, the amount of one inflammation biomarker is determined in the context of the present invention, and thus either CRP or Lp-PLA2. More preferably, the amounts of both inflammation biomarkers are determined.

Any additional cardiac biomarker can be determined in addition to the at least the lipid metabolite biomarkers. Preferably, the at least one additional cardial biomarker is selected from the general lipid cardiac biomarkers, more preferably the at least one additional cardial biomarker is selected from the lipoprotein subfraction biomarkers, even more preferably the at least one additional cardial biomarker is selected from the apolipoprotein biomarkers, most preferably, the at least one additional cardial biomarker is selected from the inflammation biomarkers.

In the context of the present invention, the at least one additional cardiac biomarker is not a natriuretic peptide, in particular not NT-proBNP or BNP. As described elsewhere herein, the NT-proBNP or BNP can be determined in addition to the at least three lipid metabolite biomarkers and to the at least one additional cardiac biomarker.

For the prediction of the risk to suffer from cardiac disease, the amounts of at least the three lipid metabolite biomarkers, of small LDL particles, triglycerides, HDL cholesterol and large HDL particles are determined in combination.

Lipid Metabolite Biomarker Definitions

In accordance with the present invention, the amounts of at least three lipid metabolite biomarkers selected from the group consisting of OSS2, SOP2, SPP1, SSP2, SSS, PPO1, PPP, SM10, SM18, SM2, SM21, SM23, SM24, SM28, SM29, SM3, SM5, SM8, SM9, Cer(d16:1/24:0), Cer(d17:1/24:0), Cer(d18:1/23:0), Cer(d18:1/24:1), Cer(d18:2/24:0), PC4, PC8, cholesterylester C18:0, cholesterylester C18:2 and glutamic acid shall be determined (in addition to the at least one cardiac biomarker).

A preferred definition of these lipid metabolite biomarkers is provided in Table 1 of the Examples section. Accordingly, the lipid metabolite biomarkers are preferably defined as follows:

Triacylglyceride (TAG) Biomarkers

The biomarkers OSS2, SOP2, SPP1, SSP2, SSS, PPO1, and PPP are triacylglycerides (TAG: triacylglyceride).
OSS2 is TAG(C18:1, C18:0, C18:0)
PPO1 is TAG(C16:0, C16:0, C18:1)
PPP is TAG(C16:0, C16:0, C16:0)
SOP2 is TAG(C18:0, C18:1, C16:0)
SPP1 is TAG(C18:0, C16:0, C16:0)
SSP2 is TAG(C18:0, C18:0, C16:0)
SSS is TAG(C18:0, C18:0, C18:0)

The triacylglyceride TAG(Cx¹:y¹, Cx²:y², Cx³:y³) is preferably denoted to mean that the triacylglyceride comprises three fatty acid ester residues, wherein one fatty acid ester residue is Cx¹:y¹ which means that this residue comprises x¹ carbon atoms and y¹ double bonds, wherein one fatty acid ester residue is Cx²:y² which means that this residue comprises x² carbon atoms and y² double bonds, and wherein one fatty acid ester residue is Cx³:y³ which means that this residue comprises x³ carbon atoms and y³ double bonds. Preferably, any of these fatty acid ester residues may be attached to any former hydroxyl groups of the glycerol.

For example, SOP2 comprises three fatty acid ester residues, wherein one fatty acid ester residue is C18:0 which means that this residue comprises 18 carbon atoms and 0 double bonds, wherein one fatty acid ester residue is C18:1 which means that this residue comprises 18 carbon atoms and 1 double bond, and wherein one fatty acid ester residue is C16:0 which means that this residue comprises 16 carbon atoms and 0 double bonds. Preferably, any of these fatty acid ester residues may be attached to any former hydroxyl groups of the glycerol.

Ceramide (CER) Biomarkers

The biomarkers Cer(d16:1/24:0), Cer(d17:1/24:0), Cer(d18:1/23:0), Cer(d18:1/24:1), and Cer(d18:2/24:0) are ceramides.

The ceramide CER(dx¹:y¹/x²:y²) is preferably denoted to mean that the ceramide comprises the “sphingosine-backbone” dx¹:y¹, wherein x¹ denotes the number of carbon atoms and y¹ the number of double bonds, and a “fatty acid amid” residue x²:y², wherein x² denotes the number of carbon atoms and y² the number of double bonds thereof. Preferably, “d” indicates that the backbone comprises two hydroxyl groups.
Cer(d16:1/24:0) is Ceramide (d16:1/24:0)
Cer(d17:1/24:0) is Ceramide (d17:1124:0)
Cer(d18:1/23:0) is Ceramide (d18:1/23:0)
Cer(d18:1/24:1) is Ceramide (d18:1/24:1)
Cer(d18:2/24:0) is Ceramide (d18:2/24:0)

For example, the ceramide Cer(d16:1/24:0) is preferably denoted to mean that the ceramide comprises the “sphingosine-backbone” d16:1, comprising 16 carbon atoms and 1 double bond, and a “fatty acid amid” residue 24:0 comprising 24 carbon atoms and 0 double bonds.

Sphingomyelin (SM) Biomarkers

SM is the abbreviation for Sphingomyelin. As set forth herein below, the amounts of the sphingomyelin biomarkers SM10, SM18, SM2, SM21, SM23, SM24, SM5, and SM9 may be determined by determining the amount of a single sphingomyelin species in the sample, or by determining the total amount of two (or even three) sphingomyelin species which have the same or essentially the same molecular weight (see Table 1 in the Examples section). For example, for SM10 the amount of Sphingomyelin(d18:1/18:0) can be determined, or the total amount of Sphingomyelin(d18:1/18:0) and Sphingomyelin(d16:1/20:0). Whether the amount of a single species or of two (or three) species is determined may depend on the assay used for the determination. For example, the amount of a single species is determined, if the assay underlying the determination step (step a.) allows for the determination of a single species. The total amount of two (or three) species may be determined, if the assay underlying the determination step allows for the determination of the total amount of the two (or three) species only (rather than for the determination of the single species), in other words the assay is not capable of differentially determining the amounts of each of the two (or three) species. E.g. if the determination of the amount of a biomarker comprises mass spectrometry, the determination of the amount of the biomarker is preferably based on a single peak in a mass spectrum for the two (or three) species since the peaks of the species overlap. This is taken into account by the skilled person.

In Table 1 of the Examples section, the preferred Sphingomyelin species for SM10, SM18, SM2, SM21, SM23, SM24, SM5, and SM9 are listed (the species are referred to as “analyte” in this table).

For SM10, SM2, SM24, SM5, and SM9 two analytes are listed (analyte 1 and analyte 2). The said biomarkers preferably refer to Analyte 2 (for example, for SM2: SM(d16:1/16:0)), more preferably to Analyte 1 (for example, for SM2: SM(d18:1/14:0)) and most preferably to Analyte 1 and 2 (for SM2: Sphingomyelin(d18:1/14:0) and Sphingomyelin(d16:1/16:0)). Accordingly, a biomarker for which two species are listed in table 1 is Analyte 1, Analyte 2 or a combination of Analyte 1 and Analyte 2.

The amount of said biomarker is, thus, preferably determined by determining the amount of Analyte 2, or more preferably of Analyte 1 and most preferably by determining the combined (and thus the total) amount of Analyte 1 and Analyte 2. Accordingly, the amount of said biomarker is the amount of Analyte 1 or 2, or the sum of the amounts of Analyte 1 and 2. The same applies to PC8.

For the biomarkers SM18, SM21 and SM 23 three analytes are listed (Analyte 1, 2 and 3). The said biomarkers preferably refer to Analyte 3, more preferably to Analyte 2, even more preferably to Analyte 1, and most preferably to Analyte 1, 2 and 3. Accordingly, a biomarker for which three species are listed in Table 1 is Analyte 1, Analyte 2, or Analyte 3 or a combination of Analyte 1, Analyte 2 and Analyte 3.

If two analytes are listed for a biomarker (Analyte 1 and Analyte 2), it is envisaged that the biomarker is Analyte 1. Alternatively, the biomarker may be Analyte 2. If three analytes are listed for a biomarker (Analyte 1, Analyte 2 and Analyte 3), it is envisaged that that the biomarker is Analyte 1. Alternatively, the biomarker may be Analyte 2. Also, the biomarker may be Analyte 3. Alternatively the biomarker may be a combination of Analyte 1 and Analyte 2. Alternatively the biomarker may be a combination of Analyte 1 and Analyte 3. Alternatively the biomarker may be a combination of Analyte 2 and Analyte 3. Alternatively the biomarker may be a combination of Analyte 1, Analyte 2 and Analyte 3.

The amount of said biomarker is, thus, preferably determined by determining the amount of Analyte 3, or more preferably of Analyte 2, even more preferably of Analyte 1 and most preferably by determining the combined (and thus the total) amount of Analyte 1, Analyte 2, and Analyte 3. Accordingly, the amount of said biomarker is the amount of Analyte 1, 2, or 3, or the sum of the amounts of Analyte 1, 2 and 3.

The following applies in particular:

The biomarker SM10 preferably refers to Sphingomyelin(d18:1/18:0), or more preferably to Sphingomyelin(d18:1/18:0) and Sphingomyelin(d16:1/20:0). Accordingly, the biomarker SM10 is Sphingomyelin(d18:1/18:0), or a combination of Sphingomyelin(d18:1/18:0) and Sphingomyelin(d16:1/20:0).

The amount of SM10 is, thus, preferably determined by determining the amount of Sphingomyelin(d18:1/18:0), or by determining the combined (and thus the total) amount of Sphingomyelin(d18:1/18:0) and Sphingomyelin(d16:1/20:0). Accordingly, the amount of SM10 is the amount of Sphingomyelin(d18:1/18:0), or the sum of the amounts of Sphingomyelin(d18:1/18:0) and Sphingomyelin(d16:1/20:0).

The biomarker SM18 preferably refers to Sphingomyelin(d18:1/21:0), or more preferably to Sphingomyelin(d18:1/21:0), Sphingomyelin(d16:1/23:0) and Sphingomyelin(d17:1/22:0). Accordingly, the biomarker SM18 is Sphingomyelin(d18:1/21:0), or a combination of Sphingomyelin(d18:1/21:0), Sphingomyelin(d16:1/23:0) and Sphingomyelin(d17:1/22:0).

The amount of SM18 is, thus, preferably determined by determining the amount of Sphingomyelin(d18:1/21:0), or by determining the combined (and thus the total) amount of Sphingomyelin(d18:1/21:0) Sphingomyelin(d16:1/23:0) and Sphingomyelin(d17:1/22:0). Accordingly, the amount of SM18 is the amount of Sphingomyelin(d18:1/21:0), or the sum of the amounts of Sphingomyelin(d18:1/21:0), Sphingomyelin(d16:1/23:0) and Sphingomyelin(d17:1/22:0).

The biomarker SM2 preferably refers to Sphingomyelin(d18:1/14:0) or more preferably to Sphingomyelin(d18:1/14:0) and Sphingomyelin(d16:1/16:0). Accordingly, the biomarker SM2 is Sphingomyelin(d18:1/14:0), or a combination of Sphingomyelin(d18:1/14:0) and Sphingomyelin(d16:1/16:0).

The amount of SM2 is, thus, preferably determined by determining the amount of Sphingomyelin(d18:1/14:0), or by determining the combined (and thus the total) amount of Sphingomyelin(d18:1/14:0) and Sphingomyelin(d16:1/16:0). Accordingly, the amount of SM2 is the amount of Sphingomyelin(d18:1/14:0), or the sum of the amounts of Sphingomyelin(d18:1/14:0) and Sphingomyelin(d16:1/16:0).

The biomarker SM21 preferably refers to Sphingomyelin(d17:1/23:0) or more preferably to Sphingomyelin(d17:1/23:0), Sphingomyelin(d18:1/22:0) and Sphingomyelin(d16:1/24:0). Accordingly, the biomarker SM21 is Sphingomyelin(d17:1/23:0), or a combination of Sphingomyelin(d17:1/23:0), Sphingomyelin(d18:1/22:0), and Sphingomyelin(d16:1/24:0).

The amount of SM21 is, thus, preferably determined by determining the amount of Sphingomyelin(d17:1/23:0), or by determining the combined (and thus the total) amount of Sphingomyelin(d17:1/23:0), Sphingomyelin(d18:1/22:0) and Sphingomyelin(d16:1/24:0). Accordingly, the amount of SM21 is the amount of Sphingomyelin(d17:1/23:0), or the sum of the amounts of Sphingomyelin(d17:1/23:0), Sphingomyelin(d18:1/22:0) and Sphingomyelin(d16:1/24:0).

The biomarker SM23 preferably refers to Sphingomyelin(d18:1/23:1) or more preferably to Sphingomyelin(d18:1/23:1), Sphingomyelin(d18:2/23:0) and Sphingomyelin(d17:1/24:1). Accordingly, the biomarker SM23 is Sphingomyelin(d18:1/23:1), or a combination of Sphingomyelin(d18:1/23:1), Sphingomyelin(d18:2/23:0), and Sphingomyelin(d17:1/24:1). Further, in another embodiment, it is envisaged that SM23 is Sphingomyelin (d17:1124:1).

The amount of SM23 is, thus, preferably determined by determining the amount of Sphingomyelin(d18:1/23:1), or by determining the combined (and thus the total) amount of Sphingomyelin(d18:1/23:1), Sphingomyelin(d18:2/23:0) and Sphingomyelin(d17:1/24:1). Accordingly, the amount of SM23 is the amount of Sphingomyelin(d18:1/23:1), or the sum of the amounts of Sphingomyelin(d18:1/23:1), Sphingomyelin(d18:2/23:0), and Sphingomyelin(d17:1/24:1). Further, it is envisaged, in another embodiment, that the amount of SM23 is determined by determining the amount of Sphingomyelin (d17:1124:1).

The biomarker SM24 preferably refers to Sphingomyelin(d18:1/23:0) or more preferably to Sphingomyelin(d18:1/23:0) and Sphingomyelin(d17:1/24:0). Accordingly, the biomarker SM24 is Sphingomyelin(d18:1/23:0), or a combination of Sphingomyelin(d18:1/23:0) and Sphingomyelin(d17:1/24:0).

The amount of SM24 is, thus, preferably determined by determining the amount of Sphingomyelin(d18:1/23:0), or by determining the combined (and thus the total) amount of Sphingomyelin(d18:1/23:0) and Sphingomyelin(d17:1/24:0). Accordingly, the amount of SM24 is the amount of Sphingomyelin(d18:1/23:0), or the sum of the amounts of Sphingomyelin(d18:1/23:0) and Sphingomyelin(d17:1/24:0).

The biomarker SM28 is preferably Sphingomyelin(d18:1/24:0).

The biomarker SM29 is preferably Sphingomyelin(d18:2/17:0).

The biomarker SM3 is preferably Sphingomyelin(d17:1/16:0).

The biomarker SM5 preferably refers to Sphingomyelin(d18:1/16:0) or more preferably to Sphingomyelin(d18:1/16:0) and Sphingomyelin(d16:1/18:0). Accordingly, the biomarker SM5 is Sphingomyelin(d18:1/16:0), ora combination of Sphingomyelin(d18:1/16:0) and Sphingomyelin(d16:1/18:0).

The amount of SM5 is, thus, preferably determined by determining the amount of Sphingomyelin(d18:1/16:0), or by determining the combined (and thus the total) amount of Sphingomyelin(d18:1/16:0) and Sphingomyelin(d16:1/18:0). Accordingly, the amount of SM5 is the amount of Sphingomyelin(d18:1/16:0), or the sum of the amounts of Sphingomyelin(d18:1/16:0) and Sphingomyelin(d16:1/18:0).

The biomarker SM8 is preferably Sphingomyelin(d18:2/18:1).

The biomarker SM9 preferably refers to Sphingomyelin(d18:1/18:1) or more preferably to Sphingomyelin(d18:1/18:1) and Sphingomyelin(d18:2/18:0). Accordingly, the biomarker SM9 is Sphingomyelin(d18:1/18:1), or a combination of Sphingomyelin(d18:1/18:1) and Sphingomyelin(d18:2/18:0).

The amount of SM9 is, thus, preferably determined by determining the amount of Sphingomyelin(d18:1/18:1), or by determining the combined (and thus the total) amount of Sphingomyelin(d18:1/18:1) and Sphingomyelin(d18:2/18:0). Accordingly, the amount of SM9 is the amount of Sphingomyelin(d18:1/18:1), or the sum of the amounts of Sphingomyelin(d18:1/18:1) and Sphingomyelin(d18:2/18:0).

As set forth above, the present invention is, inter alia, based on the determination of amount of at least three lipid metabolite biomarkers. In this context, it is noted that SM10, SM18, SM2, SM21, SM23, SM24, SM5, and SM9, and PC8 are considered as single biomarkers regardless whether the determination of these biomarkers is based on the determination of a single, two (or three) species. Thus, at least two further biomarkers have to be determined.

The sphingomyelin SM(dx¹:y¹/x²:y²) is preferably denoted to mean that the sphingomyelin comprises the “sphingosine-backbone” dx¹:y¹, wherein x¹ denotes the number of carbon atoms and y¹ the number of double bonds, and a “fatty acid amide” residue x²:y², wherein x² denotes the number of carbon atoms and y² the number of double bonds thereof.

The Sphingomyelin(d18:2/18:0) is preferably denoted to mean that the sphingomyelin comprises the “sphingosine-backbone” d18:2, which comprises 18 carbon atoms and 2 double bonds, and a “fatty acid amide” residue 18:0, which comprises 18 carbon atoms and 0 double bonds.

Cholesterylester (CE) Biomarkers

CE is the abbreviation for Cholesterylester.

In accordance with the present invention, the determination of two different cholesterylesters is contemplated, i.e. of cholesterylester C18:0 and/or cholesterylester C18:2. The biomarkers are well known in the art. The cholesterylester (Cx¹:y¹) is preferably denoted to mean that the cholesterylester comprises a fatty acid ester residue, wherein said fatty acid ester residue is Cx¹:y¹ which means that this residue comprises x¹ carbon atoms and y¹ double bonds.

For example, cholesterylester C18:0 is denoted to mean that the cholesterylester C18:0 comprises a fatty acid ester residue, wherein said fatty acid ester residue is C18:0 which means that this residue comprises 18 carbon atoms and 0 double bonds.

Phosphatidylcholine (PC) Biomarkers

PC is the abbreviation for Phosphatidylcholine. The Phosphatidylcholine PC (Cx¹:y¹ Cx²y²) is preferably denoted to mean that the phosphatidycholine comprises two fatty acid ester residues, wherein one fatty acid ester residue is Cx¹:y¹ which means that this residue comprises x¹ carbon atoms and y¹ double bonds, wherein one fatty acid ester residue is Cx²:y², which means that this residue comprises x² carbon atoms and y² double bonds.

For example, phosphatidylcholine (C16:0 C18:2) is preferably denoted to mean that the phosphatidycholine comprises two fatty acid ester residues, wherein one fatty acid ester residue is C16:0 which means that this residue comprises 16 carbon atoms and 0 double bonds, wherein one fatty acid ester residue is C18:2, which means that this residue comprises 18 carbon atoms and 2 double bonds.

The biomarker PC4 is Phosphatidylcholine (C16:0 C18:2). The biomarker PC8 preferably refers to Phosphatidylcholine(C18:0 C18:2) or more preferably to Phosphatidylcholine(C18:0 C18:2) and Phosphatidylcholine(C18:1 C18:1). Accordingly, the biomarker PC8 is Phosphatidylcholine(C18:0 C18:2), or a combination of Phosphatidylcholine(C18:0 C18:2) and Phosphatidylcholine(C18:1 C18:1).

The amount of PC8 is, thus, preferably determined by determining the amount of Phosphatidylcholine (C18:0 C18:2), or by determining the combined (and thus the total) amount of Phosphatidylcholine(C18:0 C18:2) and Phosphatidylcholine(C18:1 C18:1). Accordingly, the amount of PC8 is the amount of Phosphatidylcholine(C18:0 018:2), or the sum of the amounts of Phosphatidylcholine(C18:0 018:2) and Phosphatidylcholine(C18:1 C18:1).

Preferred Lipid Metabolite Biomarker Combinations

As set forth above, the method of the present invention for diagnosing a cardiac disease comprises the determination of the amounts of at least three lipid metabolite biomarkers (in addition to the at least one cardiac biomarker) in a sample of a subject. Thus, the determination of the amounts of a combination of at least three lipid metabolite biomarkers is contemplated. Preferred combinations of at least three lipid metabolite biomarkers to be determined in accordance with the present invention are described herein below. Moreover, preferred combinations of at least three markers are described in under “Embodiments/Items of the present invention”. The at least three lipid metabolite biomarkers as described under “Embodiments/Items of the present invention” can be used for the diagnosis of a cardiac disease as well.

The at least three lipid metabolite biomarkers to be used (in combination with at least one cardiac biomarker) for the diagnosis of cardiac disease are preferably:
i. at least one triacylglyceride, at least one cholesterylester, and at least one phosphatidylcholine;
ii. at least one triacylglyceride, at least one phosphatidylcholine, and at least one sphingomyelin;
iii. at least one triacylglyceride, at least one cholesterylester, and at least one sphingomyelin;
iv. at least one phosphatidylcholine, at least one cholesterylester, and at least one sphingomyelin;
v. Cholesterylester C18:2, SSS and Cer(d17:1/24:0);
vi. at least two sphingomyelins selected from the group consisting of SM2, SM3, SM5, SM18, SM23, SM24, and SM28, and at least one triacylglyceride selected from the group consisting of SOP2, SPP1 or PPO1 or selected from the group consisting of SOP2, SPP1 or PPP;
vii. at least two triacylglycerides selected from the group consisting of OSS2, SOP2, SPP1 and SSP2, and at least one sphingomyelin selected from the group consisting of SM23 and SM24;
viii. SM18, SM24 and SM28; or
ix. the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, or 206 of Table 2.

Preferred cholesterylester, triacylglyceride, phosphatidylcholine and sphingomyelin biomarkers to be determined and further preferred combinations of at least three lipid metabolite biomarkers are described below (in particular for items i., ii., iii., and iv.).

Preferably, the at least one triacylglyceride biomarker in i., ii., and iii. is selected from the group consisting of SOP2, OSS2, SPP1, SSP2, PPO1 and PPP, the at least one cholesterylester biomarker in i., iii. and iv. is selected from the group consisting of cholesterylester C18:2 and cholesterylester C18:0, the at least one phosphatidylcholine biomarker in i., ii., and iv. is selected from the group consisting of PC4 and PC8, and the at least one sphingomyelin biomarker in ii., iii. and iv. is selected from the group consisting of SM18, SM24, SM23, SM21, SM28, SM5, SM3, SM29 and SM8.

Alternatively, the least one triacylglyceride biomarker in i., ii., and iii. is selected from the group consisting of SOP2, OSS2, SPP1, SSP2, PPO1 and SPP1.

In a preferred embodiment, at least the amounts of the lipid metabolite biomarkers of items i., ii., iii., vi, vii. or ix. as set forth above are determined. In particular, the at least one triacylglyceride biomarker in i., ii. and iii. is selected from the group consisting of SOP2, OSS2, SSP2, PPO1 and PPP, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is selected from the group consisting of SM18, SM24, SM23, SM28, SM5, and SM3. Preferably, at least the amounts of the biomarkers of any panel of Table 2 which comprises these biomarkers are determined. More preferably, at least the amounts of the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 in Table 2 are determined. The combinations of lipid metabolite biomarkers as referred to in this paragraph are, preferably, determined if the HF (heart failure) to be diagnosed is heart failure with reduced left ventricular ejection fraction (HFrEF). The subject might show no symptoms of heart failure. Moreover, it is envisaged the HFrEF is selected from DCMP and ICMP.

The term “at least the amounts”, preferably means that, in principle, further biomarkers could be determined. In an embodiment, the term means “the amounts”. Preferably, the amounts of the at least three lipid metabolite biomarkers as referred to in step a) of the method disclosed herein, are used for the comparison in step b).

In an even further preferred embodiment, at least the amounts of the lipid metabolite biomarkers of i., ii., iii., vi., or ix. are determined, wherein the at least one triacylglyceride biomarker in i., ii. and iii. is SOP2 and/or OSS2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is selected from the group consisting of SM18, SM24, SM23, and SM3. Preferably, at least the amounts of the biomarkers of any panel of Table 2 which comprises these biomarkers are determined. In particular, at least the amounts of the biomarkers of panel 1, 2, 3, 4, 5, 6, 7, 8, 9, 10, 11, 12, 13, 14, 15, 16, 17, or 18 in Table 2 are determined. The combinations of lipid metabolite biomarkers as referred to in this paragraph are, preferably, determined if the HF (heart failure) to be diagnosed is heart failure with reduced left ventricular ejection fraction (HFrEF).

In an even further preferred embodiment, at least the amounts of the lipid metabolite biomarkers of i., ii., iii., vi., or ix. are determined, wherein the at least one triacylglyceride biomarker in i., ii. and iii. is SOP2 and/or OSS2, and/or (in particular and) the at least one cholesterylester biomarker in i. and iii. is cholesterylester C18:2, and/or (in particular and) the at least one phosphatidylcholine biomarker in i. and ii. is PC4, and/or (in particular and) the at least one sphingomyelin biomarker in ii. and iii. is SM23. Preferably, at least the amounts of the biomarkers of any panel of Table 2 which comprises these biomarkers are determined. In particular, at least the amounts of the biomarkers of panel 1, 2, 3, or 4 in Table 2 are determined. Preferably, at least the amounts of OSS2, PC4 and SM23 are determined (panel 1), in particular in combination with the determination of the amount of NT-proBNP or BNP (see elsewhere herein). Alternatively, the biomarkers of panels 3, 13 and 60 may be determined. Also preferably, at least the amounts of OSS2, cholesterylester C18:2 and SM23 are determined (panel 2). Alternatively, the biomarkers of panels 20, 21, 22, 23, 32 or 60 may be determined. The combinations of lipid metabolite biomarkers as referred to in this paragraph are for example determined if the cardiac disease to be disagnosed is HF (heart failure) such as heart failure with reduced left ventricular ejection fraction (HFrEF).

In an even further preferred embodiment, at least the amounts of the lipid metabolite biomarkers of iii. are determined, wherein the at least one triacylglyceride biomarker is SOP2 and/or OSS2, and/or (in particular and) the at least one cholesterylester biomarker is cholesterylester C18:2, and/or (in particular and) the at least one sphingomyelin biomarker is SM23. In particular, at least the amounts of SOP2, OSS2, PC4, Cholesterylester C18:2, SM18, SM28, SM24, SSP2, and SM23 are determined (panel 3). The combinations of lipid metabolite biomarkers as referred to in this paragraph are, preferably, determined if the cardiac disease to be disagnosed is HF (heart failure) such as heart failure with reduced left ventricular ejection fraction (HFrEF).

As set forth in the previous paragraph, it is, in particular, envisaged to determine the amounts (or to determine at least the amounts of) the lipid metabolite biomarkers comprised by panel 3, and thus of SOP2, OSS2, PC4, Cholesterylester C18:2, SM18, SM28, SM24, SSP2, and SM23. In particular early stages of heart failure can be determined. Thus, the subject may show no symptoms of heart failure. The LVEF may be mildly reduced. Thus, the HFrEF may be heart failure with a left ventricular ejection fraction of lower than 50% but larger than 35%. The same, e.g., applies to panels 1 and 2. Thus, early stages of heart failure can be diagnosed by using the biomarkers of panel 2 and in particular of panel 1. The subject to be tested may show no symptoms of heart failure. HFrEF may be heart failure with a left ventricular ejection fraction of lower than 50% but larger than 35%.

Also preferably, the amounts (or at least the amounts) of at least three lipid metabolite biomarkers, of at least four biomarkers, more preferably of at least five or six biomarkers, even more preferably of at least seven biomarkers and most preferably of at least eight biomarkers of the biomarkers of panel 3 are determined in step a) of the method of the present invention.

If the amounts of the biomarkers of panel 3 (or of the lipid metabolitebiomarkers as set forth in the previous paragraph) are determined, the method preferably does not comprise the further determination of amount of NT-proBNP and/or BNP. In particular, the method may not comprise the further determination of the amount of BNP and/or NT-proBNP and the comparison of the amount of BNP and/or NT-proBNP to a reference. Thus, the diagnosis of heart failure is not based on the determination of NT-proBNP. Accordingly, the method is a non-BNP and non-NT-proBNP based method. The same, e.g., may also apply to panels 1 and 2.

Alternatively, the method may comprise the further determination of amount of NT-proBNP and/or BNP. In particular, the method may comprise the further determination of the amount of BNP or NT-proBNP and the comparison of the amount of BNP or NT-proBNP to a reference.

If the amounts of the biomarkers of panel 3 (of at least three, four, five, six, seven or eight lipid metabolite biomarkers of the biomarkers of panel 3) are determined, preferably a correction for confounders is not carried out. Alternatively, a correction for confounders may be carried out. The same may, e.g., apply to panels 1 and 2.

In a preferred embodiment, at least the amounts of the lipid metabolite biomarkers shown in i., ii., iii. or vii. are determined, wherein the at least one triacylglyceride biomarker in i., ii. and iii. s selected from the group consisting of SOP2, OSS2, and SSP2, and/or (in particular and) the at least one cholesterylester biomarker in i. and iii. is cholesterylester 018:2, and/or (in particular and) the at least one phosphatidylcholine biomarker in i. and ii. is PC4, and/or (in particular and) the at least one sphingomyelin biomarker in ii. and iii. is selected from the group consisting of SM24, SM23, SM28, and SM3. Preferably, at least the amounts of the biomarkers of any panel of Table 2 which comprises these biomarkers are determined. More preferably, at least the amounts of the biomarkers of panel 19, 20, 21, 22, 23, 24, 25, 26, 27, 28, 29, 30, 31, 32, 33, 34, 35, or 36 in Table 2 are determined. The combinations of lipid metabolite biomarkers as referred to in this paragraph are, preferably, determined if the HF is HFrEF, and in particular DCMP (dilated cardiomyopathy). The subject to be tested, preferably, shows no symptoms of HF. Alternatively, the subject shows symptoms of HF.

In an even further preferred embodiment, at least the amounts of the lipid metabolite biomarkers shown in ii., or iii. are determined, wherein the at least one triacylglyceride biomarker in ii. and iii. is SOP2 and/or OSS2, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. and iii. is SM23 and/or SM24. Preferably, at least the amounts of the biomarkers of any panel of Table 2 which comprises these biomarkers are determined. More preferably, at least the amounts of the biomarkers of panel 31, 32, 33, 34, 35, or 36 in Table 2 are determined. The combinations of lipid metabolite biomarkers as referred to in this paragraph are, preferably, determined if the HF is HFrEF, and in particular DCMP (dilated cardiomyopathy).

In a preferred embodiment, at least the amounts of the lipid metabolite biomarkers shown ii., iii. or vi. are determined, wherein the at least one triacylglyceride biomarker in ii. and iii. is SOP2 and/or OSS2, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. is selected from the group consisting of SM18, SM24, and SM23. Preferably, at least the amounts of the biomarkers of any panel of Table 2 which comprises these biomarkers are determined. More preferably, at least the amounts of the biomarkers of panel 37, 38, 39, 40, 41, 42, 43, 44, 45, 46, 47, 48, 49, 50, 51, 52, 53, 54, 55, or 56 in Table 2 are determined. The combinations of lipid metabolite biomarkers as referred to in this paragraph are, preferably, determined if the HF is HFrEF, and in particular ICMP (ischemic cardiomyopathy).

In a further preferred embodiment, at least the amounts of the lipid metabolite biomarkers shown in iii. are determined, wherein the at least one triacylglyceride biomarker is SOP2, and/or (in particular and) the at least one cholesterylester biomarker is cholesterylester C18:2, and/or (in particular and) the at least one sphingomyelin biomarker is SM18 and/or SM23. Preferably, at least the amounts of the biomarkers of any panel of Table 2 which comprises these biomarkers are determined. More preferably, at least the amounts of the biomarkers of panel 51, 52, 53, 54, 55, or 56 in Table 2 are determined. The combinations of lipid metabolite biomarkers as referred to in this paragraph are, preferably, determined if the HF is HFrEF, and in particular ICMP (ischemic cardiomyopathy).

In a preferred embodiment, at least the amounts of the biomarkers of ii., iii., vi., vii., or ix. are determined, and wherein the at least one triacylglyceride biomarker in ii. and iii. is selected from the group consisting of SOP2, OSS2, and PPO1, and/or (in particular and) the at least one cholesterylester biomarker in iii. is cholesterylester C18:2, and/or (in particular and) the at least one phosphatidylcholine biomarker in ii. is PC4, and/or (in particular and) the at least one sphingomyelin biomarker in ii. and iii. is selected from the group consisting of SM24 and SM23. Preferably, at least the amounts of the biomarkers of any panel of Table 2 which comprises these biomarkers are determined. More preferably, at least the amounts of the biomarkers of panel 7, 8, 9, 10, 11, 12, 19, 20, 21, 22, 23, 24, 37, 38, 39, 40, 41, or 42 in Table 2 are determined. The combinations of lipid metabolite biomarkers as referred to in this paragraph are, preferably, determined if the HF is HFrEF, in particular asymptomatic HFrEF. Thus the subject to be tested preferably does not show symptoms of heart failure. Accordingly, the combinations of biomarkers are, preferably, determined for the diagnosis of HFrEF in a subject who does not show symptoms of heart failure.

In a further preferred embodiment, wherein at least the amounts of the biomarkers of iii. or vi are determined, wherein the at least one triacylglyceride biomarker in iii. is SOP2, and/or (in particular and) the at least one cholesterylester biomarker in iii. is cholesterylester C18:2, and/or (in particular and) the at least one sphingomyelin biomarker in iii. is selected from the group consisting of SM24 and SM23. Preferably, at least the amounts of the biomarkers of any panel of Table 2 which comprises these biomarkers are determined. More preferably, at least the amounts of the biomarkers of panel 7, 8, 9, 10, 11, or 12 in Table 2 are determined. The combinations of lipid metabolite biomarkers as referred to in this paragraph are, preferably, determined if the HF is HFrEF, in particular asymptomatic HFrEF. Thus the subject to be tested preferably does not show symptoms of heart failure. Accordingly, the combinations of biomarkers are, preferably, determined for the diagnosis of HFrEF in a subject who does not show symptoms of heart failure.

In a preferred embodiment, at least the amounts of the biomarkers of iii. or vii. are determined, wherein the at least one triacylglyceride biomarker in iii. is selected from the group consisting of SOP2 and OSS2, and/or (in particular and) the at least one cholesterylester biomarker in iii. is cholesterylester C18:2, and/or (in particular and) the at least one sphingomyelin biomarker in iii. is SM23. Preferably, at least the amounts of the biomarkers of any panel of Table 2 which comprises these biomarkers are determined. More preferably, at least the amounts of the biomarkers of panel 19, 20, 21, 22, 23, or 24 in Table 2 are determined. The combinations of lipid metabolite biomarkers as referred to in this paragraph are, preferably, determined if the HF or HFrEF is DCMP, in particular asymptomatic DCMP. Thus the subject to be tested preferably does not show symptoms of heart failure. Accordingly, the combinations of biomarkers are, preferably, determined for the diagnosis of HFrEF, in particular of DCMP, in a subject who does not show symptoms of heart failure.

In a preferred embodiment, at least the amounts of the biomarkers of iii. or vi. are determined, and wherein the at least one triacylglyceride biomarker in iii. is SOP2, and/or (in particular and) the at least one cholesterylester biomarker in iii. is cholesterylester C18:2, and/or (in particular and) the at least one sphingomyelin biomarker in iii. is selected from the group consisting of SM24 and SM23. Preferably, at least the amounts of the biomarkers of any panel of Table 2 which comprises these biomarkers are determined. More preferably, at least the amounts of the biomarkers of panel 37, 38, 39, 40, 41, or 42 in Table 2 are determined. The combinations of lipid metabolite biomarkers as referred to in this paragraph are, preferably, determined if the HF or HFrEF is ICMP, in particular asymptomatic ICMP. Thus the subject to be tested preferably does not show symptoms of heart failure. Accordingly, the combinations of biomarkers are, preferably, determined for the diagnosis of HFrEF, in particular of ICMP, in a subject who does not show symptoms of heart failure.

In an embodiment, the heart failure to be diagnosed is HF with preserved ejection fraction (HFpEF). For the diagnosis of HFpEF, preferably, at least the amounts of the biomarkers of ii., vi. or vii. as set forth above are determined, and wherein the at least one triacylglyceride biomarker in ii. is selected from the group consisting of SOP2, SSP2, SPP1 and PPO1, and/or (in particular and) the at least one phosphatidylcholine biomarker in ii. is selected from the group consisting of PC4 and PC8, and/or (in particular and) the at least one sphingomyelin biomarker in ii. is selected from the group consisting of SM18, SM24, SM23, SM28, SM5, and SM3. Preferably, at least the amounts of the biomarkers of any panel of Table 2 which comprises these biomarkers are determined. More preferably at least the amounts of the biomarkers of panel 79, 80, 81, 82, 83, 84, 85, 86, 87, 88, 89, 90, 91, 92, 93, 94, 95, 96, 97, 98, 99, 100, 101, or 102 in Table 2 are determined. Even more preferably, at least the amounts of the biomarkers of ii. are determined, and wherein the at least one triacylglyceride biomarker is selected from the group consisting of SSP2, SPP1 and PPO1, and/or (in particular and) the at least one phosphatidylcholine biomarker is PC4, and/or (in particular and) the at least one sphingomyelin biomarker is selected from the group consisting of SM24, SM5, and SM3. In particular, wherein at least the amounts of the biomarkers of panel 79, 81, 83, 85, 87, 89, 91, 93, 95, 97, 99, or 101 in Table 2 are determined for the diagnosis of HFpEF. Most preferably, at least the amounts of the biomarkers of ii. of are determined, and wherein the at least one triacylglyceride biomarker is SSP2, and/or (in particular and) the at least one phosphatidylcholine biomarker is PC4, and/or (in particular and) the at least one sphingomyelin biomarker is selected from the group consisting of SM24 and SM5. Preferably, at least the amounts of the biomarkers of panel 95, 97, 99, or 101 in Table 2 are determined.

In particular, the HFpEF to be diagnosed is asymptomatic. Accordingly, the HFpEF is diagnosed in a subject who does not show symptoms of heart failure. Preferably, at least the amounts of the biomarkers of ii. or vii. (in particular of ii.) are determined in this case, wherein the at least one triacylglyceride biomarker in ii. is selected from the group consisting of SPP1 and SSP2, and/or (in particular and) the at least one phosphatidylcholine biomarker in ii. is PC4, and/or (in particular and) the at least one sphingomyelin biomarker in ii. is selected from the group consisting of SM5 and SM3. Preferably, at least the amounts of the biomarkers of any panel of Table 2 which comprises these biomarkers are determined. In particular, at least the amounts of the biomarkers of panel 79, 80, 81, 82, 83, 84, 85, or 86 in Table 2 are determined. More preferably, at least the amounts of the biomarkers of ii. are determined, wherein the at least one triacylglyceride biomarker is SPP1, and/or (in particular and) the at least one phosphatidylcholine biomarker is PC4, and/or (in particular and) the at least one sphingomyelin biomarker is SM5. In particular, at least the amounts of the biomarkers of panel 79, 81, 83, or 85 in Table 2 are determined, preferably for the diagnosis of HFpEF in a subject who does not show symptoms of heart failure.

In a preferred embodiment, at least the amounts of the biomarkers of viii. (Panel 199) are determined, where CHF is to be diagnosed and NT-proBNP is not included, in particular, at least the amounts of the biomarkers of panel 3, 5, 9, 11, 17, 35, 39, 47, 53, 55, 62, 70, 77, 99, or 199 in Table 2 are determined.

In one embodiment, the amount of NT-proBNP or BNP is determined in addition to the amount of the resulting combinations of the at least three lipid metabolite biomarkers and the at least one additional cardiac biomarker set forth above. Thus, the present invention contemplates the determination of at least three lipid metabolite biomarkers, at least one additional cardiac biomarker, and of NT-proBNP or BNP, in particular NT-proBNP.

In another embodiment, the amount of NT-proBNP or BNP is not determined in addition to the amount of the resulting combinations of the at least three lipid metabolite biomarkers and the at least one additional cardiac biomarker set forth above (see elsewhere herein).

As set forth above, it is envisaged to determine the amounts of the biomarkers of any one of the panels 1 to 206. The biomarkers of panels 1 to 206 are shown in table 2 (second column). Preferred panels are panel 1, panel 2, panel 3 and panel 4, in particular panel 1 and panel 2. Another preferred panel is panel 200.

In a preferred embodiment, the amounts of the biomarkers of panel 1, 2, 3 or 4 are determined in step a) of the method of the present invention. In another preferred embodiment the biomarkers of panel 200 are determined.

In an especially preferred embodiment, the biomarkers of panel 1 are determined.

In another especially preferred embodiment, the biomarkers of panel 2 are determined.

For example, if the amounts of the biomarkers of panel 1 are determined, the amounts of SM23, OSS2 and PC4 are determined (in addition to the cardiac biomarker). As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In an embodiment, SM23 is Sphingomyelin (dl 7:1/24:1). In another embodiment, SM23 is Sphingomyelin(d18:1/23:1).

If the biomarkers of panel 1 are determined, it is in particular envisaged to determine:
the amount of OSS2, the amount of Sphingomyelin(d18:1/23:1) and the amount of PC4,
the amount of OSS2, the amount of Sphingomyelin (d17:1/24:1) and the amount of PC4, or
the amount of OSS2, the combined amount of Sphingomyelin(d18:1/23:1), Sphingomyelin(d18:2/23:0), and Sphingomyelin(d17:1/24:1), and the amount of PC4

For example, if the amounts of the biomarkers of panel 2 or 4 are determined, the amounts of SM23, OSS2 and Cholesterylester C18:2 are determined. As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In particular, SM23 is Sphingomyelin (d17:1/24:1) or Sphingomyelin(d18:1/23:1).

If the biomarkers of panel 2 or 4 are determined, it is in particular envisaged to determine:
the amount of OSS2, the amount of Sphingomyelin(d18:1/23:1) and the amount of Cholesterylester C18:2,
the amount of OSS2, the amount of Sphingomyelin (d17:1/24:1) and the amount of Cholesterylester C18:2, or
the amount of OSS2, the combined amount of Sphingomyelin(d18:1/23:1), Sphingomyelin(d18:2/23:0), Sphingomyelin(d17:1/24:1) and the amount of Cholesterylester C18:2

For example, if the amounts of the biomarkers of panel 200 are determined, the amounts of Cholesterylester C18:2, SM23, OSS2 and PC4 are determined (in addition to the at least one cardiac biomarker). As set forth elsewhere herein, SM23 can be i) Sphingomyelin(d18:1/23:1), ii) Sphingomyelin(d18:2/23:0), iii) Sphingomyelin(d17:1/24:1), or iv) a combination of Sphingomyelin(d18:1/23:1), Sphingomyelin(d18:2/23:0) and Sphingomyelin(d17:1/24:1). In an embodiment, SM23 is Sphingomyelin (d17:1/24:1). In another embodiment, SM23 is Sphingomyelin(d18:1/23:1).

If the biomarkers of panel 200 are determined, it is in particular envisaged to determine:
the amount of Cholesterylester C18:2, the amount of OSS2, the amount of Sphingomyelin(d18:1/23:1) and the amount of PC4,
the amount of Cholesterylester C18:2, the amount of OSS2, the amount of Sphingomyelin (d17:1/24:1) and the amount of PC4, or
the amount of Cholesterylester C18:2, the amount of OSS2, the combined amount of Sphingomyelin(d18:1/23:1), Sphingomyelin(d18:2/23:0), and Sphingomyelin(d17:1/24:1), and the amount of PC4

In addition to panel 1, panel 200 further comprises Cholesterylester C18:2.

In addition to panels 2 and 4, panel 200 further comprises PC4.

As set forth elsewhere herein, it is further envisaged to determine the amount of NT-proBNP or BNP in addition to the amounts of the biomarkers of the aforementioned panels (1 to 206) and in addition to the cardiac biomarker. For example, NT-proBNP or BNP is determined in addition to the amounts of the at least three lipid metabolite biomarkers of panel 1 and the at least one additional cardiac biomarker. Moreover, in an embodiment, it is envisaged that no correction for confounders is carried out (e.g. for panel 1).

In an embodiment of the determination of the at least three lipid metabolite biomarkers (in combination with the at least one cardiac biomarker) the biomarkers of panels 1, 2 and 200 are used in combination with NT-proBNP. Preferably, the determination is carried out for the diagnosis of HFrEF, in particular for the diagnosis of HFrEFwith a left ventricular ejection fraction of lower than 50% but larger than 35%. Thus, the subject to be tested preferably shall have a left ventricular ejection fraction of lower than 50% but larger than 35%. Preferably, the subject is asymptomatic and, thus, does not show symptoms of heart failure. Accordingly, asymptomatic heart failure is diagnosed.

In a preferred embodiment, the amounts of the biomarkers of panel 1, the amounts of HDLCholesterol and LDL-Cholesterol, and optionally the amount of NT-proBNP or BNP, in particular NT-proBNP are determined.

In a further preferred embodiment, the amounts of the biomarkers of panel 1, the amount of LDL-Cholesterol, and optionally the amount of NT-proBNP or BNP, in particular NT-proBNP are determined.

In a particular preferred embodiment, the amounts of the biomarkers of panel 1, the amount of HDL-Cholesterol, and optionally the amount of NT-proBNP or BNP, in particular NT-proBNP are determined.

Further preferred combinations of at least three lipid metabolite biomarkers for the diagnosis of a cardiac disease (which can be determined in addition to the least one cardiac marker) are disclosed in the following:

Moreover, the at least three lipid metabolite biomarkers, preferably are as follows. In particular it is envisaged to determine in step a. the amounts of
i. at least one sphingomyelin (SM) biomarker selected from the group consisting of SM18, SM21, SM23, SM24, SM28, SM3, SM5, SM2, SM9 and SM10, in particular at least one sphingomyelin (SM) biomarker selected from the group consisting of SM18, SM21, SM23, SM24, SM28, SM3 and SM5,
ii. at least one triacylglyceride biomarker selected from the group consisting of OSS2, PPO1, SOP2, SSP2 and SPP1, in particular at least one triacylglyceride biomarker selected from the group consisting of OSS2, PPO1, SOP2, and SSP2, and
iii. at least one further biomarker selected from the group consisting of Cer(d16:1/24:0), Cer(d18:1/24:1), Cholesterylester C18:2, PC4, PC8, Cer(d18:2/24:0), Cer(d17:1/24:0) and glutamic acid 1, in particular at least one further biomarker selected from the group consisting of Cer(d16:1/24:0), Cer(d18:1/24:1), Cholesterylester C18:2, and PC4.

Thus, at least one biomarker of i., at least one biomarker of ii., and at least one biomarker of iii. are determined. The same applies to the combinations below.

The aforementioned combinations (i.e. the determination of the aforementioned at least three lipid metabolite biomarkers) are preferably used for the diagnosis of a cardiac disease. In an embodiment, the cardiac disease is heart failure. In an embodiment, the heart failure is asymptomatic heart failure. Thus, the subject to be tested, preferably, does not show symptoms of heart failure. However, it is also envisaged that the subject may show symptoms of heart failure.

In a preferred embodiment, at least the amounts of i. at least one sphingomyelin biomarker selected from the group consisting of SM28, SM23, SM21, and SM5,
ii. SOP2 and/or OSS2, and
iii. Cholesterylester C18:2 and/or PC4 are determined.

The aforementioned combinations are preferably used for the diagnosis of a cardiac disease. In an embodiment, the cardiac disease is heart failure. In an embodiment, the heart failure is asymptomatic heart failure. However, it is also envisaged that the subject may show symptoms of heart failure.

In a further preferred embodiment, at least the amounts of
i. at least one sphingomyelin biomarker selected from the group consisting of SM28, SM21, and SM5, in particular SM28, or at least one sphingomyelin biomarker selected from the group consisting of SM18, SM21, and SM5, in particular SM18, and
ii. SOP2, and
iii. PC4

are determined.

The aforementioned combinations are preferably used for the diagnosis of a cardiac disease. In an embodiment, the cardiac disease is heart failure. In an embodiment, the heart failure is asymptomatic heart failure.

In a further preferred embodiment, wherein at least the amounts of
i. at least one sphingomyelin biomarker selected from the group consisting of SM18, SM2, SM23, SM24, SM28, SM3, and SM5,
ii. at least one triacylglyceride biomarker selected from the group consisting of OSS2, PPO1, SOP2, SSP2 and SPP1, and
iii. at least one further biomarker selected from the group consisting of Cer(d16:1/24:0), Cholesterylester C18:2, PC4 and glutamic acid 1 are determined.

The aforementioned combinations are preferably used for the diagnosis of a cardiac disease. In an embodiment, the cardiac disease is heart failure. In an embodiment, the heart failure is asymptomatic heart failure. Accordingly, the subject preferably does not show symptoms of heart failure.

In a further preferred embodiment, at least the amounts of
i. at least one sphingomyelin biomarker selected from the group consisting of SM23, SM2, and SM24,
ii. at least one triacylglyceride biomarker selected from the group consisting of SOP2, OSS2, and PPO1, and
iii. Cholesterylester C18:2 and/or PC4, in particular Cholesterylester C18:2,

are determined.

The aforementioned combinations are preferably used for the diagnosis of a cardiac disease. In an embodiment, the cardiac disease is heart failure. In an embodiment, the heart failure is asymptomatic heart failure. Accordingly, the subject preferably does not show symptoms of heart failure.

In particular, at least the amounts of SM23 and/or SM2, in particular of SM23, of SOP2, and of PC4 are determined. The aforementioned combinations are preferably used for the diagnosis of a cardiac disease. In an embodiment, the cardiac disease is heart failure. In an embodiment, the heart failure is asymptomatic heart failure. Accordingly, the subject to be tested preferably does not show symptoms of heart failure.

In a preferred embodiment of the present invention, at least the amounts of
i. at least one sphingomyelin biomarker selected from the group consisting of SM10, SM18, SM21, SM23, SM24, SM28, SM3 and SM9,
ii. at least one triacylglyceride biomarker selected from the group consisting of OSS2, PPO1, SOP2, SSP2 and SPP1, and
iii. at least one further biomarker selected from the group consisting of Cer(d16:1/24:0), Cer(d18:1/24:1), Cer(d17:1/24:0), Cer (d18:2/24:0), Cholesterylester C18:2, PC4 and PC8

are determined.

The aforementioned combinations are preferably used for the diagnosis of a cardiac disease. In an embodiment, the cardiac disease is heart failure. In an embodiment, the heart failure is symptomatic heart failure. Accordingly, the subject to be tested preferably shows symptoms of heart failure.

In further preferred embodiment
i. at least one sphingomyelin biomarker is selected from the group consisting of SM18, SM3, SM24, and SM23, in particular SM18,
ii. SOP2 and/or OSS2, in particular SOP2, and
iii. Cholesterylester C18:2 and/or PC4, in particular PC4,

are determined.

In particular, least the amounts of SM18 and/or SM24, SOP2, and PC4 are determined.

The aforementioned combinations are preferably used for the diagnosis of a cardiac disease. In an embodiment, the cardiac disease is heart failure. In an embodiment, the heart failure is symptomatic heart failure. Accordingly, the subject to be tested preferably shows symptoms of heart failure.

In a preferred embodiment, wherein at least the amounts of
i. at least one sphingomyelin biomarker selected from the group consisting of SM23, SM18, SM24, SM28, SM2 and SM3, in particular at least one sphingomyelin biomarker selected from the group consisting of SM23, SM18, SM24, and SM28,
ii. OSS2 and/or SOP2, in particular OSS2,
iii. Cholesterylester C18:2 and/or PC4

are determined.

In particular, at least the amounts of SM23, OSS2, and Cholesterylester C18:2 are determined.

The aforementioned combinations are preferably used for the diagnosis of heart failure, in particular of HFrEF. Preferably, the subject does not show symptoms of heart failure. Also preferably, the subject shows symptoms of heart failure.

In a further preferred embodiment, at least the amounts of
i. at least one sphingomyelin biomarker selected from the group consisting of SM23 SM18, SM2, SM24 and SM28,
ii. SOP2, and
iii. Cholesterylester C18:2

are determined.

In particular, at least the amounts of biomarkers SM23, SOP2, and Cholesterylester C18:2 are determined.

The aforementioned combinations are preferably used for the diagnosis of heart failure, in particular of HFrEF. In an embodiment, the heart failure is asymptomatic heart failure, in particular asymptomatic HFrEF. Accordingly, the subject to be tested preferably does not show symptoms of heart failure.

In a preferred embodiment, wherein at least the amounts of
i. at least one sphingomyelin biomarker selected from the group consisting of SM18, SM23, SM3 or at least one sphingomyelin biomarker selected from the group consisting of SM28, SM23 and SM3,
ii. OSS2, and
iii. Cholesterylester C18:2 and/or PC4

are determined.

In particular, at least the amounts of SM18 and/or SM28, of OSS2, and of Cholesterylester C18:2 are determined.

The aforementioned combinations are preferably used for the diagnosis of heart failure, in particular of HFrEF. In an embodiment, the heart failure is symptomatic heart failure, in particular symptomatic HFrEF. Accordingly, the subject to be tested preferably shows symptoms of heart failure.

In a preferred embodiment of the present invention, at least the amounts of
i. at least one sphingomyelin biomarker selected from the group consisting of SM23, SM24, SM28, and SM3, in particular at least one sphingomyelin biomarker selected from the group consisting of SM23, SM24, and SM28,
ii. SOP2 and/or OSS2, and
iii. Cholesterylester C18:2 and/or PC4

are determined.

In particular, at least the amounts of SM23, of SOP2, and of Cholesterylester C18:2 are determined.

The aforementioned combinations are preferably used for the diagnosis of heart failure, more preferably of HFrEF, and most preferably of DCMP. Preferably, the subject does not show symptoms of heart failure. Also preferably, the subject shows symptoms of heart failure.

In a further preferred embodiment of the present invention, at least the amounts of
i. SM23,
ii. OSS2 and/or SOP2, and
iii. Cholesterylester C18:2

are determined.

In particular, at least the amounts of SM23, OSS2, and Cholesterylester 018:2 are determined.

The aforementioned combinations are preferably used for the diagnosis of heart failure, more preferably of HFrEF, and most preferably of DCMP, in particular of asymptomatic HFrEF and/or DCMP. Thus, the subject to be tested preferably does not show symptoms of HF.

In a preferred embodiment of the present invention at least the amounts of
i. SM28 and/or SM3,
ii. SOP2 and/or OSS2, and
iii. PC4

are determined.

In particular, at least the amounts of SM28, SOP2, and of PC4 are determined.

The aforementioned combinations are preferably used for the diagnosis of heart failure, more preferably of HFrEF, and most preferably of DCMP, in particular of symptomatic HFrEF and/or DCMP. Thus, the subject to be tested preferably shows symptoms of HF.

In another preferred embodiment, wherein at least the amounts of
i. at least one sphingomyelin biomarker selected from the group consisting of SM23, SM24, SM28 and SM18, in particular at least one sphingomyelin biomarker selected from the group consisting of SM23, SM24, and SM28,
ii. SOP2, and
iii. Cholesterylester C18:2

are determined.

In particular, at least the amounts of SM23, SOP2, and Cholesterylester C18:2 are determined.

The aforementioned combinations are preferably used for the diagnosis of heart failure, more preferably of HFrEF, and most preferably of ICMP. In one (preferred) embodiment, the subject does not show symptoms of heart failure. In another embodiment, the subject shows symptoms of heart failure.

In a further preferred embodiment, at least the amounts of SM18, SOP2, and Cholesterylester C18:2 are determined. The aforementioned combination is preferably used for the diagnosis of heart failure, more preferably of HFrEF, and most preferably of ICMP, in particular of symptomatic HFrEF and/or ICMP. Thus, the subject to be tested preferably shows symptoms of HF.

For the diagnosis of asymptomatic heart failure it is further envisaged that the at least three are biomarkers are:
i. at least one sphingomyelin biomarker selected from the group consisting of SM23, SM2, and SM24,
ii. at least one triacylglyceride biomarker selected from the group consisting of SOP2, OSS2, and PPO1, and
iii. at least one further biomarker selected from Cholesterylester C18:2, PC4, and SM5.

Thus, the subject preferably does not show symptoms of heart failure.

For the diagnosis of asymptomatic ICMP it is further envisaged that the at least three lipid metabolite biomarkers are markers are selected from the group consisting of SM24, SM5, SM23, SOP2 and PPO1. Preferably, the amounts of SM24, SM5 and SOP2, or of SM23, SM5 and PPO1 are determined.

In one embodiment, the amount of NT-proBNP or BNP is determined in addition to the amount of the resulting combinations of the at least three lipid metabolite biomarkers set forth above. In another embodiment, the amount of NT-proBNP or BNP is not determined in addition to the amount of the resulting combinations of the at least three lipid metabolite biomarkers set forth above (see elsewhere herein).

Additional Preferred Combinations of at Least Three Lipid Metabolite Biomarkers for the Diagnosis of a Cardiac Disease Such as Heart Failure

In a preferred embodiment, the least three lipid metabolite biomarkers are:
i. at least one sphingomyelin (SM) biomarker selected from the group consisting of SM18, SM23, SM24, SM3, SM5, SM2, and SM28, and in particular at least one sphingomyelin (SM) biomarker selected from the group consisting of SM18, SM23, SM24, SM3, and SM5,
ii. at least one triacylglyceride biomarker selected from the group consisting of OSS2, SOP2, SSP2 and PPO1, in particular at least one triacylglyceride biomarker selected from the group consisting of OSS2 and SOP2, and
iii. at least one further biomarker selected from the group consisting of Cholesterylester C18:2 and PC4.

Thus, at least one biomarker of i., at least one biomarker of ii., and at least one biomarker of iii. are determined. The same applies to the combinations below.

In a preferred embodiment, at least the amounts of
i. at least one sphingomyelin biomarker selected from the group consisting of SM18, SM23, SM24, SM3, and SM5,
ii. at least one triacylglyceride biomarker selected from the group consisting of OSS2 and SOP2, and
iii. at least one further biomarker selected from the group consisting of Cholesterylester C18:2 and PC4 are determined.

In particular, at the least the amounts of
i. at least one sphingomyelin biomarker selected from the group consisting of SM18, SM3, and SM5, in particular SM18, and
ii. at least one triacylglyceride biomarker selected from the group consisting of OSS2 and SOP2, in particular SOP2, and
iii. at least one further biomarker selected from the group consisting of Cholesterylester C18:2 and PC4, in particular PC4

are determined.

The aforementioned combinations (i.e. the determination of the aforementioned at least three lipid metabolite biomarkers) are preferably used for the diagnosis of a cardiac disease. In an embodiment, the cardiac disease is heart failure. In an embodiment, the heart failure is asymptomatic heart failure. Thus, the subject to be tested, preferably, does not show symptoms of heart failure. However, it is also envisaged that the subject may show symptoms of heart failure.

In a preferred embodiment, at least the amounts of
i. at least one sphingomyelin biomarker selected from the group consisting of SM18, SM2, SM23, SM24, SM28, and SM5,
ii. at least one triacylglyceride biomarker selected from the group consisting of OSS2, PPO1, SOP2, and SSP2, and
iii. at least one further biomarker selected from the group consisting of Cholesterylester C18:2 and PC4

are determined.

In a more preferred embodiment, at least the amounts of
i. at least one sphingomyelin biomarker selected from the group consisting of SM23, SM24, SM18, and SM2, in particular SM23,
ii. at least one triacylglyceride biomarker selected from the group consisting of SOP2, OSS2, and PPO1, in particular SOP2, and
iii. at least one further biomarker selected from the group consisting of Cholesterylester C18:2 and PC4, in particular Cholesterylester C18:2, are determined.

In an even more preferred embodiment, at least the amounts of
i. at least one sphingomyelin biomarker selected from the group consisting of SM23, SM18, and SM2, in particular SM23,
ii. SOP2, and
iii. Cholesterylester C18:2 and/or PC4, in particular Cholesterylester C18:2, are determined.

The aforementioned combinations are preferably used for the diagnosis of a cardiac disease. In an embodiment, the cardiac disease is heart failure. In an embodiment, the heart failure is asymptomatic heart failure. Accordingly, the subject preferably does not show symptoms of heart failure.

In a preferred embodiment,
i. at least one sphingomyelin biomarker selected from the group consisting of SM18, SM23, SM24, SM28, and SM3,
ii. at least one triacylglyceride biomarker selected from the group consisting of OSS2 and SOP2, and
iii. at least one further biomarker selected from the group consisting of Cholesterylester C18:2 and PC4 are determined.

Preferably, at least the amounts of
i. SM18,
ii. SOP2, and
iii. Cholesterylester 018:2,

are determined.

In particular, at least the amounts of SM18 and/or SM24, in particular SM18, of SOP2, and of PC4 are determined.

The aforementioned combinations are preferably used for the diagnosis of a cardiac disease. In an embodiment, the cardiac disease is heart failure. In an embodiment, the heart failure is symptomatic heart failure. Accordingly, the subject to be tested preferably shows symptoms of heart failure.

In a further preferred embodiment, at least the amounts of
i. at least one sphingomyelin biomarker selected from the group consisting of SM23, SM18, SM3, SM24, SM 28 in particular at least one sphingomyelin biomarker selected from the group consisting of SM23 and SM18,
ii. at least one triacylglyceride biomarker selected from the group consisting of OSS2, SOP2 and PPO1, in particular OSS2, and
iii. at least one further biomarker selected from the group consisting of Cholesterylester C18:2 and PC4

are determined.

In particular, at least the amounts of SM23, OSS2, and Cholesterylester C18:2 are determined.

The aforementioned combinations are preferably used for the diagnosis of heart failure, in particular of HFrEF. Preferably, the subject does not show symptoms of heart failure. Also preferably, the subject shows symptoms of heart failure.

In a further preferred embodiment, at least the amounts of
i. at least one sphingomyelin biomarker selected from the group consisting of SM23 and SM24, in particular SM23,
ii. at least one triacylglyceride biomarker selected from the group consisting of SOP2, and OSS2, in particular SOP2, and
iii. at least one further biomarker selected from the group consisting of Cholesterylester C18:2 and PC4, in particular Cholesterylester C18:2

are determined.

In particular, at least the amounts of SM23, SOP2, and Cholesterylester C18:2 are determined.

The aforementioned combinations are preferably used for the diagnosis of heart failure, in particular of HFrEF. In an embodiment, the heart failure is asymptomatic heart failure, in particular asymptomatic HFrEF. Accordingly, the subject to be tested preferably does not show symptoms of heart failure.

In a further preferred embodiment, at least the amounts of
i. at least one sphingomyelin biomarker selected from the group consisting of SM18, SM23, SM3, SM28 and SM24, in particular at least one sphingomyelin biomarker selected from the group consisting of SM18, SM23 and SM3,
ii. at least one triacylglyceride biomarker selected from the group consisting of SOP2 and OSS2, and
iii. at least one further biomarker selected from the group consisting of Cholesterylester C18:2 and PC4

are determined.

In particular, at least the amounts of SM18, of OSS2, and of Cholesterylester C18:2 are determined.

The aforementioned combinations are preferably used for the diagnosis of heart failure, in particular of HFrEF. In an embodiment, the heart failure is symptomatic heart failure, in particular symptomatic HFrEF. Accordingly, the subject to be tested preferably shows symptoms of heart failure.

In a further preferred embodiment, at least the amounts of
i. at least one sphingomyelin biomarker selected from the group consisting of SM23, SM24, SM3 and SM28, in particular at least one sphingomyelin biomarker selected from the group consisting of SM23 and SM24,
ii. at least one triacylglyceride biomarker selected from the group consisting of SOP2, and OSS2, and
iii. at least one further biomarker selected from the group consisting of Cholesterylester C18:2 and PC4

are determined.

In particular, at least the amounts of SM23, of SOP2, and of Cholesterylester C18:2 are determined.

The aforementioned combinations are preferably used for the diagnosis of heart failure, more preferably of HFrEF, and most preferably of DCMP. Preferably, the subject does not show symptoms of heart failure. Also preferably, the subject shows symptoms of heart failure.

In a further preferred embodiment, wherein at least the amounts of
i. at least one sphingomyelin biomarker selected from the group consisting of SM23 and SM24,
ii. at least one triacylglyceride biomarker selected from the group consisting of SOP2 and OSS2, and
iii. Cholesterylester C18:2

are determined.

In particular, at least the amounts of SM23, OSS2, and Cholesterylester 018:2 are determined.

The aforementioned combinations are preferably used for the diagnosis of heart failure, more preferably of HFrEF, and most preferably of DCMP, in particular of asymptomatic HFrEF and/or DCMP. Thus, the subject to be tested preferably does not show symptoms of HF.

In a further preferred embodiment, at least the amounts of
i. at least one sphingomyelin biomarker selected from the group consisting of SM3, SM24 and SM28,
ii. at least one triacylglyceride biomarker selected from the group consisting of SOP2 and OSS2, and
iii. at least one further biomarker selected from the group consisting of Cholesterylester

C18:2 and PC4 are determined.

In particular, at least the amounts of SM3, OSS2, and of PC4 are determined.

The aforementioned combinations are preferably used for the diagnosis of heart failure, more preferably of HFrEF, and most preferably of DCMP, in particular of symptomatic HFrEF and/or DCMP. Thus, the subject to be tested preferably shows symptoms of HF.

In a further preferred embodiment, at least the amounts of
i. at least one sphingomyelin biomarker selected from the group consisting of SM18, SM23, and SM24, in particular at least one sphingomyelin biomarker selected from the group consisting of SM18 and SM23,
ii. at least one triacylglyceride biomarker selected from the group consisting of SOP2 and PPO1, in particular SOP2, and
iii. at least one further biomarker selected from the group consisting of Cholesterylester C18:2 and PC4, in particular Cholesterylester C18:2

are determined.

In particular, at least the amounts of SM18, SOP2, and Cholesterylester C18:2 are determined.

The aforementioned combinations are preferably used for the diagnosis of heart failure, more preferably of HFrEF, and most preferably of ICMP. Preferably, the subject does not show symptoms of heart failure. Also preferably, the subject shows symptoms of heart failure.

In a further preferred embodiment, at least the amounts of
i. at least one sphingomyelin biomarker selected from the group consisting of SM24 and SM23,
ii. at least one triacylglyceride biomarker selected from the group consisting of SOP2 and PPO1, and
iii. at least one further biomarker selected from the group consisting of Cholesterylester C18:2 and PC4, in particular Cholesterylester C18:2

are determined.

In particular, at least the amounts of SM24, SOP2, and Cholesterylester C18:2 are determined.

The aforementioned combinations are preferably used for the diagnosis of heart failure, more preferably of HFrEF, and most preferably of ICMP, in particular of asymptomatic HFrEF and/or ICMP. Thus, the subject to be tested preferably does not show symptoms of HF.

In an even further preferred embodiment, at least the amounts of SM18, SOP2, and Cholesterylester C18:2 are determined. The aforementioned combination is preferably used for the diagnosis of heart failure, more preferably of HFrEF, and most preferably of ICMP, in particular of symptomatic HFrEF and/or ICMP. Thus, the subject to be tested preferably shows symptoms of HF.

Moreover, for the diagnosis of asymptomatic heart failure it is envisaged to determine the amounts of
i. at least one sphingomyelin biomarker selected from the group consisting of SM23, SM24, SM18, and SM2, in particular SM23,
ii. at least one triacylglyceride biomarker selected from the group consisting of SOP2, OSS2, and PPO1, in particular SOP2, and
iii. at least one further biomarker selected from the group consisting of Cholesterylester C18:2, PC4, SM5 and SSP2, in particular Cholesterylester C18:2.

Moreover, for the diagnosis of asymptomatic heart failure it is envisaged to determine the amounts of
i. at least one sphingomyelin biomarker selected from the group consisting of SM23, SM18, and SM2, in particular SM23,
ii. SOP2, and
iii. at least one further biomarker selected from the group consisting of SM5, Cholesterylester C18:2 PC4, in particular SM5.

Moreover, for the diagnosis of asymptomatic HFrEF it is envisaged to determine the amounts of
i. at least one sphingomyelin biomarker selected from the group consisting of SM23 and SM24,
ii. at least one triacylglyceride biomarker selected from the group consisting of SOP2, and OSS2, in particular SOP2, and
iii. at least one further biomarker selected from the group consisting of Cholesterylester C18:2 and SM28, in particular Cholesterylester C18:2.

Moreover, for the diagnosis of asymptomatic DCMP it is envisaged to determine the amounts of
i. at least one sphingomyelin biomarker selected from the group consisting of SM23 and SM24, in particular SM23,
ii. at least one triacylglyceride biomarker selected from the group consisting of SOP2 and OSS2, in particular OSS2 and
iii. Cholesterylester C18:2 and SSP2, in particular Cholesterylester C18:2.

Moreover, for the diagnosis of asymptomatic ICMP it is envisaged to determine the amounts of
i. at least one sphingomyelin biomarker selected from the group consisting of SM24 and SM23, in particular SM24,
ii. at least one triacylglyceride biomarker selected from the group consisting of SOP2 and PPO1, in particular SOP2, and
iii. at least one further biomarker selected from the group consisting of SM5, Cholesterylester C18:2 and PC4, in particular Cholesterylester C18:2, in particular SM5, or in particular PC4.

In one embodiment, the amount of NT-proBNP or BNP is determined in addition to the amount of the resulting combinations of the at least three lipid metabolite biomarkers set forth above (and the additional cardiac biomarker). In another embodiment, the amount of NT-proBNP or BNP is not determined in addition to the amount of the resulting combinations of the at least three lipid metabolite biomarkers set forth above (see elsewhere herein).

If the lipid metabolite biomarker is a triacylglyceride it is preferably envisaged that the determination of the amount of this biomarker does not encompass the derivatization of this marker. Accordingly, it is envisaged that the determination of this biomarker is not based on the determination of one or more of the fatty acid residues derived from said triacylglyceride. Accordingly, the amount of the entire triacylglyceride is measured.

In an embodiment, the lipid metabolite biomarkers of panel 1 are determined in combination with at least one general lipid cardiac biomarker. In a preferred embodiment, the lipid metabolite biomarkers of panel 1 are determined in combination with at least one apolipoprotein biomarker. In another preferred embodiment, the lipid metabolite biomarkers of panel 1 are determined in combination with at least one lipoprotein subfraction biomarker. In an even further preferred embodiment, the lipid metabolite biomarkers of panel 1 are determined in combination with at least one inflammation biomarker. In addition, BNP or NT-proBNP can be determined.

In an embodiment, the lipid metabolite biomarkers of panel 2 are determined in combination with at least one general lipid cardiac biomarker. In a preferred embodiment, the lipid metabolite biomarkers of panel 2 are determined in combination with at least one apolipoprotein biomarker. In another preferred embodiment, the lipid metabolite biomarkers of panel 2 are determined in combination with at least one lipoprotein subfraction biomarker. In an even further preferred embodiment, the lipid metabolite biomarkers of panel 2 are determined in combination with at least one inflammation biomarker. In addition, BNP or NT-proBNP can be determined.

In an embodiment, the lipid metabolite biomarkers of panel 200 are determined in combination with at least one general lipid cardiac biomarker. In a preferred embodiment, the lipid metabolite biomarkers of panel 200 are determined in combination with at least one apolipoprotein biomarker. In another preferred embodiment, the lipid metabolite biomarkers of panel 200 are determined in combination with at least one lipoprotein subfraction biomarker. In an even further preferred embodiment, the lipid metabolite biomarkers of panel 200 are determined in combination with at least one inflammation biomarker. In addition, BNP or NT-proBNP can be determined.

The term “subject” as used herein relates to animals and, preferably, to mammals. More preferably, the subject is a primate and, most preferably, a human. The subject may be a female or, in particular, a male subject. The subject to be tested, preferably, is suspected to suffer from a cardiac disease. The subject to be tested may have a history of myocardial infarction and/or may suffer from diabetes type II. Preferably, the subject is an adult. More preferably, the subject is older than 40 years of age, and most preferably older than 50 years of age. Further, it is envisaged that the subject is older than 54 years or age, but younger than 61 years of age.

More preferably, the subject may already show symptoms of the cardiac disease such as heart failure. Most preferably, the subject does not show symptoms of the cardiac disease such as heart failure. Also encompassed as subjects are those, which belong into risk groups or subjects that are included in disease screening projects or measures.

In aged and overweight subjects, NT-proBNP and BNP are less reliable markers for heart failure. However, the determination of the at least three lipid metabolite biomarkers (in combination with the at least one additional cardiac biomarker) as referred herein may improve the diagnostic accuracy in aged or overweight subjects. Therefore, the subject may be aged or overweight:

A subject who shows symptoms of heart failure in particular shows symptoms according to NYHA class II and/or III. Accordingly, the subject who shows symptoms of heart failure, preferably, has a slight or marked limitation of physical activity, is comfortable at rest, but ordinary or less than ordinary physical activity results in undue breathlessness, fatigue, or palpitations (in this subject). A subject who does not show symptoms of heart failure preferably has no limitation of physical activity, and ordinary physical activity results in undue breathlessness, fatigue, or palpitations (in this subject).

In a preferred embodiment, the present invention envisages the diagnosis of early stages of heart failure. Thus, the term “heart failure” means, in an embodiment, “early stage of heart failure”. For example, the presence or, in particular, the absence of an early stage of heart failure can be diagnosed.

An early stage of heart failure may be, in an embodiment, heart failure according to NYHA class I. In another embodiment, the early stage of heart failure may be heart failure according to NYHA class II. Thus, heart failure according to NYHA class I or NYHA class II can be diagnosed by using the at least three lipid metabolite biomarkers as referred to herein (e.g. the biomarkers of panel 1) (in combination with the at least one additional cardiac biomarker).

With respect to HFrEF, an early stage of heart failure further may mean that the left ventricular ejection fraction is lower than 50% but larger than 35%. Thus, heart failure with a left ventricular ejection fraction that is lower than 50% but larger than 35% can be diagnosed by using the at least three lipid metabolite biomarkers (in combination with the at least one additional cardiac biomarker as referred to herein (e.g. the biomarkers of panel 1). In an embodiment, the subject preferably shows no symptoms of heart failure.

Preferred combinations of at least three lipid metabolite biomarkers that are used for diagnosing a cardiac disease (in combination with the at least one additional cardiac biomarker), if the subject shows symptoms of heart failure, are described in connection with the diagnosis of symptomatic heart failure.

Preferred combinations of at least three lipid metabolite biomarkers that are used for diagnosing a cardiac disease (in combination with the at least one additional cardiac biomarker, if the subject does not show symptoms of heart failure, are described in connection with the diagnosis of asymptomatic heart failure.

Preferably, the subject, however, is besides the aforementioned diseases and disorders apparently healthy. Also preferably, the subject shall not suffer from apoplex (stroke), myocardial infarction within the last 4 month before the sample has been taken or from acute or chronic inflammatory diseases and malignant tumors. In particular, the subject shall not suffer from stroke, myocardial infarction or unstable angina at the time at which the sample to be tested is obtained. Furthermore, the subject is preferably in stable medications within the last 4 weeks before the sample was taken.

In a preferred embodiment, the subject to be tested does not suffer from impaired renal function. However, it is also contemplated that the subject suffers from impaired renal function. Renal function can be assessed e.g. by determining the glomerular filtration rate (GFR). Preferably, a subject suffers from impaired renal function if the GFR is below 60 mL/min/1.73 m², or in particular below 50 mL/min/1.73 m². Preferably, a subject does not suffer from impaired renal function if the GFR is above 60 mL/min/1.73 m², or in particular above 70 mL/min/1.73 m².

More preferably, the subject who does not suffer impaired renal disease does not suffer from chronic kidney disease stages 3 to 5 (for the classification, see e.g. National Kidney Foundation, 2002. K/DOQI clinical practice guidelines for chronic kidney disease: evaluation, classification, and stratification. Am. J. Kidney Dis. 39, S1-266 which herewith is incorporated by reference in its entirety.)

GFR may be accurately calculated by comparative measurements of substances in the blood and urine, or estimated by formulas using just a blood test result (eGFR). Usually these estimates are used in clinical practice in particular in elderly and sick patients where reliable urine collections are difficult. These tests are important in assessing the excretory function of the kidneys, for example in grading of chronic renal insufficiency.

eGFR is associated with GFR via a large clinical study (National Kidney Foundation (February 2002). “K/DOQI clinical practice guidelines for chronic kidney disease: evaluation, classification, and stratification”. American Journal of Kidney Diseases 39 (2 Suppl 1): 51-266. doi:10.1016/50272-6386(02)70081-4. PMID 11904577). For clinical assessment scales of eGFR and GFR can be used interchangeably.

The term “sample” as used herein refers to samples from body fluids, preferably, blood, plasma, serum, saliva or urine, or samples derived, e.g., by biopsy, from cells, tissues or organs, in particular from the heart. More preferably, the sample is a blood, plasma or serum sample, most preferably, a plasma sample. Biological samples can be derived from a subject as specified elsewhere herein. Techniques for obtaining the aforementioned different types of biological samples are well known in the art. For example, blood samples may be obtained by blood taking while tissue or organ samples are to be obtained, e.g., by biopsy.

The aforementioned samples are, preferably, pre-treated before they are used for the method of the present invention. As described in more detail below, said pre-treatment may include treatments required to release or separate the compounds or to remove excessive material or waste. Suitable techniques comprise centrifugation, extraction, fractioning, ultrafiltration, protein precipitation followed by filtration and purification and/or enrichment of compounds. Moreover, other pre-treatments are carried out in order to provide the compounds in a form or concentration suitable for compound analysis. For example, if gas-chromatography coupled mass spectrometry is used in the method of the present invention, it will be required to derivatize the compounds prior to the said gas chromatography. Suitable and necessary pre-treatments depend on the means used for carrying out the method of the invention and are well known to the person skilled in the art. Pre-treated samples as described before are also comprised by the term “sample” as used in accordance with the present invention.

As set forth herein below in more detail, the determination of the amount of a biomarker as referred to herein, preferably includes a separation step, i.e. a step in which compounds comprised by the sample are separated. This applies e.g., if the amounts of lipid metabolite biomarkers are determined. Preferably, the separation of the compounds is carried by chromatography, in particular by liquid chromatography (LC) or high performance liquid chromatography (HPLC). Preferably, the pre-treatment of the sample should allow for a subsequent separation of compounds, in particular the metabolite biomarkers as referred to above, comprised by the sample. Molecules of interest, in particular the lipid metabolite biomarkers as referred to above may be extracted in an extraction step which comprises mixing of the sample with a suitable extraction solvent. The extraction solvent shall be capable of precipitating the proteins in a sample, thereby facilitating the, preferably, centrifugation-based, removal of protein contaminants which otherwise would interfere with the subsequent analysis of the biomarkers as referred above. Preferably, the at least three lipid metabolite biomarkers as referred to herein are soluble in the extraction solvent. More preferably, the extraction solvent is a one phase solvent. Even more preferably, the extraction solvent is a mixture comprising a first solvent selected from the group consisting of dichloromethane (DCM), chloroform, tertiary butyl methyl ether (tBME or MTBE, also known as 2-methoxy-2-methylpropane), ethyl ethanoate, and isooctane, and a second solvent selected from the group consisting of methanol, ethanol, isopropanol and dimethyl sulfoxide (DMSO). In an embodiment the extraction solvent comprises methanol and DCM, in particular a ratio of about 2:1 to about 3.2, preferably a ratio of about 2:1 or about 3:2. The term “about” as used herein refers to either the precise value indicated afterwards or to a value differing+/−20%, +/−10%, +/−5%, +/−2% or +/−1% from the said precise value.

Preferably, the pretreatment of the sample comprises an extraction step with a suitable extraction solvent. This extraction step additionally results in the precipitation of proteins comprised by the sample. Subsequently, the proteins comprised by the sample are removed by centrifugation.

As as set forth above, the method of the present invention may further comprise the determination of the amount of one or more protein markers useful in the diagnosis of a cardiac disease, in particular of the cardiac biomarkers Lp(a), CRP/hsCRP, LP PLA2, and apolipoprotein B (and in certain embodiments the protein marker NT-proBNP or BNP). These markers are preferably determined by using antibodies which specifically bind to the markers (or alternatively by other methods known in the art). The pretreatment as described in the two paragraphs above, thus, does not apply to the determination of protein markers. Preferably, the pre-treatment applies to the determination of the amount of the lipid metabolite biomarkers, i.e. of the specific sphingomyelins, specific triacylglycerides, specific cholesterylesters, specific phosphatidylcholines, specific ceramides and/or glutamic acid.

The term “determining the amount”, in particular of the lipid metabolite biomarkers, as used herein refers to determining at least one characteristic feature of a biomarker to be determined by the method of the present invention in the sample. Characteristic features in accordance with the present invention are features which characterize the physical and/or chemical properties including biochemical properties of a biomarker. Such properties include, e.g., molecular weight, viscosity, density, electrical charge, spin, optical activity, colour, fluorescence, chemiluminescence, elementary composition, chemical structure, capability to react with other compounds, capability to elicit a response in a biological read out system (e.g., induction of a reporter gene) and the like. Values for said properties may serve as characteristic features and can be determined by techniques well known in the art. Moreover, the characteristic feature may be any feature which is derived from the values of the physical and/or chemical properties of a biomarker by standard operations, e.g., mathematical calculations such as multiplication, division or logarithmic calculus. Most preferably, the at least one characteristic feature allows the determination and/or chemical identification of the said at least one biomarker and its amount. Accordingly, the characteristic value, preferably, also comprises information relating to the abundance of the biomarker from which the characteristic value is derived. For example, a characteristic value of a biomarker may be a peak in a mass spectrum. Such a peak contains characteristic information of the biomarker, i.e. the m/z information, as well as an intensity value being related to the abundance of the said biomarker (i.e. its amount) in the sample.

As discussed before, each biomarker comprised by a sample may be, preferably, determined in accordance with the present invention quantitatively or semi-quantitatively. For quantitative determination, either the absolute or precise amount of the biomarker will be determined or the relative amount of the biomarker will be determined based on the value determined for the characteristic feature(s) referred to herein above. The relative amount may be determined in a case were the precise amount of a biomarker can or shall not be determined. In said case, it can be determined whether the amount in which the biomarker is present, is enlarged or diminished with respect to a second sample comprising said biomarker in a second amount. In a preferred embodiment said second sample comprising said biomarker shall be a calculated reference as specified elsewhere herein. Quantitatively analysing a biomarker, thus, also includes what is sometimes referred to as semi-quantitative analysis of a biomarker.

Thus, the determination of the amount of a lipid metabolite biomarker as referred to herein is preferably done by a compound separation step and a subsequent mass spectrometry step. Thus, determining as used in the method of the present invention, preferably, includes using a compound separation step prior to the analysis step. Preferably, said compound separation step yields a time resolved separation of the metabolites, in particular of the at least three lipid metabolite biomarkers, comprised by the sample. Suitable techniques for separation to be used preferably in accordance with the present invention, therefore, include all chromatographic separation techniques such as liquid chromatography (LC), high performance liquid chromatography (HPLC), gas chromatography (GC), thin layer chromatography, size exclusion or affinity chromatography. These techniques are well known in the art and can be applied by the person skilled in the art without further ado. Most preferably, LC and/or HPLC are chromatographic techniques to be envisaged by the method of the present invention. Suitable devices for such determination of biomarkers are well known in the art. Preferably, mass spectrometry is used in particular gas chromatography mass spectrometry (GC-MS), liquid chromatography mass spectrometry (LC-MS), direct infusion mass spectrometry or Fourier transform ion-cyclotroneresonance mass spectrometry (FT-ICR-MS), capillary electrophoresis mass spectrometry (CE-MS), high-performance liquid chromatography coupled mass spectrometry (HPLC-MS), quadrupole mass spectrometry, any sequentially coupled mass spectrometry, such as MS-MS or MSMS-MS, inductively coupled plasma mass spectrometry (ICP-MS), pyrolysis mass spectrometry (Py-MS), ion mobility mass spectrometry or time of flight mass spectrometry (TOF). More preferably, LC-MS, in particular LC-MS/MS, and most preferably HPLC-MS, in particular HPLCMS/MS, are used as described in detail below. Accordingly, the determination of the amounts to the at least three lipid metabolite biomarkers is preferably carried by HPLC-MS, in particular HPLC-MS/MS (high-performance liquid chromatography tandem mass spectrometry). The techniques described above are disclosed in, e.g., Nissen 1995, Journal of Chromatography A, 703: 37-57, U.S. Pat. No. 4,540,884 or U.S. Pat. No. 5,397,894, the disclosure content of which is hereby incorporated by reference.

As an alternative or in addition to mass spectrometry techniques, the following techniques may be used for compound determination: nuclear magnetic resonance (NMR), magnetic resonance imaging (MRI), Fourier transform infrared analysis (FT-IR), ultraviolet (UV) spectroscopy, refraction index (RI), fluorescent detection, radiochemical detection, electrochemical detection, light scattering (LS), dispersive Raman spectroscopy or flame ionisation detection (FID). These techniques are well known to the person skilled in the art and can be applied without further ado.

The method of the present invention shall be, preferably, assisted by automation. For example, sample processing or pre-treatment can be automated by robotics. Data processing and comparison is, preferably, assisted by suitable computer programs and databases. Automation as described herein before allows using the method of the present invention in high-throughput approaches.

As described above, said determining of the at least three lipid metabolite biomarkers can, preferably, comprise mass spectrometry (MS). Thus, a mass spectrometry step is carried out after the separation step (e.g. by LC or HPLC). Mass spectrometry as used herein encompasses all techniques which allow for the determination of the molecular weight (i.e. the mass) or a mass variable corresponding to a compound, i.e. a biomarker, to be determined in accordance with the present invention. Preferably, mass spectrometry as used herein relates to GC-MS, LC-MS, direct infusion mass spectrometry, FT-ICR-MS, CE-MS, HPLC-MS, quadrupole mass spectrometry, any sequentially coupled mass spectrometry such as MS-MS or MS-MS-MS, ICP-MS, Py-MS, TOF or any combined approaches using the aforementioned techniques. How to apply these techniques is well known to the person skilled in the art. Moreover, suitable devices are commercially available. More preferably, mass spectrometry as used herein relates to LC-MS and/or HPLC-MS, i.e. to mass spectrometry being operatively linked to a prior liquid chromatography separation step. Preferably, the mass spectrometry is tandem mass spectrometry (also known as MS/MS). Tandem mass spectrometry, also known as MS/MS involves two or more mass spectrometry step, with a fragmentation occurring in between the stages. In tandem mass spectrometry two mass spectrometers in a series connected by a collision cell. The mass spectrometers are coupled to the chromatographic device. The sample that has been separated by a chromatography is sorted and weighed in the first mass spectrometer, then fragmented by an inert gas in the collision cell, and a piece or pieces sorted and weighed in the second mass spectrometer. The fragments are sorted and weighed in the second mass spectrometer. Identification by MS/MS is more accurate.

In an embodiment, mass spectrometry as used herein encompasses quadrupole MS. Most preferably, said quadrupole MS is carried out as follows: a) selection of a mass/charge quotient (m/z) of an ion created by ionisation in a first analytical quadrupole of the mass spectrometer, b) fragmentation of the ion selected in step a) by applying an acceleration voltage in an additional subsequent quadrupole which is filled with a collision gas and acts as a collision chamber, c) selection of a mass/charge quotient of an ion created by the fragmentation process in step b) in an additional subsequent quadrupole, whereby steps a) to c) of the method are carried out at least once.

More preferably, said mass spectrometry is liquid chromatography (LC) MS such high performance liquid chromatography (H PLC) MS, in particular HPLC-MS/MS. Liquid chromatography as used herein refers to all techniques which allow for separation of compounds (i.e. metabolites) in liquid or supercritical phase. Liquid chromatography is characterized in that compounds in a mobile phase are passed through the stationary phase. When compounds pass through the stationary phase at different rates they become separated in time since each individual compound has its specific retention time (i.e. the time which is required by the compound to pass through the system). Liquid chromatography as used herein also includes HPLC. Devices for liquid chromatography are commercially available, e.g. from Agilent Technologies, USA. For examples, HPLC can be carried out with commercially available reversed phase separation columns with e.g. C8, C18 or C30 stationary phases. The person skilled in the art is capable to select suitable solvents for the HPLC or any other chromatography method as described herein. The eluate that emerges from the chromatography device shall comprise the biomarkers as referred to above.

A suitable solvent for elution for lipid chromatography can be determined by the skilled person. In an embodiment, the solvents for gradient elution in the HPLC separation consist of a polar solvent and a lipid solvent. Preferably, the polar solvent is a mixture of water and a water miscible solvent with an acid modifier. Examples of suitable organic solvents which are completely miscible with water include the C1-C3-alkanols, tetrahydrofurane, dioxane, C3-C4-ketones such as acetone and acetonitril and mixtures thereof, with methanol being particularly preferred. Additionally the lipid solvent is a mixture of the above mentioned solvents together with hydrophobic solvents from the groups consisting of dichloromethane (DCM), chloroform, tertiary butyl methyl ether (tBME or MTBE), ethyl ethanoate, and isooctane. Examples of acidic modifiers are formic acid or acidic acid. Preferred solvents for gradient elution are disclosed in the Examples section.

Gas chromatography which may be also applied in accordance with the present invention, in principle, operates comparable to liquid chromatography. However, rather than having the compounds (i.e. metabolites) in a liquid mobile phase which is passed through the stationary phase, the compounds will be present in a gaseous volume. The compounds pass the column which may contain solid support materials as stationary phase or the walls of which may serve as or are coated with the stationary phase. Again, each compound has a specific time which is required for passing through the column. Moreover, in the case of gas chromatography it is preferably envisaged that the compounds are derivatized prior to gas chromatography. Suitable techniques for derivatization are well known in the art. Preferably, derivatization in accordance with the present invention relates to methoxymation and trimethylsilylation of, preferably, polar compounds and transmethylation, methoxymation and trimethylsilylation of, preferably, nonpolar (i.e. lipophilic) compounds.

For mass spectrometry, the analytes in the sample are ionized in order to generate charged molecules or molecule fragments. Afterwards, the mass-to-charge of the ionized analyte, in particular of the ionized biomarkers, or fragments thereof is measured.

Thus, the mass spectrometry step preferably comprises an ionization step in which the biomarkers to be determined are ionized. Of course, other compounds present in the sample/elulate are ionizied as well. Ionization of the biomarkers can be carried out by any method deemed appropriate, in particular by electron impact ionization, fast atom bombardment, electrospray ionization (ESI), atmospheric pressure chemical ionization (APCI), matrix assisted laser desorption ionization (MALDI).

Preferably, the ionization step is carried out by atmospheric pressure chemical ionization (APCI). More preferably, the ionization step (for mass spectrometry) is carried out by electrospray ionization (ESI). Accordingly, the mass spectrometry is preferably ESI-MS (or if tandem MS is carried out: ESI-MS/MS). Electrospray is a soft ionization method which results in the formation of ions without breaking any chemical bonds.

Electrospray ionization (ESI) is a technique used in mass spectrometry to produce ions using an electrospray in which a high voltage is applied to the sample to create an aerosol. It is especially useful in producing ions from macromolecules because it overcomes the propensity of these molecules to fragment when ionized.

Preferably, the electrospray ionization is positive ion mode electrospray ionization. Thus, the ionization is preferably a protonation (or an adduct formation with positive charged ions such as NH₄⁺, Na⁺, or K⁺, in particular NH₄⁺). According a suitable cation, preferably, a proton (H⁺) is added to the biomarkers to be determined (and of course to any compound in the sample, i.e. in the eluate from the chromatography column). Therefore, the determination of the amounts of the at least three lipid metabolite biomarkers might be the determination of the amount of protonated biomarkers.

The person skilled in the art knows that the ionization step is carried out at the beginning of the mass spectrometry step. If tandem MS is carried out, the ionization, in particular the electrospray ionization, is carried out in the first mass spectrometry step.

The ionization of the biomarkers can be preferably carried out by feeding the liquid eluting from the chromatography column (in particular from the LC or HPLC column) directly to an electrospray. Alternatively the fractions can be collected and are later analyzed in a classical nanoelectrospray-mass spectrometry setup.

As set forth above, the mass spectrometry step is carried out after the separation step, in particular the chromatography step. In an embodiment, the eluate that emerges from the chromatography column (e.g. the LC or HPLC column) may be pre-treated prior to subjecting it to the mass spectrometry step. Preferably, ammonium buffer (most preferably ammonium formate or ammonium acetate) is added to the eluate in order to enhance the ionization efficiency in the electrospray process for some lipids such as ceramides and TAGs. Preferably, the ammonium formate buffer is dissolved in a solvent miscible with the gradient HPLC solvents (most preferably methanol).

In a preferred embodiment of the present invention, the at least three lipid metabolite biomarkers are determined together in a single measurement. In particular, it is envisaged to determine the amounts together in a single LC-MS (or LC-MS/MS), HPLC-MS (HPLC-MS/MS) measurement (i.e. run).

In an embodiment, the amounts of the at least three lipid metabolite biomarkers are determined as described in the Examples section.

For example, a blood, serum, or plasma sample (in particular a plasma sample) can be analyzed. The sample may be a fresh sample or a frozen sample. If frozen, the sample may be thawed at suitable temperature for a suitable time. An aliquot of the sample is then transferred to microcentrifuge tube and mixed with a suitable extraction solvent (e.g. 1:150 in methanol/dichloromethane (2:1 v/v). An internal standard may be added. Afterwards an extraction is done (e.g. for 5 minutes using a vortexer). After extraction, the sample may be centrifuged (e.g. at about 20.000 g). An aliquot of the supernatant (e.g. 200 μl) could be used for the quantification of the markers. The supernatant may be stored at −20° C. (up to three months).

How to determine the amount of the cardiac biomarkers as referred to herein is well known in the art. E.g., the cardiac biomarkers are determined as follows:

Total Cholesterol is typically tested in fasted patients (9-12 hours) using an enzymatic, colorimetric method standardised to NIST (National Institute of Standards and Technology). “Cholesterol” in the sample is first hydrolysed into free cholesterol and fatty acid by the enzyme cholesterol esterase. The free cholesterol is then oxidised by cholesterol oxidase to give cholestene-3-one and hydrogen peroxide.

Thus, a preferred embodiment of the method of the invention, the determination of the cardiac biomarker “total cholesterol” preferably comprises the steps of:

a) contacting the sample with cholesterol esterase under conditions and for a time sufficient to allow conversion into cholesterol;

b) contacting the sample comprising the cholesterol with cholesterol oxidase under conditions and for a time sufficient to allow generation of H₂O₂; and

c) enzymatically or chemically determining the amount of generated H₂O₂.

In another preferred embodiment of the method of the invention, the determination of the cardiac biomarker “total cholesterol” preferably comprises the steps of:

a) contacting the sample with cholesterol esterase under conditions and for a time sufficient to al-low conversion into cholesterol;

b) contacting the sample comprising the cholesterol with cholesterol dehydrogenase under conditions and for a time sufficient to allow generation of redox equivalents and, preferably, NADH
c) enzymatically or chemically determining the amount of generated redox equivalents.

In a preferred embodiment of the method of the invention, the determination of the amount of the cardiac biomarker “triglycerides” comprises the steps of:

a) contacting the sample with lipase under conditions and for a time sufficient to allow conversion into glycerol and free fatty acids;

b) contacting the sample comprising the glycerol with glycerokinase under conditions and for a time sufficient to allow conversion into glycerol-3-phosphate;

c) contacting the sample comprising the glycerol-3-phosphate with glycerophosphate oxidase under conditions and for a time sufficient to allow conversion into dihydroxyacetone phosphate and H₂O₂; and

d) enzymatically or chemically determining the amount of generated H₂O₂.

The enzymatic conversions described above generate redox equivalents in the form of H₂O₂. Said redox equivalents can be in turn detected in a further reaction by a peroxidase (such as a horseradish peroxidase) and a chromogenic substrate. The substrate is typically oxidized by the peroxidase using H₂O₂ as the oxidizing agent. The catalyzed reaction in the presence of H₂O₂, peroxidase, and the substrate typically results in a characteristic change that is detectable by spectrophotometric methods. E.g., the peroxidase catalyzes the conversion of chromogenic substrates into colored products, and produces light when acting on chemiluminescent substrates. For example, DAOS (N-ethyl-N-(2-hydroxy-3-sulfopropyl)-3,5-dimethoxyaniline) plus 4-aminoantipyrine in the presence of H₂O₂ and peroxidase results in the oxidative coupling of DAOS and 4-aminoantipyrine to form a blue colored compound. This colored compound can be e.g. detected by measuring the absorbance of light at about 590 nm. Alternatively, 10-acetyl-3,7-dihydroxyphenoxazine can be used as substrate for peroxidase that enables detection of H₂O₂. This non-fluorescent reagent reacts with H₂O₂ to produce resorufin, a fluorescent compound. In another alternative 4-aminophenazon plus 4-chlorphenol in the presence of H₂O₂ and peroxidase result in the formation of 4-(p-benzochinon-monoimino)-phenazon, a red colored product, which can be determined by photometric means.

In a preferred embodiment, the enzymes used for the enzymatic determination of the amount(s) of total cholesterol and triglycerides are non-human enzymes.

How to determine the amount of HDL-Cholesterol and LDL-Cholesterol is well known in the art. In an embodiment, the determination is based on centrifugation and subsequent enzymatic, colorimetric quantification. In another embodiment, the determination of the amount of HDL-Cholesterol and LDL-Cholesterol, respectively, is carried out by using specific antibodies for LDL-respectively HDL-specific apoproteins (immunological quantification). In an embodiment, the amounts of HDL-Cholesterol and/or LDL-Cholesterol are determined as described in Examples 7 or 8.

The centrifugation typically involves a precipitation reagent (e.g. containing 1,071 U/ml heparin, 500 mmol/l MnCl₂, and 12 mg/ml dextran sulfate) which is mixed with plasma or serum) and incubated for 5-10 min. Subsequently the sample is centrifugated at 2,000 to 105,000×g. The supernatant containing the HDL cholesterol is transferred into a new tube and the pellet containing the LDL-cholesterol is resuspended and dissolved (e.g. in PBS). Afterwards the amounts of HDL- and LDL-cholesterol can be determined enzymatically as described herein above for total cholesterol.

For an immunological quantification HDL respectively LDL specific antibodies are immobilized in a microtiter plate and the test samples is added to the wells and subsequently biotinylated HDL respectively LDL is added and then followed by washing with wash buffer. The antibodies are capable of binding apoproteins which are specific for LDL and HDL, respectively.

Streptavidin-Peroxidase Complex is added and unbound conjugates are washed away with wash buffer. TMB (3,3′,5,5′-Tetramethylbenzidine, a chromogenic substrate) is then used to visualize Streptavidin-Peroxidase enzymatic reaction. TMB is catalyzed by Streptavidin-Peroxidase to produce a blue color product that changes into yellow after adding acidic stop solution. The density of yellow coloration is inversely proportional to the amount of HDL respectively LDL captured in plate.

Lipoprotein subfraction biomarkers can be determined by a NMR spectroscopic assay (e.g. available from LipoScience, Raleigh, N.C.). In this assays, the characteristic NMR signals broadcast by lipoprotein particles of different size serve as the basis for quantification of these lipoprotein subfraction biomarkes in particle number concentration terms (moles of particles per liter). Mean LDL particle size (nm diameter) can be computed as the sum of the diameter of each subclass multiplied by its relative mass percentage as estimated from the amplitude of its NMR signal.

Alternatively, the lipoprotein subfraction biomarkers can be determined by ion mobility analysis. How to carry out such a ion mobility analysis is e.g. described in the publication Caulfield et al. (Clinical Chemistry August 2008 vol. 54 no. 8 1307-1316) which herewith is incorporated by reference with respect to the entire disclosure content. Ion mobility analysis preferably uses gas-phase electrophoresis to separate lipoproteins on the basis of size.

Ultracentrifugation and electrophoresis based methods are options for the measurement of small and medium LDL particles. A commercial available kit is provided by Randox: Randox sLDL-‘Ex-Seiken’ test is a direct method for the quantitative determination of small LDL particles using automated chemistry analysers capable of accommodating two-reagent assays or alternatives.

Alternatively, the amount of small and medium LDL particles can be determined by ion mobility analysis (see Caulfield et al.).

For a quantitative analysis of HDL subclasses auch as large HDL, an electrophoresis method on polyacrylamide gel (PAG) can be used. A preferred method is described in Oravec et al. (Neuro Endocrinol Lett. 2011; 32(4):502-9 which herewith is incorporated by reference with respect to the entire disclosure content.

Alternatively, the amount of large HDL particles can be determined by ion mobility analysis (see Caulfield et al.).

The ion mobility method directly determines particle numbers. Concentrations are preferably calculated from particle numbers as follows

The formula to convert from particle number to concentration is:

$$Pc = \frac{F . Pn {.10}^{18} . D .600}{A . M . Fr . R} , ,$$

where Pc_{—} particle concentration in nmol/L,

F=proportion of particles with a single charge (from Fuch distribution, see Wiedensohler A. An approximation of the bipolar charge distribution for particles in the submicron size range. J. Aerosol Sci 1988; 19:387-9.),

Pn=particle number counted in size range,

10¹⁸=mol to nmol and nL to L;

D=dilution of sample,

Fr=flow rate in nL/min,

600=0.1^{-s} bins per min,

A=Avogadro's number,

M=number of 0.1^{-s} bins included in size range

R=lipoprotein recovery after ultracentrifugation (50%).

The amount of apolipoprotein B (ApoB) is preferably determined by using an antibody or antigen binding fragment thereof which specifically binds said marker. For example, an immunoturbidimetric method may be used that measures increasing sample turbidity caused by the formation of insoluble immune antibody-biomarker complexes formed when said antibody or said antigen binding fragment thereof is added to the sample. In the presence of an antibody (or antigen binding fragment thereof) in excess, the amount of ApoB is determined as a function of turbidity.

The amount of Lp(a) is preferably determined by using an antibody or antigen binding fragment thereof which specifically binds said marker. For example, a latex-enhanced immunoturbidimetric method may be used for the determination of the marker. Lp(a) in the sample binds to the specific antibody (or antigen binding fragment thereof) which is coated on latex particles, and thereby causes agglutination. The degree of the turbidity caused by agglutination can be determined optically and is proportional to the amount of Lp(a) present in the sample.

The amount hsCRP (or CRP) is preferably determined by using an antibody or antigen binding fragment thereof which specifically binds said marker. For example, a latex-enhanced immunoturbidimetric method may be used for the determination of the marker. CRP in the sample binds to the specific antibody (or antigen binding fragment thereof) which is coated on latex particles, and thereby causes agglutination. The degree of the turbidity caused by agglutination can be determined optically and is proportional to the amount of CRP present in the sample.

The amount LP PLA2 is preferably determined by using an antibody or antigen binding fragment thereof which specifically binds said marker. For example, an immunoturbidimetric method may be used that measures increasing sample turbidity caused by the formation of insoluble immune antibody-biomarker complexes formed when said antibody or said antigen binding fragment thereof is added to the sample. In the presence of an antibody (or antigen binding fragment thereof) in excess, the amount of LP PLA2 is determined as a function of turbidity.

In a preferred embodiment of the present invention, the method according to the present invention further comprises the determination of the amount of BNP (brain natriuretic peptide, also known as B-type natriuretic peptide) or, in particular, NT-proBNP (N-terminus of the prohormone brain natriuretic peptide) in a sample from the subject and the thus determined amount of BNP or NT-proBNP is then compared to a reference. In a preferred embodiment the method according to the invention further comprises, in particular in step a) the determination of the amount of BNP (Brain natriuretic peptide, also known as B-type natriuretic peptide) or, in particular, NT-proBNP (N-terminus of the prohormone brain natriuretic peptide) in a sample from the subject. The thus determined amount of BNP or NT-proBNP is then compared to a reference (in step b) for BNP or NT-proBNP. Thus, the at least three lipid metabolite biomarkers, the at least one cardiac biomarker and BNP or NTproBNP might be determined at the same time (but preferably by varying assays). In another preferred embodiment of the present invention, the amount of NT-proBNP or BNP is determined in a sample in a further step c), and compared to a reference for NT-proBNP or BNP in a further step d). Based on steps b) and d) a cardiac disease is diagnosed. Thus, the may be a time gap between the determination of the amounts of the at least three lipid metabolite biomarkers and the at least one additional cardiac biomarker, and the determination of the amount of BNP or NT-proBNP (such as one day or one week).

Alternatively, the amount of NT-proBNP or BNP, or of the at least one additional cardiac biomarker can be derived from the medical record of the subject to be tested. Thus, the method of the present invention may comprise the step of providing and/or retrieving information on the amount of NT-proBNP or BNP, and/or the at least one additional cardiac biomarker (i.e. the value of this marker/these markers).

NT-proBNP and BNP are protein markers. The markers NT-proBNP and BNP are well known in the art. BNP is a 32-amino acid polypeptide secreted by the ventricles of the heart in response to excessive stretching of heart muscle cells (cardiomyocytes). NT-proBNP is a 76 amino acid N-terminal inactive protein that is cleaved from proBNP to release brain natriuretic peptide. BNP is the active hormone and has a shorter half-life than the respective inactive counterpart NT-proBNP. The structure of the human BNP and NT-proBNP has been described already in detail, see e.g., WO 02/089657, WO 02/083913.

It is to be understood that if the protein marker BNP or NTproBNP is determined, the protein marker is determined in addition to the at least three lipid metabolite biomarkers and in addition to the at least one cardiac biomarker as referred to herein in accordance with the method of the present invention.

Preferably, the amount is determined by using at least one antibody which specifically binds to the protein marker NT-proBNP or BNP. The at least one antibody forms a complex with the marker to determined (NT-proBNP or BNP). Afterwards the amount of the formed complex is measured. The complex comprises the marker and the antibody (which might be labelled in order to allow for a detection of the complex). The same preferably applies to the determination of the further protein biomarkers as referred to herein, in particular to cardiac biomarkers Lp(a), CRP/hsCRP, LP PLA2, and apolipoprotein B.

It is to be understood that the sample in which a protein biomarker is determined may require a pretreatment which differs from the pretreatment of the sample in which the other biomarkers, in particular lipid metabolite biomarkers are determined. For example, the proteins comprised by the sample in which a protein biomarker is determined are not precipitated. This is taken into account by the skilled person. Preferably, however, the amounts of the at least three lipid metabolite biomarkers and the at least one additional cardiac biomarker (and optionally of BNP or NT-proBNP) are measured in aliquots derived from the same sample. Alternatively, the amounts of the at least three lipid metabolite biomarkers and the at least one additional cardiac biomarker (and optionally of BNP or NT-proBNP) may be measured in aliquots derived from separate samples from the subject.

In an embodiment of the present invention, the amount of the protein marker NT-proBNP or BNP is determined for the marker panels in Table 2.

In addition, several lipid metabolite biomarker combinations that were tested in the studies underlying the present invention gave very accurate results for the diagnosis of heart failure even in the absence of the determination of NT-proBNP. So, in another embodiment of the present invention the method according to the present invention does not comprise the further determination of the amount of BNP or NT-proBNP. In particular, the method may not comprise the further determination of the amount of BNP or NT-proBNP and the comparison of the amount of BNP or NT-proBNP to a reference. Thus, the diagnosis of heart failure is not based on the determination of BNP and/or NT-proBNP. Accordingly, the method is a non-BNP and/or non-NT-proBNP based method.

Instead of NT-proBNP or BNP, or in addition to NT-proBNP or BNP, the present invention further envisages the determination of the amount of ANP (atrial natriuretic peptide) or NT-proANP (N-terminus of the prohormone brain natriuretic peptide). Alternatively, the amount of C-type natriuretic peptide or natriuretic peptide precursor C can be determined.

In an embodiment, the method of the present invention may further comprise carrying out a correction for confounders. Preferably, the values or ratios determined in a sample of a subject according to the present invention are adjusted for age, BMI, gender or existing diseases, e.g., the presence or absence of diabetes before comparing to a reference. Alternatively, the references can be derived from values or ratios which have likewise been adjusted for age, BMI, gender (see Examples). Such an adjustment can be made by deriving the references and the underlying values or ratios from a group of subjects the individual subjects of which are essentially identical with respect to these parameters to the subject to be investigated. Alternatively, the adjustment may be done by statistical calculations. Thus, a correction for confounders may be carried out. Preferred confounders are age, BMI (body mass index) and gender.

In another embodiment, a correction for confounders is not carried out. In a preferred embodiment, no correction for the confounders age, BMI and gender is carried out.

As set forth above, a correction for confounders may not be carried out, i.e. may be not required for a reliable diagnosis. This is advantageous since the diagnosis can be done even without the knowledge of certain patient's characteristics such as age, body mass index and gender. Thus, in an embodiment, the age, body mass index and/or gender, in particular the age and/or body mass index are not known (and thus are not taken into account for the diagnosis).

The term “reference” in connection with diagnostic methods is well known in the art. The reference in accordance with the present invention shall allow for the diagnosis of a cardiac disease. A suitable reference may be established by the skilled person without further ado. The reference to be applied may be an individual reference for each of the at least three lipid metabolite biomarkers and the at least one additional cardiac biomarker to be determined in the method of the present invention (and for BNP or NT-proBNP if determined). Accordingly, the amount of each of the at least three lipid metabolite biomarkers and the at least one additional cardiac biomarker (and of BNP or NT-proBNP if determined) as referred to in step a) of the method of the present invention is compared to a reference for each of the at least three lipid metabolite biomarkers and the at least one additional cardiac biomarker (and for BNP or NT-proBNP if determined). For example, if three biomarkers are determined in step a), three references (a reference for the first, a reference for the second, and a reference for the third marker) are applied in step b). A further reference for BNP or NT-proBNP might be used, if the amount of BNP or NT-proBNP is determined in step a). Based on the comparison of the amounts of the at least three lipid metabolite biomarkers and the at least one additional cardiac biomarker (and if BNP or NT-proBNP are determined, the amount of BNP or NT-proBNP), with the references, a diagnosis of a cardiac disease, i.e. whether the subject as referred to herein suffers from cardiac disease, or not, can be established.

The term “reference” refers to values of characteristic features which can be correlated to a medical condition, i.e. the presence or absence of the disease, diseases status or an effect referred to herein. Preferably, a reference is a threshold value for a biomarker of the at least three lipid metabolite biomarkers and the cardiac biomarkers as referred to in connection with the present invention whereby values found in a sample to be investigated which are higher than (or depending on the marker lower than) the threshold are indicative for the presence of a cardiac disease while those being lower (or depending on the marker higher than) are indicative for the absence of a cardiac disease.

The diagnostic algorithm may depend on the reference. If the reference amount is e.g. derived from a subject or group of subjects known to suffer from a cardiac disease, the presence of a cardiac disease is preferably indicated by amounts in the test sample which are essentially identical to the reference(s). If the reference amount is e.g. derived from an apparently healthy subject or group thereof, the presence of a cardiac disease is preferably indicated by amounts of the at least three lipid metabolite biomarkers and the at least one additional cardiac biomarker in the test sample which are different from (e.g. increased (“up”) or decreased (“down”) as compared to) the reference(s).

In accordance with the aforementioned method of the present invention, a reference (or references) is, preferably, a reference (or references) obtained from a sample from a subject or group of subjects known to suffer from a cardiac disease. In such a case, a value for each of the at least three lipid metabolite biomarkers and the at least one additional cardiac biomarker found in the test sample being essentially identical is indicative for the presence of the disease, i.e. of a cardiac disease. Moreover, the reference, also preferably, could be from a subject or group of subjects known not to suffer from a cardiac disease, preferably, an apparently healthy subject or group of subjects. In such a case, a value for each of the at least three lipid metabolite biomarkers and the at least additional cardiac biomarker found in the test sample being altered with respect to the reference is indicative for the presence of the disease. Alternatively, a value for each of the at least three lipid metabolite biomarkers and the at least additional cardiac biomarker found in the test sample being essentially identical with respect to the reference is indicative for the absence of the disease. The same applies mutatis mutandis for a calculated reference, most preferably the average or median, for the relative or absolute value of the biomarkers of a population of individuals comprising the subject to be investigated. The absolute or relative values of the biomarkers of said individuals of the population can be determined as specified elsewhere herein. How to calculate a suitable reference value, preferably, the average or median, is well known in the art. The population of subjects referred to before shall comprise a plurality of subjects, preferably, at least 5, 10, 50, 100, 1,000 or 10,000 subjects. It is to be understood that the subject to be diagnosed by the method of the present invention and the subjects of the said plurality of subjects are of the same species.

The value for a biomarker of the test sample and the reference values are essentially identical, if the values for the characteristic features and, in the case of quantitative determination, the intensity values are essentially identical. Essentially identical means that the difference between two values is, preferably, not significant and shall be characterized in that the values for the intensity are within at least the interval between 1^{st} and 99^{th} percentile, 5^{th} and 95^{th} percentile, 10^{th} and 90^{th} percentile, 20^{th} and 80^{th} percentile, 30^{th} and 70^{th} percentile, 40^{th} and 60^{th} percentile of the reference value, preferably, the 50^{th}, 60^{th}, 70^{th}, 80^{th}, 90^{th} or 95^{th} percentile of the reference value. Statistical test for determining whether two amounts or values are essentially identical are well known in the art and are also described elsewhere herein.

An observed difference for two values, on the other hand, shall be statistically significant. A difference in the relative or absolute value is, preferably, significant outside of the interval between 45^{th} and 55^{th} percentile, 40^{th} and 60^{th} percentile, 30^{th} and 70^{th} percentile, 20^{th} and 80^{th} percentile, 10^{th} and 90^{th} percentile, 5^{th} and 95^{th} percentile, 1^{st} and 99^{th} percentile of the reference value. Preferred changes and ratios of the medians are described in the Examples (see in particular Table 1A and 1B).

In a preferred embodiment the value for the characteristic feature can also be a calculated output such as score of a classification algorithm like “elastic net” as set forth elsewhere herein.

Preferably, the reference(s), i.e. a value or values for at least one characteristic feature (e.g. the amount) of the biomarkers or ratios thereof, will be stored in a suitable data storage medium such as a database and are, thus, also available for future assessments.

The term “comparing”, preferably, refers to determining whether the determined value of the biomarkers, or score (see below) is essentially identical to a reference or differs therefrom. Preferably, a value for a biomarker (or score) is deemed to differ from a reference if the observed difference is statistically significant which can be determined by statistical techniques referred to elsewhere in this description. If the difference is not statistically significant, the biomarker value and the reference are essentially identical. Based on the comparison referred to above, a subject can be assessed to suffer from cardiac disease or not.

For the specific lipid metabolite biomarkers referred to in this specification, preferred values for the changes in the relative amounts or ratios (i.e. the changes expressed as the ratios of the means) or the kind of direction of change (i.e. “up”- or “down” or increase or decrease resulting in a higher or lower relative and/or absolute amount or ratio) are indicated in the Table 1A and 1B in the Examples section. The ratio of means indicates the degree of increase or decrease, e.g., a value of 2 means that the amount is twice the amount of the biomarker compared to the reference. Moreover, it is apparent whether there is an “up-regulation” or a “down-regulation”. In the case of an “up-regulation” the ratio of the mean shall exceed 1.0 while it will be below 1.0 in case of a “down”-regulation. Accordingly, the direction of regulation can be derived from the Table as well. It will be understood that instead of the means, medians could be used as well.

For the specific cardiac biomarkers referred to in this specification, the kind of direction of change (i.e. “up”- or “down” or increase or decrease resulting in a higher or lower relative and/or absolute amount or ratio) are indicated in Table 3 in the Examples section.

“Up” preferably means that an increased amount of the biomarker (in particular as compared to the reference) is indicative for the presence of a cardiac disease, whereas a decreased amount of the biomarker (in particular as compared to the reference) is indicative for the absence of a cardiac disease.

“Down” preferably means that a decreased amount of the biomarker (in particular as compared to the reference) is indicative for the presence of a cardiac disease, whereas an increased amount of the biomarker (in particular as compared to the reference) is indicative for the absence of a cardiac disease.

The reference (s) for the lipid metabolite/cardiac biomarkers can be derived from healthy controt subjects. Healthy subjects are preferably subject known not to suffer from a cardiac disease. However, other references, in principle, could be established as well.

As can be derived from Table 1A and 1B, an increased amount of a triacylglyceride biomarker as compared to the reference shall be indicative for the presence of a cardiac disease (and thus for the diagnosis of a cardiac disease), whereas a decreased or an essentially identical amount as compared to the reference shall be indicative for the absence of a cardiac disease. Preferably, said reference is a reference derived from healthy control subjects (i.e. subjects known not to suffer from a cardiac disease), or from a healthy control subject.

With respect to the other further biomarkers as disclosed herein (i.e. of sphingomyelin biomarker, cholesterylester biomarker, phosphatidylcholine biomarker, ceramide biomarker and glutamic acid) a decreased amount of the biomarker as compared to the reference shall be indicative for the presence of a cardiac disease (and thus for the diagnosis that the patient does not suffer from cardiac disease), whereas an increased or an essentially identical amount as compared to the reference shall be indicative for the absence of cardiac disease. Preferably, said reference is a reference derived from healthy control subjects (i.e. subjects known not to suffer from cardiac disease), or from a healthy control subject.

If the amount of NT-proBNP or BNP is determined, an increased amount of this marker shall be indicative for the presence of cardiac disease (and thus for the diagnosis of cardiac disease), in particular heart failure, whereas a decreased or an essentially identical amount shall be indicative for the absence of a cardiac disease, in particular heart failure. In an embodiment, the reference amount for NT-proBNP is about 125 pg/mL, preferably in a serum or plasma sample, in particular for a human subject.

It is to be understood that the diagnostic algorithm might depend on the reference or references to be applied. However, this is taken into account by the skilled person who can establish suitable reference values and/or diagnostic algorithms based on the diagnosis provided herein. For example, the reference might be derived from a subject or group of subjects known to suffer from a cardiac disease. In this case the following applies: With respect to triacylglyceride biomarker, an amount (or amounts) of the triacylglyceride(s) in the sample from the subject which is (are) essential identical or which is (are) increased as compared to the reference is indicative for the presence of a cardiac disease. With respect to the remaining biomarkers that can determined, an amount (or amounts) of the remaining biomarker(s) in the sample from the subject which is (are) decreased or essentially identical as compared to the reference is (are) indicative for the presence of a cardiac disease.

The comparison is, preferably, assisted by automation. For example, a suitable computer program comprising algorithms for the comparison of two different data sets (e.g., data sets comprising the values of the characteristic feature(s)) may be used. Such computer programs and algorithms are well known in the art. Notwithstanding the above, a comparison can also be carried out manually.

In the context of step b) of the present invention, the amounts of a group of biomarkers as referred to in step a) of the methods of the present invention shall be compared to a reference or references. Thereby, the presence or absence of a disease as referred to herein is diagnosed. In an embodiment references for the individual determined biomarkers, i.e. references for each of biomarkers as referred to in step a) are applied. However, it is also envisaged to calculate a score (in particular a single score) based on the amounts of the at least three lipid metabolite biomarkers and the at least one additional cardiac biomarker as referred to in step a) of the method of the present invention, i.e. a single score, and to compare this score to a reference score. Preferably, the score is based on the amounts of the at least three lipid metabolite biomarkers and the at least one additional cardiac biomarker in the sample from the test subject, and, if NT-proBNP or BNP is determined, on the amounts of the at least three lipid metabolite biomarkers, the amount(s) of the at least one additional cardiac biomarker and the amount of NT-proBNP or BNP in the sample from the test subject. For example, if the amounts of the biomarkers of panel 1 and Lp (a) are determined, the calculated score is based on the amounts of SM23, OSS2, PC4 and Lp (a) in the sample from the test subject. If additionally NT-proBNP is determined, the score is based on the amount of SM23, OSS2, PC4, Lp (a) and NT-proBNP of the test subject.

The calculated score combines information on the amounts of the at least three lipid metabolite biomarkers and the at least one additional cardiac biomarker. Moreover, in the score, the biomarkers are, preferably, weighted in accordance with their contribution to the establishment of the diagnosis. Based on the combination of biomarkers applied in the method of the invention, the weight of an individual biomarker may be different.

The score can be regarded as a classifier parameter for diagnosing a cardiac disease. In particular, it enables the person who provides the diagnosis based on a single score based on the comparison with a reference score. The reference score is preferably a value, in particular a cut-off value which allows for differentiating between the presence of a cardiac disease and the absence of a cardiac disease in the subject to be tested. Preferably, the reference is a single value. Thus, the person does not have to interpret the entire information on the amounts of the individual biomarkers.

Using a scoring system as described herein, advantageously, values of different dimensions or units for the biomarkers may be used since the values will be mathematically transformend into the score. Accordingly, values for absolute concentrations may be combined in a score with peak area ratios.

Thus, in a preferred embodiment of the present invention, the comparison of the amounts of the biomarkers to a reference as set forth in step b) of the method of the present invention encompasses step b1) of calculating a score based on the determined amounts of the biomarkers as referred to in step a), and step b2) of comparing the, thus, calculated score to a reference score. More preferably, a logistic regression method is used for calculating the score and, most preferably, said logistic regression method comprises elastic net regularization.

Alternatively, the amount of each of the at least three lipid metabolite biomarkers and of the at least one additional cardiac biomarker is compared to a reference, wherein the result of this comparison is used for the calculation of a score (in particular a single score), and wherein said score is compared to a reference score.

Thus, the present invention, in particular, a method for diagnosing a cardiac disease in a subject comprising the steps of:

a) determining in a sample of a subject as referred to herein the amounts of at least three lipid metabolite biomarkers and of at least one additional cardiac biomarker as referred to above; and

b1) calculating a score based on the determined amounts of the at least three lipid metabolite biomarkers and the at least one additional cardiac biomarker as referred to in step a), and

b2) comparing the, thus, calculated score to a reference score, whereby a cardiac disease is to be diagnosed.

As set forth elsewhere herein, the aforementioned method may further comprise in step a) the determination of the amount of BNP or NT-proBNP. The amount of BNP or NT-proBNP may contribute to the score calculated in step b). Accordingly, the method comprises the following steps:

a) determining in a sample of a subject as referred to herein the amounts of at least three lipid metabolite biomarkers and of at least one additional cardiac biomarker as referred to above and the amount of BNP or NT-proBNP; and

b1) calculating a score based on the determined amounts of the at least three lipid metabolite biomarkers and the at least one additional cardiac biomarker and on the amount of BNP or NT-proBNP as referred to in step a), and

b2) comparing the, thus, calculated score to a reference score, whereby a cardiac disease is to be diagnosed.

As set forth elsewhere herein, the amount of NT-proBNP or BNP can be also derived from the medical record of the subject to be tested. In this case, it is not required to the determine the amount of this marker in step a).

Alternatively, the amount of BNP or NT-proBNP may not contribute to the score calculated in step b1). Accordingly, the method comprises the following steps:

a) determining in a sample of a subject as referred to herein the amounts of at least three lipid metabolite biomarkers and of at least one additional cardiac biomarker as referred to above and the amount of BNP or NT-proBNP; and

b1) calculating a score based on the determined amounts of the at least three lipid metabolite biomarkers and of at least one additional cardiac biomarker, and

b2) comparing the, thus, calculated score to a reference score, and comparing the amount of BNP or NT-proBNP to a reference, whereby a cardiac disease is to be diagnosed.

Alternatively, a score may calculated based on the at least three lipid metabolite biomarkers and on NT-proBNP or BNP.

Various other scores could be formed. As set forth above, the present invention is based on the determination of the at least three lipid metabolite biomarkers, at least one additional cardiac biomarker, and optionally NT-proBNP or BNP. In principle, any proportion of the determined biomarkers can be used for the calculation of a score, which is the compared to a reference score. The amount(s) remaining biomarkers, i.e. the biomarker which are not used for the calculation of the score, are compared to a reference.

As set forth elsewhere herein, the amount of NT-proBNP or BNP or the amount of the additional cardiac biomarker can be also derived from the medical record of the subject to be tested. In this case, it is not required to the determine the amount of these markers in step a).

Preferably, the reference score shall allow for differentiating whether a subject suffers from a cardiac disease as referred to herein, or not. Preferably, the diagnosis is made by assessing whether the score of the test subject is above or below the reference score. Thus, in an embodiment it is not necessary to provide an exact reference score. A relevant reference score can be obtained by correlating the sensitivity and specificity and the sensitivity/specificity for any score. A reference score resulting in a high sensitivity results in a lower specificity and vice versa. Preferably, the reference score is based on the same markers (e.g. the at least three lipid metabolite biomarkers, the at least one additional cardiac biomarker and NT-proBNP) as the score.

The reference score may be a “Cut-Off” value which allows for differentiating between the presence and the absence of a cardiac disease in the subject.

In accordance with the present invention, a reference score is, preferably, a reference score obtained from a sample from a subject or group of subjects known to suffer from a cardiac disease. In such a case, a score in the test sample being essentially identical is indicative for the presence of the disease, i.e. of a cardiac disease. Moreover, the reference score, also preferably, could be from a subject or group of subjects known not to suffer from a cardiac disease, preferably, an apparently healthy subject or a group of apparently healthy subjects. In such a case, a score in the test sample being altered, in particular increased, with respect to the reference score is indicative for the presence of the disease. Alternatively, a score in the test sample being essentially identical to said reference score is indicative for the absence of the disease.

Preferably, the score is calculated based on a suitable scoring algorithm. Said scoring algorithm, preferably, shall allow for differentiating whether a subject suffers from a disease as referred to herein, or not, based on the amounts of the biomarkers to be determined.

Preferably, said scoring algorithm has been previously determined by comparing the information regarding the amounts of the individual biomarkers as referred to in step a) in samples from patients suffering from a cardiac disease as referred to herein and from patients not suffering from a cardiac disease. Accordingly, step b) may also comprise step b0) of determining or implementing a scoring algorithm. Preferably, this step is carried out prior steps b1) and b2).

In an embodiment the reference score is calculated such that an increased amount of the score of the test subject as compared to the reference score is indicative for the presence of a cardiac disease, and/or a decreased amount of the score of the test subject as compared to the reference score is indicative for the absence of a cardiac disease. In particular, the score may be a cut-off value.

In a preferred embodiment of the present invention (e.g. of the methods, devices, uses etc.), the reference score is a single cut-off value. Preferably, said value allows for allocating the test subject either into a group of subjects suffering from a cardiac disease or a into a group of subjects not suffering from a cardiac disease. Preferably, a score for a subject lower than the reference score is indicative for the absence of a cardiac disease in said subject (and thus can be used for ruling out a cardiac disease), whereas a score for a subject larger than the reference score is indicative for the presence of a cardiac disease in said subject (and thus can be used for ruling in a cardiac disease).

In another preferred embodiment of the present invention (e.g. of the methods, devices, uses etc.), the reference score is a reference score range. In this context, a reference score range indicative for the presence of a cardiac disease, a reference score range indicative for the absence of a cardiac disease, or two reference score ranges (i.e. a reference score range indicative for the presence of a cardiac disease and a reference score range indicative for the absence of a cardiac disease) can be applied.

Preferably, the score of a subject is compared to the reference score range (or ranges). Preferably, the absence of a cardiac disease is diagnosed, if the score is within the reference score range indicative for the absence of a cardiac disease. A score which is not within the reference score range indicative for the absence of a cardiac disease is, preferably, indicative for the presence of a cardiac disease. In this case, the presence of a cardiac disease is diagnosed.

Alternatively, the presence of a cardiac disease is diagnosed, if the score is within the reference score range indicative for the presence of a cardiac disease. A score which is not within the reference score range indicative for the presence of a cardiac disease is, preferably, indicative for the absence of a cardiac disease. In this case, the absence of a cardiac disease is diagnosed.

A suitable scoring algorithm can be determined with the at least three lipid metabolite biomarkers and the at least one additional cardiac biomarker referred to in step a) by the skilled person without further ado (and optionally of BNP or NT-proBNP). E.g., the scoring algorithm may be a mathematical function that uses information regarding the amounts of the at least three lipid metabolite biomarkers and the at least one additional cardiac biomarker (and optionally of BNP or NT-proBNP) in a cohort of subjects suffering from a cardiac disease and not suffering from a cardiac disease. Methods for determining a scoring algorithm are well known in the art and including Significance Analysis of Microarrays, Tree Harvesting, CART, MARS, Self Organizing Maps, Frequent Item Set, Bayesian networks, Prediction Analysis of Microarray (PAM), SMO, Simple Logistic Regression, Logistic Regression, Multilayer Perceptron, Bayes Net, Naïve Bayes, Naïve Bayes Simple, Naïve Bayes Up, IB1, lbk, Kstar, LWL, AdaBoost, ClassViaRegression, Decorate, Multiclass Classifier, Random Committee, j48, LMT, NBTree, Part, Random Forest, Ordinal Classifier, Sparse Linear Programming (SPLP), Sparse Logistic Regression (SPLR), Elastic net, Support Vector Machine, Prediction of Residual Error Sum of Squares (PRESS), Penalized Logistic Regression, Mutual Information. Preferably, the scoring algorithm is determined with or without correction for confounders as set forth elsewhere herein.

In an embodiment, the scoring algorithm is determined with an elastic net with at least three lipid metabolite biomarkers and the at least one additional cardiac biomarker, and optionally with BNP or NT-proBNP (see also Examples section).

Typically, a classification algorithm such as those implementing the elastic net method may be used for scoring (Zou 2005, Journal of the Royal Statistical Society, Series B: 301-320, Friedman 2010, J. Stat. Sotw. 33). Thus, the score for a subject can be, preferably, calculated with a logistic regression model fitted, e.g., by using the elastic net algorithm such as implemented in the R package glmnet. More specifically, the score may be calculated by the following formula

$$p = \frac{1}{1 + {e}^{- ( {w}_{0} + {Σ}_{i = 1}^{n} {w}_{i} {\hat{x}}_{i} )}}$$

or a mathematically equivalent formula,

with the feature \hat{x}ᵢ being

$${\hat{x}}_{i} = \frac{{x}_{i} - {m}_{i}}{{s}_{i}}$$

wherein xᵢ are the log-transformed measurement values, e.g., peak area ratios and/or concentration values, and mᵢ, sᵢ are feature specific scaling factors and wᵢ are the coefficients of the model [w₀, intercept; w₁, coefficient for the first feature (e.g. NT-proBNP, or any one of the lipid metabolite or of any of the cardiac biomarkers as referred to herein); w₂ . . . wₙ, coefficients for the further features; n, number of feautures in the panel].

A score larger than the reference score is indicative for a subject who suffers from a cardiac disease, whereas a score lower than (or equal to) the reference score is indicative for a subject who does not suffer from a cardiac disease.

The reference scores e.g. can be determined to maximize the Youden index for the detection of a cardiac disease.

The at least three lipid metabolite biomarkers as specified above and the at least one additional cardiac biomarker in a sample can, in principle, be used for assessing whether a subject suffers from a cardiac disease, or not. This is particularly helpful for an efficient diagnosis of the disease as well as for improving of the pre-clinical and clinical management of a cardiac disease as well as an efficient monitoring of patients.

The definitions and explanations of the terms made above apply mutatis mutandis for the following embodiments of the present invention (e.g. to the kits, devices, uses, further methods etc.) except specified otherwise herein below.

By carrying out the method of the present invention, a subject can be identified who is in need for a therapy of a cardiac disease. Accordingly, the present invention relates to a method for identifying whether a subject is in need for a therapy of a cardiac disease or a change of therapy comprising the steps of the methods of the present invention and the further step of identifying a subject in need if a cardiac disease is diagnosed.

The phrase “in need for a therapy of a cardiac disease” as used herein means that the disease in the subject is in a status where therapeutic intervention is necessary or beneficial in order to ameliorate or treat a cardiac disease or the symptoms associated therewith. Accordingly, the findings of the studies underlying the present invention do not only allow diagnosing a cardiac disease in a subject but also allow for identifying subjects which should be treated by a a cardiac disease therapy or whose a cardiac disease therapy needs adjustment. Once the subject has been identified, the method may further include a step of making recommendations for a therapy of a cardiac disease.

The therapy may depend on the type of cardiac disease.

If the cardiac disease is heart failure, preferably the following applies:

A therapy of heart failure as used in accordance with the present invention, preferably, relates to a therapy which comprises or consists of the administration of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, Vitamin-K-Antagonists, polypeptides of the protein S100 family (as disclosed by DE000003922873A1, DE000019815128A1 or DE000019915485A1 hereby incorporated by reference), natriuretic peptides such as BNP (Nesiritide (human recombinant Brain Natriuretic Peptide—BNP)) or ANP. Alternationally or alternatively, the therapy may include lifestyle changes such as reduced intake of dietary fat, regular participation in light or moderate exercise, salt restriction and/or cessation of smoking.

As a rule, patients are preferably treated with medication as recommended by the guidelines of the European Society of Cardiology (Ref: European Heart Journal (2012), 33:1787-1847).

In another embodiment the patients are treated as recommended by the 2013 ACCF/AHA guidelines (see Circulation. 2013; 128: e240-e327).

Preferably, the therapy comprises the administration of Mineralocorticoid/Aldosteron Antagonists and/or ACE Inhibitors, if the subject is diagnosed to suffer from HFrEF. Further envisaged is the treatment with a beta blocker.

Moreover, the subject may be treated with angiotensin receptor blockers (ARBs), ivabradine, digoxin and other digitalis glycosides, hydralazine and isosorbide dintrate (vasodilators) and omega-3 polyunsaturated fatty acids.

Preferably, the therapy comprises the administration of Diuretics, Aldosteron Antagonists and/or ACE Inhibitors, if the subject is diagnosed to suffer from DCMP.

Preferably, the therapy comprises the administration of Diuretics, Aldosteron Antagonists and/or ACE Inhibitors, if the subject is diagnosed to suffer from ICMP. Also preferred are Vitamin-K-antagonists and antiplatelet agents.

If the subject suffers from HFpEF, the therapy preferably comprises the administration of diuretics,

ACE inhibitor, receptor blockers (ARBs) and/or beta blockers.

If the cardiac disease is coronary artery disease, the therapy is preferably drug based therapy. Preferably, the therapy comprises or consists of the administration of at least one drug selected from the group consisting of: Cholesterol-modifying medications such as statins, beta blockers, Angiotensin-converting enzyme (ACE) inhibitors, angiotensin II receptor blockers (ARBs), Nitroglycerin, and acetylsalicylic acid. Alternationally or alternatively, the therapy may include lifestyle changes such as reduced intake of dietary fat, regular participation in light or moderate exercise, salt restriction and/or cessation of smoking.

The present invention further relates to a method for determining whether a therapy against heart failure is successful in a subject comprising the steps of the methods of the present invention and the further step of determining whether a therapy is successful if no heart failure is diagnosed.

It is to be understood that a therapy will be successful if a cardiac disease or at least some symptoms thereof can be treated or ameliorated compared to an untreated subject. Moreover, a therapy is also successful as meant herein if the disease progression can be prevented or at least slowed down compared to an untreated subject.

In a preferred embodiment of the method of the invention, the determination of the at lipid metabolite biomarkers is achieved by mass spectroscopy techniques (preferably GC-MS and/or LC-MS), NMR or others referred to herein above. In such cases, preferably, the sample to be analyzed is pretreated. Said pretreatment, preferably, includes obtaining of the at least one preferably the at least three lipid metabolite biomarker from sample material, e.g., plasma or serum may be obtained from whole blood or the at least one, preferably the at least three lipid metabolite biomarkers may even be specifically extracted from sample material. Moreover, for GC-MS, further sample pretreatment such as derivatization of the at least one biomarker is, preferably, required. Furthermore, pretreatment also, preferably, includes diluting sample material and adjusting or normalizing the concentration of the components comprised therein. To this end, preferably, normalization standards may be added to the sample in predefined amounts which allow for making a comparison of the amount of the at least one biomarker and the reference and/or between different samples to be analyzed. For example, one standard for each class of biomarkers may be added in order to allow for a normalization, e.g one standard for triacylglyerides, one standard for sphingomyelins etc. In another preferred embodiment the quantification of SM biomarker is achieved by adding commercially available sphingomyeline standards with a different chain length than the target metabolites based on the observation that the detector response is the same. In a further preferred embodiment the calibration solutions are prepared in delipidized plasma (commercially available) to simulate a matrix as close as possible to real plasma.

The method of the present invention, in a preferred embodiment, furthermore further comprises a step of recommending and/or managing the subject according to the result the diagnosis established in step b). Such a recommendation may, in an aspect, be an adaptation of life style, nutrition and the like aiming to improve the life circumstances, the application of therapeutic measures as set forth elsewhere herein in detail, and/or a regular disease monitoring.

In another preferred embodiment of the aforementioned method, step b) is carried out by an evaluation unit as set forth elsewhere herein.

Further, the present invention also in an aspect pertains to a method of treating a cardiac disease comprising the steps a) and b) of the method for diagnosing a cardiac disease, and the further step c) of treating the subject in case the subject is diagnosed to suffer from a cardiac disease. The method may also comprise the step b1) of selecting a subject who suffers from a cardiac disease. In an embodiment, step b1) is carried out after step b), but before step c). For example, the subject may be treated, if the subject suffers from a cardiac disease e.g. symptomatic or asymptomatic heart failure.

Further, the present invention also in an aspect pertains to a method of treating a cardiac disease comprising the steps of the method for identifying whether a subject is in need for a therapy of a cardiac disease or a change of therapy comprising the steps of the methods of the present invention, the further step of identifying a subject in need if a cardiac disease is diagnosed and the further step of treating the subject accordingly.

The definitions and explanations of the terms made above apply mutatis mutandis for the following embodiments of the present invention except specified otherwise herein below.

The aforementioned methods for the determination of the at least three lipid metabolite biomarkers and the at least one additional cardiac biomarker can be implemented into a device. A device as used herein shall comprise at least the aforementioned means. Moreover, the device, preferably, further comprises means for comparison and evaluation of the detected characteristic feature(s) of the at least three lipid metabolite biomarker and the at least one additional cardiac biomarker and, also preferably, the determined signal intensity. The means of the device are, preferably, operatively linked to each other. How to link the means in an operating manner will depend on the type of means included into the device. For example, where means for automatically qualitatively or quantitatively determining the biomarker are applied, the data obtained by said automatically operating means can be processed by, e.g., a computer program in order to facilitate the assessment. Preferably, the means are comprised by a single device in such a case. Said device may accordingly include an analyzing unit for the biomarker and a computer unit for processing the resulting data for the assessment. Preferred devices are those which can be applied without the particular knowledge of a specialized clinician, e.g., electronic devices which merely require loading with a sample.

Alternatively, the methods for the determination of the biomarkers can be implemented into a system comprising several devices which are, preferably, operatively linked to each other. Specifically, the means must be linked in a manner as to allow carrying out the method of the present invention as described in detail above. Therefore, operatively linked, as used herein, preferably, means functionally linked. Depending on the means to be used for the system of the present invention, said means may be functionally linked by connecting each mean with the other by means which allow data transport in between said means. A preferred system comprises means for determining biomarkers. Means for determining biomarkers as used herein encompass means for separating biomarkers, such as chromatographic devices, and means for metabolite determination, such as mass spectrometry devices. Suitable devices have been described in detail above. Preferred means for compound separation to be used in the system of the present invention include chromatographic devices, more preferably devices for liquid chromatography, HPLC, and/or gas chromatography. Preferred devices for compound determination comprise mass spectrometry devices, more preferably, GC-MS, LC-MS, direct infusion mass spectrometry, FT-ICR-MS, CE-MS, HPLC-MS, quadrupole mass spectrometry, sequentially coupled mass spectrometry (including MS-MS or MS-MS-MS), ICP-MS, Py-MS or TOF. The separation and determination means are, preferably, coupled to each other. Most preferably, LC-MS, in particular HPLC-MS, and/or GC-MS are used in the system of the present invention as described in detail elsewhere in the specification. Further comprised shall be means for comparing and/or analyzing the results obtained from the means for determination of the at least three lipid metabolite biomarkers and the at least one additional cardiac biomarker (and optionally of NT-proBNP or BNP). The means for comparing and/or analyzing the results may comprise at least one databases and an implemented computer program for comparison of the results. Preferred embodiments of the aforementioned systems and devices are also described in detail below.

Therefore, the present invention relates to a diagnostic device comprising:

a) an analysing unit comprising at least one detector for the at least three lipid metabolite biomarkers and at least one detector for and the at least one additional cardiac biomarker in connection with the present invention detected by the at least one detector, and, operatively linked thereto;

b) an evaluation unit comprising a computer comprising tangibly embedded a computer program code for carrying out a comparison of the determined amounts of the at least three lipid metabolite biomarkers and the at least one additional cardiac biomarker with the reference amounts, and a data base comprising said reference amounts for the said biomarkers, whereby it will be diagnosed whether a subject suffers from a cardiac disease.

Alternatively, the evaluation unit under b) comprises a computer comprising tangibly embedded a computer program code for calculating a score based on the determined amounts of the at least three lipid metabolite biomarkers and and the at least one additional cardiac biomarker, and for carrying out a comparison of the calculated score and the reference score, wherein said evaluation unit further comprises a data base comprising said reference score, whereby it will be diagnosed whether a subject suffers from a cardiac disease.

The terms “score” and “reference score” are determined elsewhere herein.

In an embodiment, the device further comprises at least one further analysing unit comprising at least one detector for NT-proBNP and/or BNP, wherein said further analyzing unit is adapted for determining the amounts BNP and/or NT-proBNP detected by the at least one detector.

Thus, the present invention relates to a diagnostic device comprising:

a) an analysing unit comprising at least one detector for the at least three lipid metabolite biomarkers and at least one detector for and the at least one additional cardiac biomarker, wherein said analyzing unit is adapted for determining the amounts of the said biomarkers detected by the at least one detector, and, optionally, at least one further analysing unit comprising at least one detector for NT-proBNP and/or BNP, wherein said further analyzing unit is adapted for determining the amounts BNP and/or NT-proBNP detected by the at least one detector for NT-proBNP and/or BNP,

and operatively linked thereto;

b) an evaluation unit comprising a computer comprising tangibly embedded a computer program code for carrying out a comparison of the determined amounts of the at least three lipid metabolite biomarkers and the at least one additional cardiac biomarker and, optionally, of BNP or NT-proBNP, and the reference amounts and a data base comprising said reference amounts for the said biomarkers, whereby it will be diagnosed whether a subject suffers from a cardiac disease.

Alternatively, the evaluation unit under b) comprises a computer comprising tangibly embedded a computer program code for calculating a score based on the determined amounts of the at least three lipid metabolite biomarkers, the at least one additional cardiac biomarker and of BNP and/or NT-proBNP, and for carrying out a comparison of the calculated score and the reference score, wherein said evaluation unit further comprises a data base comprising said reference score, whereby it will be diagnosed whether a subject suffers from a cardiac disease.

Preferably, the devices are adapted to carry out the method of the present invention.

Preferably, the computer program code is capable of executing steps of the method of the present invention as specified elsewhere herein in detail. Accordingly, the device can be used for diagnosing a cardiac disease as specified herein based on a sample of a subject.

In a preferred embodiment, the device comprises a further database comprising the kind of regulation and/or fold of regulation values indicated for the respective biomarkers in any one of Tables 1A and/or 1B, and/or the kind of regulation indication for the respective cardiac biomarkers in Table 3 and a further tangibly embedded computer program code for carrying out a comparison between the determined kind of regulation and/or fold of regulation values and those comprised by the database. In another preferred embodiment, the device comprises a further database comprising the kind of regulation and/or fold of regulation values indicated for the score(s) calculated based on the amounts of the at least three lipid metabolite biomarkers and the at least one additional cardiac biomarker, and a further tangibly embedded computer program code for carrying out a comparison between the determined kind of regulation and/or fold of regulation values for the score and those comprised by the database.

Furthermore, the present invention relates to a data collection comprising characteristic values of the at least three lipid metabolite biomarkers and the at least one additional cardiac biomarker being indicative for a medical condition or effect as set forth above (i.e. diagnosing a cardiac disease in a subject). Furthermore, the present invention relates to a data collection comprising characteristic values of scores calculated based on the amounts of the at least three lipid metabolite biomarkers and the at least one additional cardiac biomarker as set forth herein being indicative for a medical condition or effect as set forth above (i.e. diagnosing a cardiac disease in a subject).

The term “data collection” refers to a collection of data which may be physically and/or logically grouped together. In one embodiment the physical or logical grouping is realized by classification approaches like elastic net, random forest, penalized logistic regression or others known by the person skilled in the art. Accordingly, the data collection may be implemented in a single data storage medium or in physically separated data storage media being operatively linked to each other. Preferably, the data collection is implemented by means of a database. Thus, a database as used herein comprises the data collection on a suitable storage medium. Moreover, the database, preferably, further comprises a database management system. The database management system is, preferably, a network-based, hierarchical or object-oriented database management system. Furthermore, the database may be a federal or integrated database. More preferably, the database will be implemented as a distributed (federal) system, e.g. as a Client-Server-System. More preferably, the database is structured as to allow a search algorithm to compare a test data set with the data sets comprised by the data collection. Specifically, by using such an algorithm, the database can be searched for similar or identical data sets being indicative for a medical condition or effect as set forth above (e.g. a query search). Thus, if an identical or similar data set can be identified in the data collection, the test data set will be associated with the said medical condition or effect. Consequently, the information obtained from the data collection can be used, e.g., as a reference for the methods of the present invention described above. More preferably, the data collection comprises characteristic values of all at least three lipid metabolite biomarkers and the at least one additional cardiac biomarker recited above. Also preferably, the data collection comprises scores for the at least three lipid metabolite biomarkers and the at least one additional cardiac biomarker as set forth above.

In light of the foregoing, the present invention encompasses a data storage medium comprising the aforementioned data collection.

The term “data storage medium” as used herein encompasses data storage media which are based on single physical entities such as a CD, a CD-ROM, a hard disk, a flash storage medium, optical storage media, or a diskette. Moreover, the term further includes data storage media consisting of physically separated entities which are operatively linked to each other in a manner as to provide the aforementioned data collection, preferably, in a suitable way for a query search.

The present invention also relates to a system comprising:

(a) means for comparing characteristic values of the biomarkers of the at least three lipid metabolite biomarkers and the at least one additional cardiac biomarker. or a score (based on the amounts of said biomarkers) of a sample operatively linked to

(b) a data storage medium as described above.

The system may further comprise means for comparing characteristic values of BNP or NT-proBNP (or a score based thereon).

In another embodiment the present invention also relates to a system comprising:

(a) means for comparing a score based on the amounts of the at least three lipid metabolite biomarkers, the at least one additional cardiac biomarker and of BNP or NT-proBNP of a sample operatively linked to

(b) a data storage medium as described above.

The term “system” as used herein relates to different means which are operatively linked to each other. Said means may be implemented in a single device or may be physically separated devices which are operatively linked to each other. The means for comparing characteristic values of biomarkers, preferably, based on an algorithm for comparison as mentioned before. The data storage medium, preferably, comprises the aforementioned data collection or database, wherein each of the stored data sets being indicative for a medical condition or effect referred to above. Thus, the system of the present invention allows identifying whether a test data set is comprised by the data collection stored in the data storage medium. Consequently, the methods of the present invention can be implemented by the system of the present invention.

In a preferred embodiment of the system, means for determining characteristic values of biomarkers of a sample are comprised. The term “means for determining characteristic values of biomarkers” preferably relates to the aforementioned devices for the determination of metabolites such as mass spectrometry devices, NMR devices or devices for carrying out chemical or biological assays for the biomarkers.

Moreover, the present invention relates to a diagnostic means comprising means for the determination of at the at least three lipid metabolite biomarkers and the at least one additional cardiac biomarker as referred to in connection with the method of the present invention.

The diagnostic means may further comprise means for the determination of BNP or NT-proBNP, in particular said determination is based on a determination with respective antibodies, and thus said means shall comprise at least one antibody, or fragment thereof, which binds specifically BNP or NT-proBNP. Thus, said means shall allow for the determination of NT-proBNP or BNP. Preferably, said means are adapted to carry out an immunoassay for determining the amount of the marker.

The term “diagnostic means”, preferably, relates to a diagnostic device, system or biological or chemical assay as specified elsewhere in the description in detail.

The expression “means for the determination of at least three biomarkers” refers to devices or agents which are capable of specifically recognizing the at least three lipid metabolite biomarkers and the at least one additional cardiac biomarker, respectively. Suitable devices may be spectrometric devices such as mass spectrometry, NMR devices or devices for carrying out chemical or biological assays for the biomarkers. Suitable agents may be compounds which specifically detect the biomarkers. Detection as used herein may be a two-step process, i.e. the compound may first bind specifically to the biomarker to be detected and subsequently generate a detectable signal, e.g., fluorescent signals, chemiluminescent signals, radioactive signals and the like. For the generation of the detectable signal further compounds may be required which are all comprised by the term “means for determination of the at least one biomarker”.

In general, the present invention contemplates the use of at least three lipid metabolite biomarkers and the at least one additional cardiac biomarker as referred to herein in connection with the method of diagnosing a cardiac disease, and optionally of BNP or NT-proBNP, in a sample of a subject for diagnosing a cardiac disease. Preferred combinations of biomarkers and subforms of a cardiac disease to be diagnosed are disclosed elsewhere herein.

The present invention also relates to a kit for carrying out the method of the present invention, said kit comprising detection agents for each of the biomarkers of the at least three lipid metabolite biomarkers and the at least one additional cardiac biomarker as set forth in connection with the method of diagnosing a cardiac disease. In an embodiment, the kit may further comprise a detection agent for NT-proBNP or BNP.

The term “kit” as used herein refers to a collection of the aforementioned components, preferably, provided separately or within a single container. The detection agents may be provided in the kit of the invention in a “ready-to-use” liquid form or in dry form. The kit may further include controls, buffers, and/or reagents. The kit also comprises instructions for carrying out the method of the present invention, as well as information on the reference values. These instructions may be in the form of a manual or may be electronically accessible information. The latter information may be provided on a data storage medium or device such as an optical storage medium (e.g., a Compact Disc) or directly on a computer or data processing device.

Suitable detection agents for the biomarkers have been specified elsewhere herein in detail. For example, the detection agents may be antibodies or aptameres or other molecules which are capable of binding to the biomarkers specifically. This applies in particular if the biomarkers are protein biomarkers.

The kit of the invention can be, preferably, used for carrying out the method of the present invention, i.e. for diagnosing a cardiac disease as specified elsewhere herein in detail.

Method of Monitoring a Cardiac Disease Therapy

Based on the determination of the amounts of the at least three lipid metabolite biomarkers and the at least one additional cardiac biomarker as referred to above in the context of the method for diagnosing heart disease, it can be even assessed on an individual basis whether a treatment will be effective, or not.

The present invention thus relates to a method for monitoring a cardiac disease therapy in a subject, comprising:

(a) determining the amounts of the at least three lipid metabolite biomarkers and the at least one additional cardiac biomarker in a first sample of said subject and calculating a first score based on the determined amounts,

(b) determining the amounts of the at least three lipid metabolite biomarkers and the at least one additional cardiac biomarker in a second sample of said subject and calculating a second score based on the determined amounts; and

(c) comparing the first score to the second score, whereby a cardiac disease therapy in said subject is to be monitored.

In an embodiment of the aforementioned method, the method further comprises the determination of the amount of BNP (brain natriuretic peptide, also known as B-type natriuretic peptide) or, in particular, NT-proBNP (N-terminus of the prohormone brain natriuretic peptide) in the first and second sample.

Thus steps (a) to (c) may be as follows:

(a) determining the amount of the at least three lipid metabolite biomarkers and the at least one additional cardiac biomarker, and the amount of BNP or NT-proBNP in a first sample of said subject and calculating a first score based on the determined amounts,

(b) determining the amounts of the at least three lipid metabolite biomarkers and the at least one additional cardiac biomarker, and the amount of BNP or NT-proBNP in a second sample of said subject and calculating a second score based on the determined amounts; and

(c) comparing the first score to the second score, whereby a cardiac disease therapy in said subject is to be monitored.

The definitions given herein above in connection of the method for diagnosing a cardiac disease preferably apply to the method of monitoring a cardiac disease therapy as well.

The aforementioned method, preferably, is an in vitro method. Moreover, it may comprise steps in addition to those explicitly mentioned above. For example, further steps may relate to sample pre-treatments or evaluation of the results obtained by the method. Preferably, step (a), (b) and/or (c) may in total or in part be assisted by automation, e.g., by a suitable robotic and sensory equipment for the determination in steps (a) and (b), or a computer-implemented comparison in step (c).

The term “subject” has been defined above in connection with the method of diagnosing a cardiac disease. Preferably, the subject to be tested in accordance with the method for monitoring a cardiac disease therapy shall suffer from a cardiac disease. Moreover, the subject as set forth in connection with the aforementioned method shall be treated for a cardiac disease, and thus shall receive a cardiac disease therapy. Preferably, said subject does not suffer from acute decompensation.

The term “monitoring a cardiac disease therapy” as used herein in the context of the aforementioned method, preferably, relates to assessing whether a subject responds to said therapy, or not. Accordingly, it is assessed whether a subject benefits from said therapy, or not. Preferably, a decrease of the second score as compared to the first score shall be indicative for a subject who responds to a cardiac disease therapy. In contrast, an increase of the second score as compared to the first score shall be indicative for a subject who does not respond to a cardiac disease therapy. Preferably, by carrying out the aforementioned method decisions can be made whether a cardiac disease therapy in said subject shall be continued, stopped or amended.

Preferably, a subject responds to a cardiac disease therapy, if said therapy improves the condition of the subject with respect to a cardiac disease. Preferably, a subject does not respond to said therapy, if said therapy does not the improve the condition of the subject with respect to a cardiac disease. In this case, the therapy may put the subject at risk of adverse side effects without any significant benefit to said subject (thereby generating useless health care costs).

The term “cardiac disease therapy” as used herein, preferably, includes any therapy for the treatment of a cardiac disease therapy.

Preferably, the therapy is heart failure therapy. The term “heart failure therapy” as used herein, preferably, includes any therapy for the treatment of heart failure. Preferred therapies are drug-based therapies. Preferably, the therapy of heart failure is a therapy which comprises or consists of the administration of at least one drug selected from the group consisting of: ACE Inhibitors (ACEI), Beta Blockers, AT1-Inhibitors, Aldosteron Antagonists, Renin Antagonists, Diuretics, Ca-Sensitizer, Digitalis Glykosides, antiplatelet agents, and Vitamin-K-Antagonists.

Alternatively, the heart failure therapy may be therapy with assist devices such as ventricular assist devices.

The term “score” has been explained in connection with the method of diagnosing a cardiac disease. The explanation applies accordingly.

The term “sample” has been described elsewhere herein. The definition applies accordingly. In the context of the aforementioned method, the amount of the at least three lipid metabolite biomarkers and the at least one additional cardiac biomarker as referred to herein shall be determined in a first and in a second sample. Preferably, the first sample has been/is obtained before initiation of a cardiac disease therapy, or more preferably, after initiation of a cardiac disease therapy.

If the first sample has been obtained before initiation of a cardiac disease therapy, it is preferred that it has been obtained shortly before said initiation. Preferably, a sample is considered to have been obtained shortly before initiation of a cardiac disease therapy, if it has been obtained within less than one week, or, more preferably, within less than three days, or, most preferably, within less than one day before initiating a cardiac disease therapy.

The “second sample” is particularly understood as a sample which is obtained in order to reflect a change of the second score (i.e. the score in the second sample) to the first score (i.e. the score in the first sample). Thus, the second sample, preferably, shall have been obtained after the first sample. Of course, the second sample shall have been obtained after initiation of a cardiac disease therapy. It is to be understood that the second sample has been obtained not too early after the first sample in order to observe a sufficiently significant change of the score to allow for monitoring a cardiac disease therapy. Therefore, the second sample has been, preferably, obtained at least one week, or, more preferably, at least two weeks, or even more preferably, at least one month or two months, or, most preferably, at least three months after the first sample has been obtained. Also preferably, it is contemplated that the second sample has been obtained within a period of one week to three months after the first sample.

If the first sample has been obtained before initiation of a cardiac disease therapy, the second sample has been, preferably, obtained at least one week, or, more preferably, at least two weeks, or even more preferably, at least one month or two months, or, most preferably, at least three months after initiation of a cardiac disease therapy.

It shall be clear from the above that the determination of the amounts of the at least three lipid metabolite biomarkers and the at least one cardiac biomarker (and optionally BNP or NT-proBNP) in said first sample referred to in step (a) may take place several days or weeks before the determination of the amounts in said second sample referred to in step (b). Therefore the steps (a), (b) and (c) of the method for monitoring a cardiac disease need not be conduct one after the other in a limited time frame but may well be spread over a longer time period of several days, weeks or even months. Thus, it is to be understood that the aforementioned method allows for short-term, mid-term, and also for long-term monitoring depending on the interval between obtaining the two samples. Thus, the second sample may be obtained within a period of one day to two years or more after the first sample. In one preferred embodiment, the second sample has been obtained one day, or two days, in particular within a period of one to two days, after the first sample (which allows for short-term monitoring). In one another preferred embodiment, the second sample has been obtained one months, or two months, in particular within a period of one to two months, after the first sample (which allows for mid-term monitoring). In a further preferred embodiment, the second sample has been obtained six months, or twelve months, in particular within a period of six to twelve months or more, after the first sample (which allows for long-term monitoring).

It is also envisaged to assess the time course of the scores in samples from the subject to be monitored. Accordingly, the aforementioned method may comprise the additional step of determining the amounts of the at least three lipid metabolite biomarkers and the at least on cardiac biomarker in at least one further sample from said subject (thus, in a third sample, in a fourth sample, in a fifth sample etc.), calculating at least one further scores, and comparing the, thus, calculated score with the first and/or second score and/or any score that was calculated before said at least one further sample was obtained. For preferred time intervals for obtaining the samples, please see above.

Preferably, the assessment whether the subject responds to a cardiac disease therapy, or not, is based on the comparison of second score from the subject with the first score.

Preferably, a decrease and, more preferably, a significant decrease, and, most preferably, a statistically significant decrease of the second score as compared to the first score is indicative for a subject who responds to a cardiac disease therapy.

A significant decrease, preferably, is a decrease of a size which is considered to be significant for monitoring a cardiac disease. Particularly said decrease is considered statistically significant. The terms “significant” and “statistically significant” are known by the person skilled in the art. Thus, whether a decrease is significant or statistically significant can be determined without further ado by the person skilled in the art using various well known statistic evaluation tools. Preferred significant decreases of the score which are indicative for a subject who responds to a cardiac disease therapy are given herein below

Preferably, a decrease of the second score compared to the first score, preferably, of at least 5%, of at least 10%, more preferably of at least 20%, and, even more preferably, of at least 30%, and most preferably of at least 40% is considered to be significant and, thus, to be indicative for a subject who responds to a cardiac disease therapy. Also preferably, a decrease of the second score compared to the first score, preferably, of at least 0.05, of at least 0.10, more preferably of at least 0.15 and most preferably of at least 0.2 is considered to be significant and, thus, to be indicative for a subject who responds to a cardiac disease therapy.

As set forth above, an increase of the second score compared with the first score (or an, in particular, an essentially unchanged second score as compared with the first score) is indicative for a subject who does not respond to a cardiac disease therapy.

The following Examples shall illustrate the invention. They shall, however, not be construed as limiting the scope of the invention.

### EXAMPLES

### Example 1: Study Design

A multicentric study with three clinical centers and in total 843 subjects was conducted. The study comprised 194 male and female DCMP, 183 male and female ICMP and 210 male and female HFpEF patients as well as 256 male and female healthy controls in an age range from 35-75 and a BMI range from 20-35 kg/m². NYHA (New York Heart Association) scores of the patients ranged from I to III. Patients and controls were matched for age, gender and BMI. For all patients and controls, a blood sample was collected. Plasma was prepared by centrifugation, and samples were stored at −80° C. until measurements were performed.

Three subgroups of CHF (congestive heart failure) (DCMP, ICMP and HFpEF) were defined on the basis of echocardiography and hemodynamic criteria:

a) Subgroup DCMP: is hemodynamically defined as a systolic pump failure with cardic dilation (echocardiographic enhancement of the left ventricular end diastolic diameter >55 mm and a restricted left ventricular ejection fraction (LVEF) of <50%) without the presence of >50% stenosis

b) Subgroup ICMP: is hemodynamically defined as systolic pump failure due to a coronary insufficiency (>50% coronary stenosis and LVEF of <50%)

c) Subgroup heart failure with preserved ejection fraction (HFpEF): concentric heart hypertrophy (echocardiography: cardiac septum >12 mm and posterior myocardial wall >11 mm) and with a diastolic heart failure (non or mildly impaired pump function with LVEF of 50%) without a cardiac septum thickness >18 mm.

NYHA IV patients were excluded as well as patients suffering from apoplex, patients who had myocardial infarction within the last 4 months before testing, patients with altered medications within the last 4 weeks before testing as well as patients who suffered from acute or chronic inflammatory diseases and malignant tumours.

### Example 2: Determination of Metabolites for the “at Least Three Lipid Metabolite Biomarker” Panels Shown in Table 2

Significantly altered lipid metabolite biomarkers in CHF vs healthy respectively HFrEF vs healthy patients have been identified. Lipid metabolite biomarkers have been selected according to their accessibility with a one-shot LC-MS/MS measurement with a simplified preanalytical process (see analytical method description, below).

An LC-MS/MS method for the analysis of the at least three lipid metabolite biomarkers was established. This method is capable to analyze all of the biomarkers as listed in Table 1, where the biomarkers are characterized by the combination of retention time and multiple reaction monitoring (MRM) transitions, and further potential lipid metabolites.

Each biomarker of Table 1 may contain more than one analyte, whereby the analytes contained in the same biomarker have the same total number of carbon atoms and the same total number of double bonds.

TABLE 1
Lipid metabolite biomarkers used for composition of the “at least three lipid
metabolite biomarker” panels shown in Table 2 and their analytical characteristics.

| | | | | | Transition | |
|---|---|---|---|---|---|---|
| | | | | | (parent/ | Retention |
| | | | | | fragment) | Time |
| Biomarker | Analyte 1 | Analyte 2 | Analyte 3 | Group | (Da) | (min) |
| Cer(d16:1/24:0) | Cer(d16:1/24:0) | | | Ceramide | 622.6/236.2 | 2.83 |
| Cer(d17:1/24:0) | Cer(d17:1/24:0) | | | Ceramide | 636.6/250.2 | 2.95 |
| Cer(d18:1/23:0) | Cer(d18:1/23:0) | | | Ceramide | 636.5/264.2 | 2.95 |
| Cer(d18:1/24:1) | Cer(d18:1/24:1) | | | Ceramide | 648.6/264.2 | 2.83 |
| Cer(d18:2/24:0) | Cer(d18:2/24:0) | | | Ceramide | 648.6/262.2 | 2.9 |
| CE C18:0 | CE C18:0 | | | Cholesterylester | 670.7/369.3 | 4.72 |
| CE C18:2 | CE C18:2 | | | Cholesterylester | 666.7/369.3 | 4.47 |
| Glutamic acid | Glutamic acid | | | amino acid | 148.0/84.1 | 0.23 |
| PC4 | PC (C16:0 C18:2) | | | Phosphatidylcholine | 758.6/184.1 | 1.72 |
| PC8 | PC (C18:0 C18:2) | PC (C18:1 C18:1) | | Phosphatidylcholine | 786.6/184.1 | 1.88 |
| SM10 | SM(d18:1/18:0) | SM(d16:1/20:0) | | Sphingomyeline | 731.5/184.1 | 1.7 |
| SM18 | SM(d18:1/21:0) | SM(d16:1/23:0) | SM(d17:1/22:0) | Sphingomyeline | 773.5/184.1 | 1.97 |
| SM2 | SM(d18:1/14:0) | SM(d16:1/16:0) | | Sphingomyeline | 675.5/184.1 | 1.47 |
| SM21 | SM(d17:1/23:0) | SM(d18:1/22:0) | SM(d16:1/24:0) | Sphingomyeline | 787.5/184.1 | 2.05 |
| SM23 | SM(d18:1/23:1) | SM(d18:2/23:0) | SM(d17:1/24:1) | Sphingomyeline | 799.5/184.1 | 2.02 |
| SM24 | SM(d18:1/23:0) | SM(d17:1/24:0) | | Sphingomyeline | 801.5/184.1 | 2.16 |
| SM28 | SM(d18:1/24:0) | | | Sphingomyeline | 815.5/184.1 | 2.27 |
| SM29 | SM(d18:2/17:0) | | | Sphingomyeline | 715.5/184.1 | 1.57 |
| SM3 | SM(d17:1/16:0) | | | Sphingomyeline | 689.5/184.1 | 1.52 |
| SM5 | SM(d18:1/16:0) | SM(d16:1/18:0) | | Sphingomyeline | 703.5/184.1 | 1.57 |
| SM8 | SM(d18:2/18:1) | | | Sphingomyeline | 727.5/184.1 | 1.55 |
| SM9 | SM(d18:1/18:1) | SM(d18:2/18:0) | | Sphingomyeline | 729.5/184.1 | 1.62 |
| OSS2 | TAG C18:1 C18:0 C18:0 | | | Triacylglyceride | 906.9/605.4 | 4.87 |
| PPO1 | TAG C16:0 C16:0 C18:1 | | | Triacylglyceride | 850.8/577.5 | 4.64 |
| PPP | TAG C16:0 C16:0 C16:0 | | | Triacylglyceride | 824.8/551.5 | 4.61 |
| SOP2 | TAG C18:0 C18:1 C16:0 | | | Triacylglyceride | 878.9/577.6 | 4.76 |
| SPP1 | TAG C18:0 C16:0 C16:0 | | | Triacylglyceride | 852.9/579.6 | 4.74 |
| SSP2 | TAG C18:0 C18:0 C16:0 | | | Triacylglyceride | 880.9/579.6 | 4.85 |
| SSS | TAG C18:0 C18:0 C18:0 | | | Triacylglyceride | 908.9/607.6 | 4.96 |

In Table 2, the “at least three lipid metabolite biomarker” panels (for combination with “at least one cardiac biomarker” and optionally NT-proBNP) according to the present invention are listed. In column 1 the respective panel number is given, in column 2 the composition of each “at least three lipid metabolite biomarker” panel, and in column 3 the number of lipid metabolite biomarkers is given.

TABLE 2
“at least three lipid metabolite biomarker” panels for combination with “at
least one additional cardiac biomarker” and optionally BNP or NT-proBNP, in particular NT-proBNP

| Panel | | No. of |
|---|---|---|
| Number | Composition of the _{“}at least three lipid metabolite biomarker” panel | Biomarkers |
| 1 | SM23; OSS2; PC4 | 3 |
| 2 | OSS2; SM23; CE C18:2 | 3 |
| 3 | SOP2; OSS2; PC4; CE C18:2; SM18; SM28; SM24; SSP2; SM23 | 9 |
| 4 | OSS2; CE C18:2; SM23 | 3 |
| 5 | SOP2; OSS2; SM18; CE C18:2; SM24; SM28; Cer(d16:1/24:0); PC4 | 8 |
| 6 | SM18; OSS2; CE C18:2 | 3 |
| 7 | SM23; SOP2; CE C18:2 | 3 |
| 8 | SM23; SOP2; CE C18:2 | 3 |
| 9 | SOP2; SM24; SSP2; SM23; CE C18:2; SM28; SM18; PC4 | 8 |
| 10 | SM23; SOP2; SM28 | 3 |
| 11 | SM24; SOP2; CE C18:2; SM28; SSP2; SM18; OSS2; SM2; Cer(d16:1/24:0) | 9 |
| 12 | SM24; SOP2; CE C18:2 | 3 |
| 13 | OSS2; PC4; SM23 | 3 |
| 14 | OSS2; CE C18:2; SM3 | 3 |
| 15 | SM18; SM28; PC4; OSS2; SOP2; CE C18:2; PPO1; SM10 | 8 |
| 16 | CE C18:2; SOP2; SM18 | 3 |
| 17 | SM18; OSS2; SOP2; CE C18:2; PC4; SPP1; SM24; Cer(d16:1/24:0); SM28; SM21 | 10 |
| 18 | SM18; CE C18:2; OSS2 | 3 |
| 19 | SM23; SOP2; CE C18:2 | 3 |
| 20 | OSS2; SM23; CE C18:2 | 3 |
| 21 | CE C18:2; OSS2; SM23; SM24; SSP2 | 5 |
| 22 | OSS2; SM23; CE C18:2 | 3 |
| 23 | SM23; CE C18:2; OSS2; SSP2; SM24 | 5 |
| 24 | SM24; SSP2; OSS2 | 3 |
| 25 | SOP2; PC4; SM3 | 3 |
| 26 | OSS2; PC4; SM3 | 3 |
| 27 | SSP2; PPO1; SM18; CE C18:2; OSS2; SOP2; PC4; SM28 | 8 |
| 28 | SOP2; SM28; PC4 | 3 |
| 29 | Cer(d16:1/24:0); SM28; PC4; SM24; CE C18:2; SPP1; OSS2; SOP2 | 8 |
| 30 | OSS2; CE C18:2; SM24 | 3 |
| 31 | SOP2; SM23; PC4 | 3 |
| 32 | OSS2; CE C18:2; SM23 | 3 |
| 33 | SM24; SOP2; OSS2; CE C18:2; SSP2; PC4; SM28 | 7 |
| 34 | SOP2; SM24; PC4 | 3 |
| 35 | OSS2; CE C18:2; SOP2; SM18; SM24; SSP2; SM28; PC4 | 8 |
| 36 | OSS2; SM24; CE C18:2 | 3 |
| 37 | SM5; PPO1; SM23 | 3 |
| 38 | SM5; SOP2; SM24 | 3 |
| 39 | SSP2; CE C18:2; SM18; SM23; Cer(d16:1/24:0); PC4; SOP2; PPO1; SM28; SM24 | 10 |
| 40 | SM24; SOP2; PC4 | 3 |
| 41 | PC4; SM5; CE C18:2; SM2; PPO1; SOP2; SM28; Cer(d16:1/24:0); SM24 | 9 |
| 42 | SM24; CE C18:2; SOP2 | 3 |
| 43 | Cer(d18:1/24:1); SOP2; CE C18:2; SM18 | 4 |
| 44 | SM18; CE C18:2; SOP2 | 3 |
| 45 | SOP2; SM24; CE C18:2; SM18 | 4 |
| 46 | SM18; CE C18:2; SOP2 | 3 |
| 47 | SSP2; PC4; Cer(d18:1/24:1); SM28; SM24; SOP2; CE C18:2; SM18 | 8 |
| 48 | SM18; CE C18:2; SOP2 | 3 |
| 49 | SM28; CE C18:2; SOP2; SM18 | 4 |
| 50 | SM18; CE C18:2; SOP2 | 3 |
| 51 | SM23; CE C18:2; SOP2 | 3 |
| 52 | SM23; CE C18:2; SOP2 | 3 |
| 53 | SOP2; SM24; CE C18:2; SM18; PC4; SM28; PPO1; Cer(d16:1/24:0); OSS2; Cer(d18:1/24:1) | 10 |
| 54 | SM18; CE C18:2; SOP2 | 3 |
| 55 | SOP2; SM18; SM24; CE C18:2; Cer(d16:1/24:0); SM28; OSS2; PC4 | 8 |
| 56 | SM18; CE C18:2; SOP2 | 3 |
| 57 | SOP2; SM23; PC4 | 3 |
| 58 | SOP2; SSP2; SM5; SPP1; SM2; SM3; PC4; CE C18:2; Glutamic acid | 9 |
| 59 | SOP2; SM5; SM2 | 3 |
| 60 | SOP2; SM23; SSP2; SM24; CE C18:2; PC4; OSS2 | 7 |
| 61 | SOP2; SM23; CE C18:2 | 3 |
| 62 | SOP2; SSP2; SM2; CE C18:2; SM24; OSS2; SM18; PC4; SM28 | 9 |
| 63 | SOP2; SM18; CE C18:2 | 3 |
| 64 | SOP2; PC4; OSS2; Cer(d18:1/24:1); SM24; PC8; SM21; Cer(d17:1/24:0) | 8 |
| 65 | SOP2; PC4; SM24 | 3 |
| 66 | SOP2; SM18; PC4; SM21 | 4 |
| 67 | SOP2; SM18; PC4 | 3 |
| 68 | SOP2; PC4; SM28; SM18; Cer(d18:1/24:1); Cer(d18:2/24:0); SM9 | 7 |
| 69 | SOP2; PC4; SM18 | 3 |
| 70 | PC4; SOP2; SM18; SM24; OSS2; SPP1; SM21; Cer(d16:1/24:0); SM28; CE C18:2 | 10 |
| 71 | SM18; SOP2; PC4 | 3 |
| 72 | SOP2; PC4; SM5 | 3 |
| 73 | SOP2; SM3; PC4; SM21 | 4 |
| 74 | SOP2; SM3; PC4 | 3 |
| 75 | SOP2; SM18; PC4; SM28; OSS2; SSP2; CE C18:2 | 7 |
| 76 | SOP2; SM18; PC4 | 3 |
| 77 | SOP2; OSS2; SM18; SM24; PC4; CE C18:2; Cer(d16:1/24:0); SM28 | 8 |
| 78 | OSS2; SM18; CE C18:2 | 3 |
| 79 | SPP1; SOP2; SM5; PC4; SM3; SSP2 | 6 |
| 80 | SPP1; SM5; PC4 | 3 |
| 81 | SPP1; SM3; SM5; SSP2; PC4; Glutamic acid; CE C18:0; OSS2; SOP2 | 9 |
| 82 | SPP1; SM3; SM5 | 3 |
| 83 | SPP1; SOP2; SSP2; PC4; SM23; SM3; SM3; CE C18:2 | 8 |
| 84 | SPP1; SM23; SOP2 | 3 |
| 85 | SM3; SPP1; SSP2; SM5; SOP2; PC4 | 6 |
| 86 | SPP1; SM3; SM5 | 3 |
| 87 | SOP2; Cer(d18:1/24:1); PC8; PPO1; Cer(d18:1/23:0); PC4; SM24; SM28; SM9 | 9 |
| 88 | SOP2; PC4; SM24 | 3 |
| 89 | Cer(d18:1/23:0); SM24; SOP2; Cer(d18:1/24:1); PC4; PPO1; PCS; SSS; SM18 | 9 |
| 90 | SM24; SOP2; PC4 | 3 |
| 91 | PC4; SM28; SM24; Cer(d18:1/24:1); PC8; SOP2; SM9; PPO1; SM21; SSP2 | 10 |
| 92 | PC4; SM28; SOP2 | 3 |
| 93 | SM24; PC4; SM18; SOP2; SM21; SPP1; Cer(d18:1/23:0); SSP2 | 8 |
| 94 | SM24; SM18; SOP2 | 3 |
| 95 | SOP2; PC8; SPP1; PC4; SSP2; PPO1; SM5; SM18; Cer(d17:1/24:0) | 9 |
| 96 | SOP2; PC8; SM5 | 3 |
| 97 | SPP1; PC4; SM3; SOP2; SSP2; Cer(d18:1/23:0); SM24; SM5; Glutamic acid | 9 |
| 98 | SPP1; SM3; PC4 | 3 |
| 99 | SOP2; PC4; SPP1; PC8; SSP2; SM28; SM24; PPO1; SM18; Cer(d17:1/24:0) | 10 |
| 100 | SOP2; PC4; SM24 | 3 |
| 101 | PC4; SPP1; SM24; SSP2; SM3; SOP2; SM18; Glutamic acid; Cer(d17:1/24:0); Cer(d18:1/23:0) | 10 |
| 102 | SPP1; SM18; PC4 | 3 |
| 103 | CE C18:2; SSS; Cer(d17:1/24:0) | 3 |
| 104 | PC4; SOP2; CE C18:2 | 3 |
| 105 | CE C18:2; PC4; SM29; SOP2 | 4 |
| 106 | CE C18:2; PC8; SM29; SSP2 | 4 |
| 107 | CE C18:2; PC4; SM8; SSP2 | 4 |
| 108 | CE C18:2; PC8; SM8; SSP2 | 4 |
| 109 | CE C18:2; PC4; SM29; PPO1 | 4 |
| 110 | CE C18:2; PC8; SM29; PPO1 | 4 |
| 111 | CE C18:2; PC4; SM8; PPO1 | 4 |
| 112 | CE C18:2; PC8; SM8; PPO1 | 4 |
| 113 | CE C18:2; PC4; SM29; PPP | 4 |
| 114 | CE C18:2; PC8; SM29; PPP | 4 |
| 115 | CE C18:2; PC4; SM8; PPP | 4 |
| 116 | CE C18:2; PC8; SM29; SOP2 | 4 |
| 117 | CE C18:2; PC8; SM8; PPP | 4 |
| 118 | CE C18:2; PC4; SM8; SOP2 | 4 |
| 119 | CE C18:2; PC8; SM8; SOP2 | 4 |
| 120 | CE C18:2; PC4; SM29; SPP1 | 4 |
| 121 | CE C18:2; PC8; SM29; SPP1 | 4 |
| 122 | CE C18:2; PC4; SM8; SPP1 | 4 |
| 123 | CE C18:2; PC8; SM8; SPP1 | 4 |
| 124 | CE C18:2; PC4; SM29; SSP2 | 4 |
| 125 | PC4; SOP2; CE C18:2; SM18 | 4 |
| 126 | PC4; SOP2; CE C18:0; SM18 | 4 |
| 127 | PC4; SOP2; CE C18:2; SM21 | 4 |
| 128 | PC4; SOP2; CE C18:0; SM21 | 4 |
| 129 | PC4; SOP2; CE C18:2; SM23 | 4 |
| 130 | PC4; SOP2; CE C18:0; SM23 | 4 |
| 131 | PC4; SOP2; CE C18:2; SM24 | 4 |
| 132 | PC4; SOP2; CE C18:0; SM24 | 4 |
| 133 | SOP2; CE C18:0; SM18; PC4; SPP1 | 5 |
| 134 | SOP2; CE C18:2; SM21; PC4; SPP1 | 5 |
| 135 | SOP2; CE C18:0; SM21; PC4; SPP1 | 5 |
| 136 | SOP2; CE C18:2; SM23; PC4; SPP1 | 5 |
| 137 | SOP2; CE C18:0; SM23; PC4; SPP1 | 5 |
| 138 | SOP2; CE C18:2; SM24; PC4; SPP1 | 5 |
| 139 | SOP2; CE C18:0; SM24; PC4; SPP1 | 5 |
| 140 | SOP2; CE C18:2; SM18; PC4; SSP2 | 5 |
| 141 | SOP2; CE C18:0; SM18; PC4; SSP2 | 5 |
| 142 | SOP2; CE C18:2; SM21; PC4; SSP2 | 5 |
| 143 | SOP2; CE C18:0; SM21; PC4; SSP2 | 5 |
| 144 | SOP2; CE C18:2; SM23; PC4; SSP2 | 5 |
| 145 | SOP2; CE C18:0; SM23; PC4; SSP2 | 5 |
| 146 | SOP2; CE C18:2; SM24; PC4; SSP2 | 5 |
| 147 | SOP2; CE C18:0; SM24; PC4; SSP2 | 5 |
| 148 | SOP2; CE C18:2; SM18; PC4; PPO1 | 5 |
| 149 | SOP2; CE C18:0; SM18; PC4; PPO1 | 5 |
| 150 | SOP2; CE C18:2; SM21; PC4; PPO1 | 5 |
| 151 | SOP2; CE C18:0; SM21; PC4; PPO1 | 5 |
| 152 | SOP2; CE C18:2; SM23; PC4; PPO1 | 5 |
| 153 | SOP2; CE C18:0; SM23; PC4; PPO1 | 5 |
| 154 | SOP2; CE C18:2; SM24; PC4; PPO1 | 5 |
| 155 | SOP2; CE C18:0; SM24; PC4; PPO1 | 5 |
| 156 | SOP2; CE C18:2; SM18; PC4; PPP | 5 |
| 157 | SOP2; CE C18:0; SM18; PC4; PPP | 5 |
| 158 | SOP2; CE C18:2; SM21; PC4; PPP | 5 |
| 159 | SOP2; CE C18:0; SM21; PC4; PPP | 5 |
| 160 | SOP2; CE C18:2; SM23; PC4; PPP | 5 |
| 161 | SOP2; CE C18:0; SM23; PC4; PPP | 5 |
| 162 | SOP2; CE C18:2; SM24; PC4; PPP | 5 |
| 163 | SOP2; CE C18:0; SM24; PC4; PPP | 5 |
| 164 | SOP2; CE C18:2; SM18; PC4; SPP1 | 5 |
| 165 | PC4; PPP; CE C18:0; SM18 | 4 |
| 166 | PC4; SPP1; CE C18:2; SM21 | 4 |
| 167 | PC4; SSP2; CE C18:2; SM21 | 4 |
| 168 | PC4; PPO1; CE C18:2; SM21 | 4 |
| 169 | PC4; PPP; CE C18:2; SM21 | 4 |
| 170 | PC4; SPP1; CE C18:0; SM21 | 4 |
| 171 | PC4; SSP2; CE C18:0; SM21 | 4 |
| 172 | PC4; PPO1; CE C18:0; SM21 | 4 |
| 173 | PC4; SPP1; CE C18:2; SM18 | 4 |
| 174 | PC4; PPP; CE C18:0; SM21 | 4 |
| 175 | PC4; SPP1; CE C18:2; SM23 | 4 |
| 176 | PC4; SSP2; CE C18:2; SM23 | 4 |
| 177 | PC4; PPO1; CE C18:2; SM23 | 4 |
| 178 | PC4; PPP; CE C18:2; SM23 | 4 |
| 179 | PC4; SPP1; CE C18:0; SM23 | 4 |
| 180 | PC4; SSP2; CE C18:0; SM23 | 4 |
| 181 | PC4; PPO1; CE C18:0; SM23 | 4 |
| 182 | PC4; SSP2; CE C18:2; SM18 | 4 |
| 183 | PC4; PPP; CE C18:0; SM23 | 4 |
| 184 | PC4; SPP1; CE C18:2; SM24 | 4 |
| 185 | PC4; SSP2; CE C18:2; SM24 | 4 |
| 186 | PC4; PPO1; CE C18:2; SM24 | 4 |
| 187 | PC4; PPP; CE C18:2; SM24 | 4 |
| 188 | PC4; SPP1; CE C18:0; SM24 | 4 |
| 189 | PC4; SSP2; CE C18:0; SM24 | 4 |
| 190 | PC4; PPO1; CE C18:0; SM24 | 4 |
| 191 | PC4; PPO1; CE C18:2; SM18 | 4 |
| 192 | PC4; PPP; CE C18:0; SM24 | 4 |
| 193 | PC4; PPP; CE C18:2; SM18 | 4 |
| 194 | PC4; SPP1; CE C18:0; SM18 | 4 |
| 195 | PC4; SSP2; CE C18:0; SM18 | 4 |
| 196 | PC4; PPO1; CE C18:0; SM18 | 4 |
| 197 | PC4; SOP2; CE C18:2; SM28 | 4 |
| 198 | PC4; SOP2; CE C18:0; SM28 | 4 |
| 199 | SM18; SM24; SM28 | 3 |
| 200 | OSS2; SM23; CE C18:2; PC4 | 4 |
| 201 | CE C18:2; SM18; SSS | 3 |
| 202 | CE C18:2; PC8; SM18; SM2; SM24; SSS | 6 |
| 203 | CE C18:2; SM18; SM21; SM24; SSS | 5 |
| 204 | SM24; CE C18:2; SM2 | 3 |
| 205 | SM2; SSS; CE C18:2 | 3 |
| 206 | SM24; SSS; CE C18:2 | 3 |

Panels 1 to 206 were previously shown to allow for the diagnosis of heart failure (e.g. in combination with NT-proBNP). The results are shown in WO2016/034600. The International patent application claims the priorities of U.S. 62/044,367, EP 14183105.7 and U.S. 62/128,586. The International patent application as well as the three priority applications are herewith incorporated by reference in their entirety.

### Example 3: Analytical Method for the Determination of the “at Least Three Lipid Metabolite Biomarkers”

Human plasma samples were prepared and subjected to HPLC-MS/MS analysis as described in the following:

10 μl plasma were mixed with 1500 μl extraction solvent containing methanol/dichloromethane (in a ratio of 2:1, v/v) and 10 μl internal standard mixture in a 2 ml safelock microcentrifuge tube (Eppendorf, Germany). Lipid standards were purchased from Avanti Polar Lipids, CA, U.S.A., Larodan Fine Chemicals, Sweden, Sigma-Aldrich, MO, U.S.A., or Tokyo Chemical Industry, Japan.

Ultrapure water (Milli-Q water system, Millipore) and analytical grade chemicals were used for extraction, dilution or as LC solvents. Quality control and reference sample were prepared from commercially available human plasma (RECIPE Chemicals+Instruments GmbH). Delipidized plasma (Plasma, Human, Defibrinated, Delipidized, 2× Charcoal treated, Highly Purified; USBio) was used for the preparation of calibrators and blanks.

After thoroughly mixing at 20° C. for 5 min, the precipitated proteins were removed by centrifugation for 10 min. An aliquot of the liquid supernatant was transferred to an appropriate glass vial and stored at −20° C. until analysis by LC-MS/MS. This sample preparation method uses protein precipitation as the only purification step to capture all lipids of interest (PC, SM, Cer, CE, TAG), so that a more comprehensive and complex extraction of lipids (as described e.g. by Folch, or Bligh & Dyer) was not necessary. The HPLC-MS/MS systems consisted of an Agilent 1100 LC system (Agilent Technologies, Waldbronn, Germany) coupled to an ABSciex™ API 4000 triple quadrupole mass spectrometer (ABSCIEX, Toronto, Canada). HPLC analysis was performed at 55° C. on commercially available reversed phase separation columns with C18 stationary phases (Ascentis® Express C18 column (5 cm×2.1 mm, 2.7 μm, Phenomenex, Germany)).

Up to 5 μL of the crude extract were injected and separated by gradient elution using a mixture of solvents consisting of methanol, water, formic acid, 2-propanol and 2-methoxy-2-methylpropan:

Solvent A: 400 g methanol, 400 g water, 1 g formic acid

Solvent B: 400 g tert-butyl methyl ether (tBME), 200 g 2-propanol, 100 g methanol, 1 g formic acid

Mass spectrometry was carried out by electrospray ionization in positive ion mode using multiple-reaction-monitoring (MRM). The source parameters were: nebulizer gas, 50; heater gas, 60; curtain gas, 25; CAD gas, 4; ion spray voltage, 5500 V; temperature, 400° C.; pause between mass ranges, 5 ms; resolution Q1 and Q3, unit. To enhance the ionization efficiency in the electrospray process for some lipids (CE, TAG), ammonium formate buffer dissolved in methanol (Solvent C: 200 g methanol, 30 g 0.1M ammonium formate solution in water) was added post column during the elution time of CE and TAG. The method is intended to be compatible with e.g. the ABSciex™ 3200MD benchtop LC-MS/MS system.

Furthermore, as some highly abundant lipids are out of the dynamic range of the MS detector, MS parameters—like collision energy—were changed for said highly abundant lipids to get lower signal intensities due to lower fragmentation efficiencies.

Using electrospray isonization (ESI), PC 8 and several SMs with equal numbers of carbons and double bonds were detected together (as to say without separation of said species), since these isobaric species were not separated chromatographically. Chromatography was required to detect the low-concentrated ceramides and to separate phosphatidiyl cholines and sphingomyelins from each other, because the C13-impact of PC to SM or SM to PC disturbs the metabolite performance.

Positive and negative controls were prepared by lyophilization different amounts of commercially available plasma to meet the positive and negative cutoffs for all down regulated metabolites. For the triacylglycerides (TAG) the corresponding TAG was added into the plasma before the lyophilisation process but avoiding the protein to precipitate. The lyophilisated samples were stored in the freezertill sample preparation.

Quality control samples were prepared by extracting commercially available plasma with extraction solvent. Several aliquots of these extracts were stored in the freezer and used for the daily quality control of the instrument performance and sample preparation.

Plasma samples were analyzed in randomized analytical sequence design. Following comprehensive analytical validation steps, the resulting peak areas were divided by the peak areas of an internal standard with similar analytical behavior to reduce analytical variation. The resulting ratios were log 10-transformed to achieve normal distribution.

### Example 4: Data Analysis and Statistical Evaluation

For each lipid metabolite biomarker listed in Table 1, the direction of change in CHF patients relative to healthy control subjects was calculated by ANOVA (see Tables 1A and 1B). The direction ‘Up’ means that the levels of the biomarker are higher in CHF patients than in healthy control subjects, the direction ‘Down’ means that the levels of the biomarker are lower in CHF patients than in healthy control subjects. The direction of change was found to be the same for all CHF patients compared to healthy control subjects taken together [ANOVA model: CHF+CENTER+(GENDER+AGE+BMI)̂2], for HFpEF patients compared to healthy control subjects, and for HFrEF patients compared to healthy control subjects [CHF_SUBGROUP+CENTER+(GENDER+AGE+BMI)̂2].

TABLE 1A
Results of the ANOVA analysis as described above regarding the different lipid metabolite biomarkers from Table 1.

| | CHF_ALL | | | | HFpEF | | | | HFrEF | | | |
|---|---|---|---|---|---|---|---|---|---|---|---|---|
| Biomarker | Direction | p < 0.05 | Ratio | p-Value | Direction | p < 0.05 | Ratio | p-Value | Direction | p < 0.05 | Ratio | p-Value |
| Cer(d16:1/24:0) | Down | Yes | 0.76 | 3.08E−09 | Down | Yes | 0.84 | 1.74E−03 | Down | Yes | 0.72 | 2.72E−11 |
| Cer(d17:1/24:0) | Down | Yes | 0.78 | 1.88E−09 | Down | Yes | 0.82 | 7.38E−05 | Down | Yes | 0.76 | 4.17E−10 |
| Cer(d18:1/23:0) | Down | Yes | 0.86 | 2.22E−06 | Down | Yes | 0.87 | 1.62E−04 | Down | Yes | 0.86 | 6.23E−06 |
| Cer(d18:1/24:1) | Down | No | 0.98 | 5.77E−01 | Down | No | 0.97 | 4.07E−01 | Down | No | 0.99 | 7.62E−01 |
| Cer(d18:2/24:0) | Down | Yes | 0.86 | 4.78E−05 | Down | Yes | 0.90 | 1.24E−02 | Down | Yes | 0.85 | 1.53E−05 |
| CE C18:0 | Down | No | 0.94 | 1.55E−01 | Down | No | 0.99 | 8.34E−01 | Down | No | 0.92 | 5.65E−02 |
| CE C18:2 | Down | Yes | 0.87 | 1.21E−13 | Down | Yes | 0.92 | 1.31E−04 | Down | Yes | 0.85 | 2.72E−17 |
| Glutamic acid | Down | No | 0.93 | 1.31E−01 | Down | Yes | 0.89 | 3.62E−02 | Down | No | 0.96 | 3.62E−01 |
| OSS2 | Up | Yes | 1.75 | 2.67E−11 | Up | Yes | 1.54 | 2.03E−05 | Up | Yes | 1.87 | 2.13E−12 |
| PC4 | Down | Yes | 0.90 | 1.31E−08 | Down | Yes | 0.92 | 1.09E−04 | Down | Yes | 0.89 | 5.37E−09 |
| PC8 | Down | Yes | 0.92 | 6.89E−05 | Down | Yes | 0.93 | 4.44E−03 | Down | Yes | 0.92 | 6.56E−05 |
| PPO1 | Up | Yes | 1.49 | 1.29E−07 | Up | Yes | 1.35 | 1.16E−03 | Up | Yes | 1.57 | 1.81E−08 |
| PPP | Up | Yes | 1.61 | 1.16E−06 | Up | Yes | 1.46 | 1.39E−03 | Up | Yes | 1.70 | 4.38E−07 |
| SM10 | Down | Yes | 0.95 | 2.18E−02 | Down | No | 0.96 | 8.57E−02 | Down | Yes | 0.95 | 2.44E−02 |
| SM18 | Down | Yes | 0.79 | 1.41E−18 | Down | Yes | 0.84 | 8.68E−08 | Down | Yes | 0.76 | 1.55E−21 |
| SM2 | Down | Yes | 0.81 | 3.77E−13 | Down | Yes | 0.85 | 3.14E−06 | Down | Yes | 0.79 | 1.83E−14 |
| SM21 | Down | Yes | 0.84 | 4.51E−15 | Down | Yes | 0.89 | 1.22E−05 | Down | Yes | 0.81 | 2.54E−18 |
| SM23 | Down | Yes | 0.83 | 2.44E−15 | Down | Yes | 0.88 | 1.70E−06 | Down | Yes | 0.81 | 1.07E−17 |
| SM24 | Down | Yes | 0.81 | 2.42E−17 | Down | Yes | 0.86 | 5.47E−07 | Down | Yes | 0.78 | 1.92E−20 |
| SM28 | Down | Yes | 0.83 | 1.22E−13 | Down | Yes | 0.90 | 2.92E−04 | Down | Yes | 0.79 | 8.08E−18 |
| SM29 | Down | Yes | 0.86 | 4.18E−07 | Down | Yes | 0.88 | 3.22E−04 | Down | Yes | 0.85 | 3.49E−07 |
| SM3 | Down | Yes | 0.81 | 4.76E−14 | Down | Yes | 0.83 | 8.12E−08 | Down | Yes | 0.79 | 2.51E−14 |
| SM5 | Down | Yes | 0.89 | 1.94E−10 | Down | Yes | 0.90 | 2.20E−06 | Down | Yes | 0.88 | 3.05E−10 |
| SM8 | Down | Yes | 0.87 | 5.27E−07 | Down | Yes | 0.89 | 2.50E−04 | Down | Yes | 0.87 | 5.99E−07 |
| SM9 | Down | No | 0.97 | 2.21E−01 | Down | No | 0.98 | 4.67E−01 | Down | No | 0.97 | 1.89E−01 |
| SOP2 | Up | Yes | 1.70 | 7.93E−11 | Up | Yes | 1.50 | 3.73E−05 | Up | Yes | 1.81 | 6.21E−12 |
| SPP1 | Up | Yes | 1.75 | 3.54E−09 | Up | Yes | 1.59 | 5.96E−05 | Up | Yes | 1.85 | 1.32E−09 |
| SSP2 | Up | Yes | 1.64 | 2.51E−09 | Up | Yes | 1.50 | 6.41E−05 | Up | Yes | 1.73 | 7.37E−10 |
| SSS | Up | Yes | 1.27 | 1.11E−04 | Up | Yes | 1.22 | 1.06E−02 | Up | Yes | 1.31 | 6.26E−05 |

TABLE 1B
Results of the ANOVA analysis for CHF subgroups ICMP and DCMP as described
above regarding the different lipid metabolite biomarkers from Table 1.
Only the lipid metabolite biomarkers from Panel 1 in Table 2 are listed.

| | ICMP | | | | DCMP | | | |
|---|---|---|---|---|---|---|---|---|
| Biomarker | Direction | p < 0.05 | Ratio | p-Value | Direction | p < 0.05 | Ratio | p-Value |
| OSS2 | Up | Yes | 1.77 | 5.01E−08 | Up | Yes | 1.96 | 2.85E−11 |
| PC4 | Down | Yes | 0.86 | 1.52E−10 | Down | Yes | 0.92 | 9.52E−05 |
| SM23 | Down | Yes | 0.78 | 6.64E−19 | Down | Yes | 0.84 | 3.67E−10 |

As comparison for the combination of the lipid metabolite biomarkers of Panel 1 with NT-proBNP and the additional cardic biomarkers HDL cholesterol and LDL cholesterol as mentioned in Example 8, the data of the study described in Example 1 were utilized for the evaluation of the diagnostic power for the classification of CHF subgroups compared with controls in combination with the peptide NT-proBNP. CHF patients were subdivided based on CHF subtype [HFpEF, DCMP, ICMP, or alternatively the joined DCMP+ICMP group named HFrEF (heart failure with reduced ejection fraction)] and a measure for the severety of the disease. Two different measures for the severety of the disease were used: NYHA class and LVEF. To this end, CHF patients were subdivided into those having no or only mild symptoms (NYHA class I and early stages of NYHA class II; sometimes referred to as ‘asymptomatic’) and those having more severe symptoms (NYHA classes II and III, sometimes referred to as ‘symptomatic’). Alternatively, CHF patient were subdivided into those with severely reduced LVEF (LVEF <35%) and those with mildly reduced or non-reduced LVEV (LVEF≥35). The latter subgroup comprises HFrEF patient with 35% LVEF <50% as well as all HFpEF patients (LVEF 50%).

A total set of about 850 samples was split into a training or identification set (also referred to as “ID Data” set; about 66% of the samples) and a testing set (also referred to as “VD Data” set; about 33% of the samples). The split was done group-wise, with groups being defined by the combination of gender, type of heart failure and NYHA levels.

The total sample numbers and compositions were as follows:

| ID Data Set: | VD Data Set: |
|---|---|
| CHF: 374 samples | CHF: 205 samples |
| CHF, NYHA I-I/II: 169 samples | CHF, NYHA I-I/II: 93 samples |
| CHF, NYHA II-III: 205 samples | CHF, NYHA II-III: 112 samples |
| CHF, LVEF ≥ 35%: 275 samples* | CHF, LVEF ≥ 35: 145 samples |
| HFpEF: 134 samples | HFpEF: 74 samples |
| ICMP: 118 samples | ICMP: 65 samples |
| ICMP, NYHA I-I/II: 44 samples | ICMP, NYHA I-I/II: 24 samples |
| ICMP, NYHA II-III: 74 samples | ICMP, NYHA II-III: 41 samples |
| DCMP: 122 samples | DCMP: 66 samples |
| DCMP, NYHA I-I/II: 46 samples | DCMP, NYHA I-I/II: 25 samples |
| DCMP, NYHA II-III: 76 samples | DCMP, NYHA II-III: 41 samples |
| HFrEF: 240 samples | HFrEF: 131 samples |
| HFrEF, NYHA I-I/II: 90 samples | HFrEF, NYHA I-I/II: 49 samples |
| HFrEF, NYHA II-III: 150 samples | HFrEF, NYHA II-III: 82 samples |
| HFrEF, 35% ≤ LVEF < 50%: 141 samples* | HFrEF, 35% ≤ LVEF < 50%: 71 samples |
| HFrEF, LVEF < 35%: 98 samples* | HFrEF, LVEF < 35%: 60 samples |
| Controls: 167 samples | Controls: 87 samples** |

*One CHF patient with missing LVEF measurement was not included in these subgroups
**3 controls were subsequently excluded due to HF-related medication; re-analysis showed no significant changes with regard to the diagnostic performance of the “at least three lipid metabolite biomarker” panels

### Example 5

Prediction probabilities for each patient were calculated with a logistic regression model fitted using the elastic net algorithm as implemented in the R package glmnet (Zou, H. and Hastie, T., 2003: Regression shrinkage and selection via the elastic net, with applications to microarrays. Journal of the Royal Statistical Society: Series B, 67, 301-320; Friedman, J., Hastie, T., and Tibshirani, R, 2010: Regularization Paths for Generalized Linear Models via Coordnate Descent. J. Stat. Softw. 33) based on the “at least lipid metabolite biomarker” panel 1 as well as based on the “at least lipid metabolite biomarker” panel 1+NT-proBNP. The fitting was performed on the data from the ID cohort. The L1 and the L2 penalties were given equal weight. Log-transformed peak area ratios were centered and scaled to unit variance before the anaysis. The prediction probability was calculated using the formula

$$p = \frac{1}{1 + {e}^{- ( {w}_{0} + {Σ}_{i = 1}^{n} {w}_{i} {\hat{x}}_{i} )}} ,$$

with the feature \hat{x}ᵢ being

$${\hat{x}}_{i} = \frac{{x}_{i} - {m}_{i}}{{s}_{i}}$$

wherein xᵢ are the log-transformed peak area ratios (or concentrations, e.g. of NT-proBNP, if taken into account) and mᵢ, sᵢ are feature specific scaling factors and wᵢ are the coefficients of the model [w₀, intercept; w₁, coefficient for NT-proBNP (where applicable); w₂ . . . wₙ, coefficients for the features; n, number of lipid metabolite biomarkers+NT-proBNP, if determined or taken into account].

For example the coefficients for Panel 1+NT-proBNP are: WO=0.7602899 (Intercept), w₁=1.5745605 (NT-proBNP), w₂=−0.7158906 (SM23), w₃=0.6762354 (OSS2), w₄=−0.4594183 (PC4), n=4. The respective scaling factors are m1=2.2023800, m2=−0.5379916, m3=−2.1316954, m4=0.8736775 and s1=0.6364517, s2=0.1159420, s3=0.3863649, s4=0.0834807.

A sample with a prediction probability larger than the cutoff of the respective panel is said to be tested positive, and a sample with a prediction probability smaller than or equal to the cutoff is said to be tested negative. The sensitivity is the fraction of HF patients in the respective subgroup that are tested positive, while the specificity is the fraction of healthy control subjects that are tested negative.

The cutoff values can e.g. be determined to maximize the Youden index for the detection of the respective disease state.

Alternatively, the cutoff values can be determined to achieve e.g. the same specificity for the detection of the respective disease state in the ID dataset when using the“at least three lipid metabolite biomarker” panel as when using e.g. NT-proBNP alone (“fixed sensitivity”).

### Example 6: Validation of Performance in Testing Test

The performance of a “at least three lipid metabolite biomarker” panel identified in the training data when applied to the validation data was assessed by the Area Under the Curve (AUC) of a receiver operating curve, which was modelled with the binormal model.

### Example 7: Determination of Biomarkers for the “at Least One Additional Cardiac Biomarker”

In addition to at least three lipid metabolite biomarkers e.g. as described above, at least one additional cardiac biomarker described in the following is determined from a biological sample, e.g. blood plasma or blood serum sample.

(1) General Lipid Cardiac Biomarkers: Total Cholesterol, HDL Cholesterol, Triglycerides, LDLCholesterol, the Ratio of Total Cholesterol to HDL-Cholesterol, Non-HDL Cholesterol.

Total Cholesterol is determined by using commercially available tests, e.g. by a enzymatically based test using in addition a colorimetric method standardized to NIST (National Institute of Standards and Technology).

HDL cholesterol is measured by using commercially available tests, e.g. by separating from other lipoprotein fractions using e.g. either ultracentrifugation or chemical precipitation of other lipoprotein fractions with divalent ions such as Mg²⁺, within the resulting “HDL fraction” the lipoprotein-cholesterol/cholesterylester complex will be destroyed to allow the release of cholesterol and cholesterylesters. The cholesterylesters will be cleaved enzymatically with cholesteryl esterase to yield cholesterol which will be oxidised together with the already present cholesterol via cholesteroloxidase and the hereby produced H₂O₂ will be determined in an indicator reaction. HDL cholesterol is alternatively measured by an automated homogeneous analytical methods in which lipoproteins containing apo B are blocked using antibodies to apo B, then a colorimetric enzyme reaction measures cholesterol in the non-blocked HDL particles. HDL cholesterol is alternatively measured by HPLC or a turbidimetric method.

Triglycerides are measured by commercially availabale tests, e.g. determined enzymatically by first contacting the sample with lipase under conditions and for a time sufficient to allow conversion into glycerol and free fatty acids; then the sample comprising the glycerol is contacted with glycerokinase under conditions and for a time sufficient to allow conversion into glycerol-3-phosphate. Subsequently the sample comprising the glycerol-3-phosphate is contacted with glycerophosphate oxidase under conditions and for a time sufficient to allow conversion into dihydroxyacetone phosphate and H₂O₂; finally the amount of H₂O₂ is enzymatically or chemically determined.

LDL-cholesterol is determined by using commercially available tests or e.g. determined by using the Friedewald equation (Warnick G R, Knopp R H, Fitzpatrick V, Branson L (January 1990). “Estimating low-density lipoprotein cholesterol by the Friedewald equation is adequate for classifying patients on the basis of nationally recommended cutpoints”. Clinical Chemistry 36 (1): 15-9.PMID 2297909), by which the amount of LDL cholesterol is determined by subtraction of other cholesterol sources from total cholesterol. Friedewald equation: LDL cholesterol is approximately equal total cholesterol minus HDL-cholesterol concentrations minus triacylglycerols. The concentration of triacylglycerols is multiplied by a factor which is 0.20 if the quantities are measured in mg/dl and 0.45 if in mmol/l.

Non-HDL cholesterol is determined by subtracting HDL cholesterol from total cholesterol.

The ratio of total cholesterol to HDL-cholesterol is determined by determining total cholesterol as described above, by determining HDL-cholesterol as described above and by determining the ratio thereof.

(2) Lipoprotein subfraction biomarkers: LDL particles (herein also referred to as total LDL particles), small LDL particles, medium LDL particles and large HDL particles—Quantification and particle count of lipoprotein subfractions The lipoprotein subfraction biomarkers can be determined by ion mobility analysis. How to carry out such a ion mobility analysis is e.g. described in the publication Caulfield et al. (Clinical Chemistry August 2008 vol. 54 no. 8 1307-1316) which herewith is incorporated by reference with respect to the entire disclosure content. Ion mobility analysis preferably uses gas-phase electrophoresis to separate lipoproteins on the basis of size. This test is also commercially available from Quest Diagnostis Incorporated (Ion Mobility 91604(X).

(3) Apolipoprotein Biomarkers: Apolipoprotein B (ApoB), Lipoprotein (a)

ApoB is determined by an commercially available test being based on an immunoturbidimetric procedure that measures incre asing sample turbidity caused by the formation of insoluble immune complexes when antibody to ApoB is added to the sample. A sample containing ApoB is incubated with a buffer, and a sample blank determination is performed prior to the addition of ApoB antibody. In the presence of an appropriate antibody in excess, the ApoB concentration is measured as a function of turbidity

Lipoprotein (a) is determined by an commercially available test being based on a latex enhanced immunoturbidimetric method (Diazyme). Lp(a) in the sample binds to the specific antiLp(a) antibody, which is coated on latex particles, and causes agglutination. The degree of the turbidity caused by agglutination can be measured optically and is proportional to the amount of Lp(a) in the sample.

(4) Inflammation Biomarkers: HS CRP, Lp-PLA2 (PLAC):

hsCRPis determined by an commercially available test, with e.g. the Diazyme High Sensitive CReactive Protein (hsCRP) assay, which is based on a latex-enhanced turbidimetric immunoassay method. When an antigen-antibody reaction occurs between hs CRP in a sample and antiCRP antibody which is coated on latex particles, agglutination results. This agglutination is detected as an absorbance change (570 nm), with the magnitude of the change being proportional to the quantity of hs CRP in the sample. The actual concentration is then determined by interpolation from a calibration curve prepared from calibrators of known concentration.

Lp-PLA2 (PLAC) is determined by an commercially available test being a turbidimetric immunoassay for the quantitative determination of Lp-PLA2 (lipoprotein-associated phospholipase A2) in human plasma.

For each cardiac biomarker listed in Table 3, the direction of change in patients with cardiovascular disease relative to healthy control is listed (Table 3). This can also be calculated by ANOVA.

TABLE 3
Directions of change of the cardiac biomarkers indicative
for cardiovascular diseases or heart failure

| Cardiac biomarker | Direction vs. controls |
|---|---|
| Total cholesterol | up |
| HDL cholesterol | down |
| Triacylglycerols | up |
| LDL-cholesterol | up |
| non-HDL cholesterol | up |
| ratio of total cholesterol to HDL-cholesterol | up |
| LDL particle number | up |
| small LDL particles | up |
| medium LDL particles | up |
| large HDL particles | down |
| ApoB | up |
| Lp(a) | up |
| hsCRP | up |
| Lp-PLA2 (PLAC) | up |

One or more of the above cardiac biomarkers are combined with any of the Panels 1 to 206 above (including or excluding NTP-proBNP). Combination means that values for the additional cardiac biomarker and the respective lipid metabolite biomarkers and, if taken into account, NT-proBNP are used as features of the classification model (e.g. Elastic Net). If several cardiac biomarkers are used, each biomarker is added as additional feature to the classification model. At least one of Panels 1 to 206 and one or more additional cardiac biomarkers and, optionally, NT-proBNP are used. The model is designed for an improved diagnosis of CHF including its subforms (e.g. HFrEF, HFpEF) and/or any cardiovascular disease including peripheral artery disease, coronary artery disease, atherosclerosis, cardiomyopathy, pulmonary heart disease, and other cardiac diseases and the model is fit in analogy as described above (see e.g. Example 5).

### Example 8: Performance of Panel 1 Combined with NT-proBNP, HDL, and LDL

The concentrations of each of HDL cholesterol (HDLchol) and LDL cholesterol (LDLchol) were determined by respective commercially availiable assays in the respective samples referred to above. HDL cholesterol concentrations were determined turbidimetric using the “Direct HDL Cholesterin für Advia” Kit from Siemens Healthcare GmbH on an Advia automated system. The Adiva automated system fom Siemens Healthcare GmbH also measured the parameters additionally necessary for the calculation of LDL cholesterol via the Friedewald equation using the respective kits from Siemens for the Advia automated system. These parameters are “total cholesterol” and “triglycerides”. Subsequently LDL cholesterol concentrations were calculated from the Friedewald equation.

The lipid metabolite biomarkers of the “at least three lipid metabolite biomarkers” of Panel 1+NT-pro-BNP were used as one classification model, that was then extended by the values for HDL cholesterol as well as LDL cholesterol into a second classification model in order to compare the diagnostic performance with and without consideration of HDLchol and LDLchol. These two classification models were fit on the “ID Data” in the same way as described in Example 5, and performance estimates (Area under the curve (AUC) values of receiver operating characteristic (ROC) analysis) were calculated on the data set referred to as “VD data” above for different CHF subgroups (see Table 4).

Several subgroups benefitted from the use of Panel 1 and NT-proBNP and both HDL cholesterol and LDL cholesterol (Abbreviated as 1+NT+HDLchol+LDLchol) compared to Panel 1 and NT-proBNP alone (Abbreviated as 1+NT). The respective performance values are shown in Table 4.

Since HDLchol and/or LDLchol values were missing for some subjects, only patients with complete values for both HDLchol and LDLchol were used to fit and test the model.

For comparability, the subjects lacking data for HDLchol and/or LDLchol were also excluded for calculation of the 1+NT data, so that the same set of subjects was used for all calculations in Table 4. The cut-off was determined on the “ID Data” with a “fixed sensitivity” (i.e. so that the marker panel had the same sensitivity as NT-proBNP for the comparison of HFrEF vs controls). The resulting cut-off was 0.74997 when using HDLchol and LDLchol and 0.72131 when not.

TABLE 4
Performance of Panel 1 + NT-proBNP (abbreviated 1 + NT) has been compared to the performance
of Panel 1 + NT-proBNP + HDL cholesterol (abbreviated HDLchol) + LDL cholesterol (abbreviated
LDLchol. Area under the curve (AUC) values of receiver operating characteristic (ROC) analysis were calculated
for different CHF subgroups. Calculations were carried out on the dataset referred to as “VD data”
above, with the exception of subjects for which HDLchol or LDLchol data was missing (such subjects were excluded).

| | UsedPanel | Used | Used | NT-proBNP | | | |
|---|---|---|---|---|---|---|---|
| | AUC | Panel | Panel | AUC | NT-proBNP | NT-proBNP | |
| Subgroup | estimate | Sensitivity | Specificity | Estimate | Sensitivity | Specificity | Used Panel |
| CHF ASYMP | 0.8713 | 0.5000 | 0.9655 | 0.8078 | 0.5698 | 0.8736 | 1 + NT |
| CHF ASYMP | 0.8743 | 0.5000 | 0.9885 | 0.8078 | 0.5698 | 0.8736 | 1 + NT + HDLchol + LDLchol |
| DCM ASYMP | 0.8790 | 0.5000 | 0.9655 | 0.8599 | 0.6667 | 0.8736 | 1 + NT |
| DCM ASYMP | 0.8799 | 0.5000 | 0.9885 | 0.8599 | 0.6667 | 0.8736 | 1 + NT + HDLchol + LDLchol |
| HFpEF | 0.8091 | 0.2462 | 0.9655 | 0.7059 | 0.3538 | 0.8736 | 1 + NT |
| HFpEF | 0.8132 | 0.2308 | 0.9885 | 0.7059 | 0.3538 | 0.8736 | 1 + NT + HDLchol + LDLchol |
| HFpEF ASYMP | 0.7850 | 0.2105 | 0.9655 | 0.6569 | 0.2632 | 0.8736 | 1 + NT |
| HFpEF ASYMP | 0.7896 | 0.2105 | 0.9885 | 0.6569 | 0.2632 | 0.8736 | 1 + NT + HDLchol + LDLchol |
| HFpEF SYMP | 0.8401 | 0.2963 | 0.9655 | 0.7675 | 0.4815 | 0.8736 | 1 + NT |
| HFpEF SYMP | 0.8435 | 0.2593 | 0.9885 | 0.7675 | 0.4815 | 0.8736 | 1 + NT + HDLchol + LDLchol |
| HFrEF ASYMP | 0.9471 | 0.7292 | 0.9655 | 0.9191 | 0.8125 | 0.8736 | 1 + NT |
| HFrEF ASYMP | 0.9474 | 0.7292 | 0.9885 | 0.9191 | 0.8125 | 0.8736 | 1 + NT + HDLchol + LDLchol |

### Example 9: Performance of Panel 1 Combined with NT-proBNP and HDL

The very same approach as described in Example 8 was applied with a classification model consisting of the “at least three lipid metabolite biomarkers” of Panel 1+ and NT-pro-BNP and HDL cholesterol (Panel: “1+NT+HDLchol”). The resulting cut-off for this panel (determined on the “ID Data” with a “fixed sensitivity” as described in Example 8, above) was 0.748164136. A comparison of all three panels, “1+NT”, “1+NT+HDLchol+LDLchol”, and “1+NT+HDLchol”, is shown in Table 5, below.

TABLE 5
Performance of Panel 1 + NT-proBNP (abbreviated 1 + NT) has been compared to the performance
of Panel 1 + NT-proBNP + HDL cholesterol (abbreviated HDLchol) + LDL cholesterol (abbreviated
LDLchol), and to Panel 1 + NT-proBNP + HDL cholesterol (abbreviated HDLchol). Area under the
curve (AUC) values of receiver operating characteristic (ROC) analysis were calculated for different CHF
subgroups. Calculations were carried out on the dataset referred to as “VD data” above, with the
exception of subjects for which HDLchol or LDLchol data was missing (such subjects were excluded).

| | Used | | | | | | |
|---|---|---|---|---|---|---|---|
| | Panel | Used | Used | NT-proBNP | | | |
| | AUC | Panel | Panel | AUC | NT-proBNP | NT-proBNP | |
| Subgroup | Estimate | Sensitivity | Specificity | Estimate | Sensitivity | Specificity | Used Panel |
| CHF ASYMP | 0.8713 | 0.5000 | 0.9655 | 0.8078 | 0.5698 | 0.8736 | 1 + NT |
| CHF ASYMP | 0.8743 | 0.5000 | 0.9885 | 0.8078 | 0.5698 | 0.8736 | 1 + NT + HDLchol + LDLchol |
| CHF ASYMP | 0.8745 | 0.5000 | 0.9885 | 0.8078 | 0.5698 | 0.8736 | 1 + NT + HDLchol |
| DCM ASYMP | 0.8790 | 0.5000 | 0.9655 | 0.8599 | 0.6667 | 0.8736 | 1 + NT |
| DCM ASYMP | 0.8799 | 0.5000 | 0.9885 | 0.8599 | 0.6667 | 0.8736 | 1 + NT + HDLchol + LDLchol |
| DCM ASYMP | 0.8801 | 0.5000 | 0.9885 | 0.8599 | 0.6667 | 0.8736 | 1 + NT + HDLchol |
| HFpEF | 0.8091 | 0.2462 | 0.9655 | 0.7059 | 0.3538 | 0.8736 | 1 + NT |
| HFpEF | 0.8132 | 0.2308 | 0.9885 | 0.7059 | 0.3538 | 0.8736 | 1 + NT + HDLchol + LDLchol |
| HFpEF | 0.8159 | 0.2154 | 0.9885 | 0.7059 | 0.3538 | 0.8736 | 1 + NT + HDLchol |
| HFpEF ASYMP | 0.7850 | 0.2105 | 0.9655 | 0.6569 | 0.2632 | 0.8736 | 1 + NT |
| HFpEF ASYMP | 0.7896 | 0.2105 | 0.9885 | 0.6569 | 0.2632 | 0.8736 | 1 + NT + HDLchol + LDLchol |
| HFpEF ASYMP | 0.7938 | 0.2105 | 0.9885 | 0.6569 | 0.2632 | 0.8736 | 1 + NT + HDLchol |
| HFpEF SYMP | 0.8401 | 0.2963 | 0.9655 | 0.7675 | 0.4815 | 0.8736 | 1 + NT |
| HFpEF SYMP | 0.8435 | 0.2593 | 0.9885 | 0.7675 | 0.4815 | 0.8736 | 1 + NT + HDLchol + LDLchol |
| HFpEF SYMP | 0.8442 | 0.2222 | 0.9885 | 0.7675 | 0.4815 | 0.8736 | 1 + NT + HDLchol |
| HFrEF ASYMP | 0.9471 | 0.7292 | 0.9655 | 0.9191 | 0.8125 | 0.8736 | 1 + NT |
| HFrEF ASYMP | 0.9474 | 0.7292 | 0.9885 | 0.9191 | 0.8125 | 0.8736 | 1 + NT + HDLchol + LDLchol |
| HFrEF ASYMP | 0.9467 | 0.7292 | 0.9885 | 0.9191 | 0.8125 | 0.8736 | 1 + NT + HDLchol |

1. A method for diagnosing cardiac disease comprising the steps of:
a. determining in a sample of a subject the amounts of at least three lipid metabolite biomarkers and of at least one additional cardiac biomarker, and
b. comparing the amounts as determined in step a. to a reference, whereby cardiac disease is to be diagnosed.

2. The method of claim 1, wherein said at least three lipid metabolite biomarkers are:
i. at least one triacylglyceride biomarker, at least one cholesterylester biomarker, and at least one phosphatidylcholine biomarker;
ii. at least one triacylglyceride biomarker, at least one phosphatidylcholine biomarker, and at least one sphingomyelin biomarker;
iii. at least one triacylglyceride biomarker, at least one cholesterylester biomarker, and at least one sphingomyelin biomarker;
iv. at least one phosphatidylcholine biomarker, at least one cholesterylester biomarker, and at least one sphingomyelin biomarker;
v. Cholesterylester C18:2, SSS and Cer(d17:1/24:0);
vi. at least two sphingomyelin biomarkers selected from the group consisting of SM2, SM3, SM5, SM18, SM23, SM24, and SM28, and at least one triacylglyceride biomarker selected from the group consisting of SOP2, SPP1 and PPO1, (or alternatively at least one triacylglyceride biomarker selected from the group consisting of SOP2, SPP1 and PPP);
vii. at least two triacylglyceride biomarkers selected from the group consisting of OSS2, SOP2, SPP1 and SSP2, and at least one sphingomyelin biomarker selected from the group consisting of SM23 and SM24;
viii. SM18, SM24 and SM28; or
ix. the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, or 206 of Table 2.

3. The method of claim 2, wherein the at least one triacylglyceride biomarker in i., ii., and iii. is selected from the group consisting of SOP2, OSS2, SPP1, SSP2, PPO1 and PPP, the at least one cholesterylester biomarker in i., iii., and iv. is selected from the group consisting of cholesterylester C18:2 and cholesterylester C18:0, the at least one phosphatidylcholine biomarker in i., ii. and iv. is selected from the group consisting of PC4 and PC8, and the at least one sphingomyelin biomarker in ii., iii., and iv. is selected from the group consisting of SM18, SM24, SM23, SM21, SM28, SM5, SM3, SM29 and SM8.

4. The method of any one of claims 1 to 3, wherein the at least one additional cardiac marker is selected from the group consisting of at least one general lipid cardiac biomarker, at least one lipoprotein subfraction biomarker, at least one apolipoprotein biomarker and at least one inflammation biomarker.

5. The method of claim 4, wherein the at least one general lipid cardiac biomarker is selected from the group consisting of total cholesterol, HDL-cholesterol (High Density Lipoprotein Cholesterol), triglycerides, LDL-cholesterol (High Density Lipoprotein Cholesterol), the ratio of total cholesterol to HDL-cholesterol, and non-HDL cholesterol, preferably wherein the amount(s) of HDL cholesterol and/or LDL cholesterol is (are) determined, more preferably, wherein the amount of HDL cholesterol is determined as additional cardiac biomarker.

6. The method of claims 4 and 5, wherein the at least one lipoprotein subfraction biomarker is selected from LDL particles, small LDL particles, medium LDL particles and large HDL particles.

7. The method of any one claims 4 to 6, wherein the at least one apolipoprotein biomarker is selected from apolipoprotein B and lipoprotein (a).

8. The method of any one of claims 4 to 7, wherein the at least one inflammation biomarker is selected from C-reactive protein (CRP), in particular high sensitivity C-reactive protein (hsCRP) and Lipoprotein-associated phospholipase A2 (Lp-PLA2).

9. The method of any one of claims 4 to 8, wherein the amounts of total cholesterol, HDL-cholesterol (High Density Lipoprotein Cholesterol), triglycerides, LDL-cholesterol (High Density Lipoprotein Cholesterol), non-HDL cholesterol, LDL particles, small LDL particles, medium LDL particles, large HDL particles, hsCRP, Lp-PLA2, apolipoprotein B and lipoprotein(a) are determined as cardiac markers.

10. The method of claim 8, further comprising the determination of the ratio of total cholesterol to HDL-cholesterol.

11. The method of any one of claims 1 to 10, wherein the cardiac disease is selected from peripheral artery disease, coronary artery disease, atherosclerosis, cardiomyopathy, heart failure, and pulmonary heart disease.

12. The method of claim 11, wherein the cardiac disease is coronary artery disease.

13. The method of claim 11, wherein the cardiac disease is heart failure.

14. The method of claims 11 and 13, wherein the heart failure is heart failure with reduced left ventricular ejection fraction (HFrEF).

15. The method any one of the preceding claims, wherein at least the amounts of the biomarkers of i., ii., iii., vi, vii., or ix as defined in claim 2 are determined, and wherein the at least one triacylglyceride biomarker in i., ii., and iii. is selected from the group consisting of SOP2, OSS2, SSP2, PPO1 and PPP, and/or (in particular “and”) the least one cholesterylester biomarker in i., and iii. is cholesterylester C18:2, and/or (in particular “and”) the least one phosphatidylcholine biomarker in i. and ii. is PC4, and/or (in particular “and”) the least one sphingomyelin biomarker in ii. and iii. is selected from the group consisting of SM18, SM24, SM23, SM28, SM5, and SM3.

16. The method of any one any one of the preceding claims, wherein at least the amounts of the biomarkers of panel 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, or 56 in Table 2 are determined.

17. The method of any one of the preceding claims, wherein at least the amounts of the biomarkers of i., ii., iii, vi, or ix are determined, and wherein the at least one triacylglyceride biomarker in i., ii., and iii. is SOP2 and/or OSS2, and/or (in particular “and”) wherein the at least one cholesterylester biomarker in i., and iii. is cholesterylester C18:2, and/or (in particular “and”) wherein the at least one phosphatidylcholine biomarker in i. and ii. is PC4, and/or (in particular “and”) wherein the at least one sphingomyelin biomarker in ii. and iii. is selected from the group consisting of SM18, SM24, SM23, and SM3.

18. The method of any one of the preceding claims, wherein at least the amounts of the biomarkers of panel 1, 2, 3, 4, 5, 6, 7, 8, 9, 10, 11, 12, 13, 14, 15, 16, 17 or 18 in Table 2 are determined.

19. The method of any one of the preceding claims, wherein at least the amounts of the biomarkers of i., ii., iii, vi, or ix are determined, and wherein the at least one triacylglyceride biomarker in i., ii., and iii. is SOP2 and/or OSS2, and/or (in particular “and”) wherein the at least one cholesterylester biomarker in i., and iii. is cholesterylester C18:2, and/or (in particular “and”) wherein the at least one phosphatidylcholine biomarker in i. and ii. is PC4, and/or (in particular “and”) wherein the at least one sphingomyelin biomarker in ii. and iii. is SM23.

20. The method of any one of the preceding claims, wherein at least the amounts of the biomarkers of panel 1, 2, 3 or 4 in Table 2 are determined.

21. The method of any one of the preceding claims, wherein at least the amounts of OSS2, PC4 and SM23 are determined, in particular wherein at least the amounts of OSS2, PC4 and SM23, and the amount of NT-proBNP or BNP are determined.

22. The method of any one of the preceding claims, wherein at least the amounts of OSS2, cholesterylester C18:2 and SM23 are determined.

23. The method of any one of the preceding claims, wherein at least the amounts of the biomarkers of iii. are determined, and wherein the at least one triacylglyceride biomarker is SOP2 and/or OSS2, and/or (in particular “and”) wherein the at least one cholesterylester biomarker is cholesterylester C18:2, and/or (in particular “and”) wherein the at least one sphingomyelin biomarker is SM23.

24. The method of any one of the preceding claims, wherein at least the amounts of SOP2, OSS2, PC4, Cholesterylester C18:2, SM18, SM28, SM24, SSP2, and SM23 are determined.

25. The method of any one of the preceding claims, wherein the cardiac disease is heart failure with reduced left ventricular ejection fraction is DCMP (dilated cardiomyopathy).

26. The method of claim 25, wherein at least the amounts of the lipid metabolite biomarkers shown in i., ii., iii. or vii, in particular of in i., ii., or iii. are determined, and wherein the at least one triacylglyceride biomarker in i., ii. and iii. is selected from the group consisting of SOP2, OSS2, and SSP2, and/or (in particular “and”) wherein the at least one cholesterylester biomarker in i. and iii. is cholesterylester C18:2, and/or (in particular “and”) wherein the at least one phosphatidylcholine biomarker in i. and ii. is PC4, and/or (in particular “and”) wherein the at least one sphingomyelin biomarker in ii. and iii. is selected from the group consisting of SM24, SM23, SM28, and SM3.

27. The method of claims 25 and 26, wherein at least the amounts of the biomarkers of panel 19, 20, 21, 22, 23, 24, 25, 26, 27, 28, 29, 30, 31, 32, 33, 34, 35 or 36 in Table 2 are determined.

28. The method of any one of claims 25 to 27, wherein at least the amounts of the lipid metabolite biomarkers shown in ii. or iii. are determined, and wherein the at least one triacylglyceride biomarker in ii. and iii. is SOP2 and/or OSS2, and/or (in particular “and”) wherein the at least one cholesterylester biomarker in iii. is cholesterylester C18:2, and/or (in particular “and”) wherein the at least one phosphatidylcholine biomarker in ii. is PC4, and/or (in particular “and”) wherein the at least one sphingomyelin biomarker in ii. and iii. is SM23 and/or SM24.

29. The method of any of claims 25 to 28, wherein at least the amounts of the biomarkers of panel 31, 32, 33, 34, 35 and 36 in Table 2 are determined.

30. The method of any one of the preceding claims, wherein the cardiac disease is heart failure with reduced left ventricular ejection fraction is ICMP (ischemic cardiomyopathy).

31. The method of claim 30, wherein at least the amounts of the lipid metabolite biomarkers shown in ii., iii. or vi, in particular in ii., or iii. are determined, and wherein the at least one triacylglyceride biomarker in ii. and iii. is SOP2 and/or OSS2 (in particular “and”), wherein the at least one cholesterylester biomarker in iii. is cholesterylester C18:2, and/or (in particular “and”) wherein the at least one phosphatidylcholine biomarker in ii. is PC4, and/or (in particular “and”) wherein the at least one sphingomyelin biomarker in ii. and iii. is selected from the group consisting of SM18, SM24, and SM23.

32. The method of claims 30 and 31, wherein at least the amounts of the biomarkers of panel 37, 38, 39, 40, 41, 42, 43, 44, 45, 46, 47, 48, 49, 50, 51, 52, 53, 54, 55 or 56 in Table 2 are determined.

33. The method of any one of claims 30 to 32, wherein at least the amounts of the lipid metabolite biomarkers shown in iii. are determined, and wherein the at least one triacylglyceride biomarker is SOP2, and/or (in particular “and”) wherein the at least one cholesterylester biomarker is cholesterylester C18:2, and/or (in particular “and”) wherein the at least one sphingomyelin biomarker is SM18 and/or SM23.

34. The method of any one of claims 30 to 33, wherein at least the amounts of the biomarkers of panel 51, 52, 53, 54, 55 or 56 in Table 2 are determined.

35. The method of any one of the preceding claims, wherein the cardiac disease is HfrEF and wherein the HfrEP is asymptomatic (or wherein the subject does not show symptoms of heart failure).

36. The method of claim 36, and wherein at least the amounts of the biomarkers of, ii., iii. vi, vii., or ix are determined, and wherein the at least one triacylglyceride biomarker in ii., and iii. is selected from the group consisting of SOP2, OSS2, and PPO1, and/or (in particular “and”) wherein the at least one cholesterylester biomarker in iii. is cholesterylester C18:2, and/or (in particular “and”) the least one phosphatidylcholine biomarker in ii. is PC4, and/or (in particular “and”) wherein the at least one sphingomyelin biomarker in ii. and iii. is selected from the group consisting of SM24 and SM23.

37. The method of claims 35 and 35, wherein at least the amounts of the biomarkers of panel 7, 8, 9, 10, 11, 12, 19, 20, 21, 22, 23, 24, 37, 38, 39, 40, 41 or 42 in Table 2 are determined.

38. The method of any one of claims 35 to 37, wherein at least the amounts of the biomarkers of iii. or vi. (in particular of iii.) of claim 2 are determined, and wherein the at least one triacylglyceride biomarker is SOP2, and/or (in particular “and”) wherein the at least one cholesterylester biomarker is cholesterylester C18:2, and/or (in particular “and”) wherein the at least one sphingomyelin biomarker is selected from the group consisting of SM24 and SM23.

39. The method of claim 38, wherein at least the amounts of the biomarkers of panel 7, 8, 9, 10, 11, or 12 in Table 2 are determined.

40. The method of any one of claims 1 to 10, wherein the heart failure is heart failure with preserved ejection fraction (HFpEF).

41. The method of claim 40, wherein at least the amounts of the biomarkers of ii., vi, or vii. of claim 1 are determined, and wherein the at least one triacylglyceride biomarker in ii. is selected from the group consisting of SOP2, SSP2, SPP1 and PPO1, and/or (in particular “and”) the least one phosphatidylcholine biomarker in ii. is selected from the group consisting of PC4 and PC8, and/or (in particular “and”) the least one sphingomyelin biomarker in ii. is selected from the group consisting of SM18, SM24, SM23, SM28, SM5, and SM3.

42. The method of claim 41, wherein at least the amounts of the biomarkers of panel 79, 80, 81, 82, 83, 84, 85, 86, 87, 88, 89, 90, 91, 92, 93, 94, 95, 96, 97, 98, 99, 100, 101 or 102, in Table 2 are determined.

43. The method of any one of the preceding claims, wherein the subject is a human subject.

44. The method of any one of the preceding claims, wherein the sample is blood, serum or plasma.

45. The method of any one of the preceding claims, wherein the method does not comprise the determination of NT-proBNP or BNP.

46. The method of any one of claims 1 to 44, further comprising determining the amount of NT-proBNP or BNP in a sample/the sample from the subject and comparing the amount of NT-proBNP or BNP to a reference.

47. The method of any one of claims 1 to 46, further comprising carrying out a correction for confounders.

48. The method of claim 47, wherein the confounders are age, BMI and/or gender, in particular age, BMI and gender.

49. The method of any one of the preceding claims, wherein in step b) a score is calculated based on the determined amounts of the at least three lipid metabolite biomarkers and the at least one additional cardiac biomarker, and wherein the reference is a reference score.

50. The method of any one of claims 1 to 49, wherein the reference is from a subject or group of subjects known not to suffer from cardiac disease.

51. The method of claim 50, wherein a value for each of the at least three lipid metabolite biomarkers and the at least one additional cardiac biomarker in the test sample being essentially identical as compared to the reference is indicative for the absence of cardiac disease.

52. The method of any one of claims 1 to 49, wherein the reference is from a subject or group of subjects known to suffer from heart failure.

53. The method of any one of claim 50, wherein a value for each of the at least three lipid metabolite biomarkers and the at least one additional cardiac biomarker in the test sample being essentially identical as compared to the reference is indicative for the presence of the heart failure.

54. The method of any one of claims 1 to 53, wherein the amounts of the at least three lipid metabolite biomarkers are determined by mass spectrometry (MS).

55. The method of claim 54, wherein the mass spectrometry is LC-MS, in particular LCMS/MS, or HPLC-MS, in particular HPLC-MS/MS.

56. The method of claims 54 and 55, wherein the mass spectrometry comprises an ionization step in which the at least three lipid metabolite biomarkers are ionized

57. The method of claim 56, wherein the ionization step is carried out by electrospray ionization, in particular by positive ion mode electrospray ionization.

58. A diagnostic device for carrying out the method according to any one of claims 1 to 57, comprising:
a) an analysing unit comprising at least one detector for the at least three lipid metabolite bimarkers and at least one detector for and the at least one additional cardiac biomarker in connection with the present invention detected by the at least one detector, and, operatively linked thereto;
b) an evaluation unit comprising a computer comprising tangibly embedded a computer program code for carrying out a comparison of the determined amounts of the at least three lipid metabolite biomarkers and the at least one additional cardiac biomarker with the reference amounts, and a data base comprising said reference amounts for the said biomarkers, whereby it will be diagnosed whether a subject suffers from a cardiac disease.

59. A diagnostic device for carrying out the method according to any one of claims 1 to 57, comprising:
a) an analysing unit comprising at least one detector for the at least three lipid metabolite biomarkers and at least one detector for and the at least one additional cardiac biomarker in connection with the present invention detected by the at least one detector, and, operatively linked thereto;
b) an evaluation unit comprises a computer comprising tangibly embedded a computer program code for calculating a score based on the determined amounts of the at least three lipid metabolite biomarkers and the at least one additional cardiac biomarker, and for carrying out a comparison of the calculated score and the reference score, wherein said evaluation unit further comprises a data base comprising said reference score, whereby it will be diagnosed whether a subject suffers from a cardiac disease.

60. Use of at least three lipid metabolite biomarkers and the at least one additional cardiac biomarker as set forth in the preceding claims in a sample of a subject for diagnosing cardiac disease.

61. The method of any one of claims 1 to 57, the device of claim 58 or 59 or the use of claim 60, wherein the cardiac disease is HFrEF with a left ventricular ejection fraction of lower than 50% but larger than 35%.

62. The method of any one of claim 1 to 57 or 61, the device of claim 58, 59 or 61, or the use of claim 60 or 61, wherein the subject is overweight.

63. The method of any one of claims 1 to 57 and 61 and 62, the diagnostic device of claim 58, 59, or 61 or the use of claims 60 and 61, wherein said at least three lipid metabolite biomarkers are the biomarkers of panel 1, and wherein said at least one additional cardiac biomarker is HDL cholesterol and/or LDL cholesterol, preferably wherein at least one additional cardiac biomarker is HDL cholesterol.

64. The method of claim 63, further comprising the determination of the amount of NT-proBNP in a sample/the sample from the subject and comparing the amount of NT-proBNP or BNP to a reference.
